(19) United States
(12) Patent Application Publication
(10) Pub. No.: US 2024/0110182 A1
(43) Pub. Date: Apr. 4, 2024

(54) GENOMIC EDITING OF COMPLEMENT

(71) Applicant: Apellis Pharmaceuticals, Inc., Waltham, MA (US)

(72) Inventors: Tara Barbour, Zug (CH); Pascal Deschatelets, West Newton, MA (US); Lukas Scheibler, Telluride, CO (US)

(73) Assignee: Apellis Pharmaceuticals, Inc., Waltham, MA (US)

(21) Appl. No.: 18/266,464

(22) PCT Filed: Dec. 10, 2021
(86) PCT No.: PCT/US21/62896

§ 371 Date: Jun. 9, 2023

Related U.S. Application Data
(60) Provisional application No. 63/124,009, filed on Dec. 10, 2020.

(51) Int. Cl. C12N 15/113; A61K 48/00; C12N 9/22; C12N 9/78; C12N 15/90

(52) U.S. Cl. CPC C12N 15/113; A61K 48/005; C12N 9/22; C12N 9/78; C12N 15/907; C07K 2319/00; C12N 2310/20

(57) ABSTRACT

Methods, systems, and compositions for genomic editing of a gene encoding a complement protein, e.g., C3, are disclosed.

### CROSS REFERENCE TO RELATED APPLICATIONS

This application claims the benefit of U.S. Provisional Application No. 63/124,009, filed Dec. 10, 2020, the contents of which are herein incorporated by reference in their entirety.

### BACKGROUND

Complement is a system consisting of more than 30 plasma and cell-bound proteins that plays a significant role in both innate and adaptive immunity. The proteins of the complement system act in a series of enzymatic cascades through a variety of protein interactions and cleavage events. Complement activation occurs via three main pathways: the antibody-dependent classical pathway, the alternative pathway, and the mannose-binding lectin (MBL) pathway. Inappropriate or excessive complement activation is an underlying cause or contributing factor to a number of serious diseases and conditions, and considerable effort has been devoted over the past several decades to exploring various complement inhibitors as therapeutic agents.

### SUMMARY

In one aspect, the disclosure features a method of treating a subject, comprising administering to a cell of a subject: (i) a base editor comprising a fusion protein comprising an endonuclease (e.g., a Cas endonuclease) and a deaminase; and (ii) a gRNA (e.g., a single guide RNA (sgRNA)) comprising a targeting domain comprising a nucleotide sequence that is complementary to a portion of a human C3 gene, wherein after the administering step, the cell and/or the subject exhibits reduced expression and/or activity of C3 protein (e.g., reduced by about 10%, 20%, 30%, 40%, 50%, 60%, 70%, 80%, or 90%), relative to a control.

In some embodiments, the portion of the human C3 gene comprises a nucleotide sequence within an exon of SEQ ID NO:1. In some embodiments, the portion of the human C3 gene comprises a nucleotide sequence within an intron of SEQ ID NO:1.

In some embodiments, the gRNA targets the base editor to one or more base positions recited in Table 2, 3 or 4. In some embodiments, after the administering step, the human C3 gene comprises a base edit, relative to a wildtype human C3 gene, from a C to a T; from a G to an A; from a T to a C; or from an A to a G at one or more base positions recited in Table 2, 3 or 4. In some embodiments, after the administering step, the human C3 gene comprises a genomic edit, relative to a wildtype human C3 gene, of a nonstop codon to a stop codon at one or more base positions recited in Table 2, 3, or 4.

In some embodiments, the reduced activity of the C3 protein comprises reduced thioester domain activity.

In some embodiments, after the administering step, the cell or the subject expresses a mutant C3 protein, and a level or rate of cleavage of the mutant C3 protein by a C3 convertase is reduced (e.g., reduced by about 10%, 20%, 30%, 40%, 50%, 60%, 70%, 80%, or 90%), relative to level or rate of cleavage of a wildtype C3 protein by the C3 convertase.

In some embodiments, the Cas endonuclease is a nuclease inactive Cas endonuclease. In some embodiments, the Cas endonuclease is a nickase. In some embodiments, the nickase is a Cas9 nickase.

In some embodiments, the deaminase is a deaminase from the apolipoprotein B mRNA-editing complex (APOBEC) family deaminase. In some embodiments, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase.

In some embodiments, the method comprises administering a nucleotide sequence encoding the base editor. In some embodiments, the method comprises administering a viral vector comprising the nucleotide sequence encoding the base editor.

In some embodiments, the method comprises administering a viral vector comprising the gRNA.

In some embodiments, the method comprises administering a viral vector comprising the nucleotide sequence encoding the base editor and comprising the gRNA.

In some embodiments, the method comprises administering a ribonucleoprotein (RNP) complex comprising the base editor and the gRNA.

In some embodiments, the subject has or suffers from or is at risk of developing a complement-mediated disorder.

In another aspect, the disclosure features a method of editing a human C3 gene in a cell, comprising contacting a cell with, or administering to a subject: (i) a base editor comprising a fusion protein comprising an endonuclease (e.g., a Cas endonuclease) and a deaminase; and (ii) a gRNA (e.g., a single guide RNA (sgRNA)) comprising a targeting domain comprising a nucleotide sequence that is complementary to a portion of the human C3 gene, wherein after the contacting or administering step, the cell comprises a human C3 gene comprising at least one genomic edit.

In some embodiments, after the administering step, the cell and/or the subject exhibits reduced expression and/or activity of C3 protein (e.g., reduced by about 10%, 20%, 30%, 40%, 50%, 60%, 70%, 80%, or 90%), relative to a control.

In some embodiments, the portion of the human C3 gene comprises a nucleotide sequence within an exon of SEQ ID NO:1. In some embodiments, the portion of the human C3 gene comprises a nucleotide sequence within an intron of SEQ ID NO:1.

In some embodiments, the gRNA targets the base editor to one or more base positions recited in Table 2, 3 or 4. In some embodiments, after the administering step, the human C3 gene comprises a base edit, relative to a wildtype human C3 gene, from a C to a T; from a G to an A; from a T to a C; or from an A to a G at one or more base positions recited in Table 2, 3 or 4. In some embodiments, after the administering step, the human C3 gene comprises a genomic edit, relative to a wildtype human C3 gene, of a nonstop codon to a stop codon at one or more base positions recited in Table 2, 3, or 4.

In some embodiments, the reduced activity of the C3 protein comprises reduced thioester domain activity. In some embodiments, after the administering step, the cell or the subject expresses a mutant C3 protein, and a level or rate of cleavage of the mutant C3 protein by a C3 convertase is reduced (e.g., reduced by about 10%, 20%, 30%, 40%, 50%, 60%, 70%, 80%, or 90%), relative to level or rate of cleavage of a wildtype C3 protein by the C3 convertase.

In some embodiments, the Cas endonuclease is a nuclease inactive Cas endonuclease. In some embodiments, the Cas endonuclease is a nickase. In some embodiments, the nickase is a Cas9 nickase.

In some embodiments, the deaminase is a deaminase from the apolipoprotein B mRNA-editing complex (APOBEC) family deaminase. In some embodiments, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase.

In some embodiments, the method comprises administering a nucleotide sequence encoding the base editor. In some embodiments, the method comprises administering a viral vector comprising the nucleotide sequence encoding the base editor.

In some embodiments, the method comprises administering a viral vector comprising the gRNA.

In some embodiments, the method comprises administering a viral vector comprising the nucleotide sequence encoding the base editor and comprising the gRNA.

In some embodiments, the method comprises administering a ribonucleoprotein (RNP) complex comprising the base editor and the gRNA.

In some embodiments, the subject has or suffers from or is at risk of developing a complement-mediated disorder.

In another aspect, the disclosure features a composition comprising: (i) a base editor comprising a fusion protein comprising an endonuclease (e.g., a Cas endonuclease) and a deaminase; and (ii) a gRNA (e.g., a single guide RNA (sgRNA)) comprising a targeting domain comprising a nucleotide sequence that is complementary to a portion of the human C3 gene. In some embodiments, the gRNA targets the base editor to one or more base positions recited in Table 2, 3 or 4.

In another aspect, the disclosure features a cell comprising a composition comprising: (i) a base editor comprising a fusion protein comprising an endonuclease (e.g., a Cas endonuclease) and a deaminase; and (ii) a gRNA (e.g., a single guide RNA (sgRNA)) comprising a targeting domain comprising a nucleotide sequence that is complementary to a portion of the human C3 gene; or a progeny of such cell. In some embodiments, the gRNA targets the base editor to one or more base positions recited in Table 2, 3 or 4.

In another aspect, the disclosure features a method of reducing complement activation in a subject (e.g., reducing by about 10%, 20%, 30%, 40%, 50%, 60%, 70%, 80%, or 90%), relative to a control, the method comprising administering to the subject a composition comprising: (i) a base editor comprising a fusion protein comprising an endonuclease (e.g., a Cas endonuclease) and a deaminase; and (ii) a gRNA (e.g., a single guide RNA (sgRNA)) comprising a targeting domain comprising a nucleotide sequence that is complementary to a portion of the human C3 gene. In some embodiments, the gRNA targets the base editor to one or more base positions recited in Table 2, 3 or 4.

In some embodiments of any of the aspects described herein, the cell is a hepatic cell.

### Definitions

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Subject: As used herein, the term “subject” or “test subject” refers to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Suffering from: An individual who is “suffering from” a disease, disorder, and/or condition has been diagnosed with and/or displays one or more symptoms of a disease, disorder, and/or condition.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a complement-mediated disorder or is at increased risk of developing such a disorder relative to a member of the general population. A composition of the disclosure can be administered prophylactically, i.e., before development of any symptom or manifestation of the condition. Typically in this case the subject will be at risk of developing the condition.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, the prefix poly—refers to a nucleic acid containing 2 to about 10,000, 2 to about 50,000, or 2 to about 100,000 nucleotide monomer units. In some embodiments, the prefix oligo—refers to a nucleic acid containing 2 to about 200 nucleotide monomer units.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Endogenous: The term “endogenous,” as used herein in the context of nucleic acids (e.g., genes, protein-encoding genomic regions, promoters), refers to a native nucleic acid or protein in its natural location, e.g., within the genome of a cell.

Exogenous: The term “exogenous,” as used herein in the context of nucleic acids, e.g., expression constructs, cDNAs, indels, and nucleic acid vectors, refers to nucleic acids that have artificially been introduced into the genome of a cell using, for example, gene-editing or genetic engineering techniques, e.g., CRISPR-based editing techniques.

Guide RNA: The terms “guide RNA” and “gRNA” refer to any nucleic acid that promotes the specific association (or “targeting”) of an endonuclease such as a Cas9 or a Cpf1 to a target sequence such as a genomic or episomal sequence in a cell.

Mutant: The term “mutant” or “variant” as used herein refers to an entity such as a polypeptide, polynucleotide or small molecule that shows significant structural identity with a reference entity but differs structurally from the reference entity in the presence or level of one or more chemical moieties as compared with the reference entity. In many embodiments, a mutant or variant also differs functionally from its reference entity. In general, whether a particular entity is properly considered to be a “variant” of a reference entity is based on its degree of structural identity with the reference entity.

Conventional IUPAC notation is used in nucleotide sequences presented herein, as shown in Table 10, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 10
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### BRIEF DESCRIPTION OF THE DRAWING

FIG. 1 shows the structure of pegcetacoplan (“APL-2”), assuming n of about 800 to about 1100 and a PEG of about 40 kD.

### DETAILED DESCRIPTION OF CERTAIN EMBODIMENTS

The present disclosure encompasses, in part, methods, systems, and compositions for genetically engineering, e.g., by genomic editing, one or more genes encoding a complement protein described herein. Such methods can be used, e.g., to treat a subject having or at risk of a complement-mediated disorder.

### Complement System

Complement is a system consisting of numerous plasma and cell-bound proteins that plays a significant role in both innate and adaptive immunity. The proteins of the complement system act in a series of enzymatic cascades through a variety of protein interactions and cleavage events. To facilitate understanding of the disclosure, and without intending to limit the invention in any way, this section provides an overview of complement and its pathways of activation. Further details are found, e.g., in Kuby Immunology, 6th ed., 2006; Paul, W. E., Fundamental Immunology, Lippincott Williams & Wilkins; 6th ed., 2008; and Walport M J., Complement. First of two parts. N Engl J Med., 344(14):1058-66, 2001.

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 at a defined cleavage site to form C3a and C3b (see, e.g., Kulkarni et al., Am J Respir Cell Mol Biol 60:144-157 (2019)). Binding of C3b to C3 convertase produces C5 convertase, which cleaves C5 into C5a and C5b. C3a, C4a, and C5a are anaphylatoxins and mediate multiple reactions in the acute inflammatory response. C3a and C5a are also chemotactic factors that attract immune system cells such as neutrophils. It will be understood that the names “C2a” and “C2b” used initially were subsequently reversed in the scientific literature.

The alternative pathway is initiated by and amplified at, e.g., microbial surfaces and various complex polysaccharides. In this pathway, hydrolysis of C3 to C3 (H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC), also known as the terminal complement complex (TCC). The MAC inserts itself into target cell membranes and causes cell lysis. Small amounts of MAC on the membrane of cells may have a variety of consequences other than cell death. If the TCC does not insert into a membrane, it can circulate in the blood as soluble sC5b-9 (sC5b-9). Levels of sC5b-9 in the blood may serve as an indicator of complement activation.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Inappropriate or excessive complement activation is an underlying cause or contributing factor to a number of serious diseases and conditions. Deficiencies or defects in complement regulatory protein(s) are involved in the pathogenesis of a variety of complement-mediated disorders.

### Genome Editing Systems and Techniques

In some embodiments, genetic engineering is performed on a cell, e.g., of a subject in need of a reduction of level of expression or activity of complement (e.g., a subject suffering from or at risk of a complement mediated disorder). In some embodiments, genetic engineering is performed using genome editing.

As used herein, “genome editing” refers to a method of modifying a genome, including any protein-coding or non-coding nucleotide sequence, of an organism to modify and/or knock out expression of a target gene. In general, genome editing methods involve use of an endonuclease that is capable of cleaving the nucleic acid of a genome, for example at a targeted nucleotide sequence. Repair of single- or double-stranded breaks in the genome may introduce mutations and/or exogenous nucleic acid may be inserted into the targeted site.

Genome editing methods are known in the art and are generally classified based on type of endonuclease that is involved in generating breaks in a target nucleic acid. These methods include, e.g., use of zinc finger nucleases (ZFN), transcription activator-like effector-based nuclease (TALEN), meganucleases, and CRISPR/Cas systems.

In some embodiments, genome editing methods utilize TALEN technology known in the art. In general, TALENs are engineered restriction enzymes that can specifically bind and cleave a desired target DNA molecule. A TALEN typically contains a Transcriptional Activator-Like Effector (TALE) DNA-binding domain fused to a DNA cleavage domain. The DNA binding domain may contain a highly conserved 33-34 amino acid sequence with a divergent 2 amino acid RVD (repeat variable dipeptide motif) at positions 12 and 13. The RVD motif determines binding specificity to a nucleic acid sequence and can be engineered according to methods known to those of skill in the art to specifically bind a desired DNA sequence. In one example, the DNA cleavage domain may be derived from the FokI endonuclease. The FokI domain functions as a dimer, requiring two constructs with unique DNA binding domains for sites in the target genome with proper orientation and spacing. TALENs specific to sequences in a target gene of interest (e.g., C3) can be constructed using any method known in the art.

A TALEN specific to a target gene of interest can be used inside a cell to produce a double-stranded break (DSB). A mutation can be introduced at the break site if the repair mechanisms improperly repair the break via non-homologous end joining. For example, improper repair may introduce a frame shift mutation. Alternatively, a foreign DNA molecule having a desired sequence can be introduced into the cell along with the TALEN. Depending on the sequence of the foreign DNA and chromosomal sequence, this process can be used to correct a defect or introduce a DNA fragment into a target gene of interest, or introduce such a defect into an endogenous gene, thus decreasing expression of the target gene.

In some embodiments, cells can be genetically manipulated using zinc finger (ZFN) technology known in the art. In general, zinc finger mediated genomic editing involves use of a zinc finger nuclease, which typically comprises a DNA binding domain (i.e., zinc finger) and a cleavage domain (i.e., nuclease). The zinc finger binding domain may be engineered to recognize and bind to any target gene of interest (e.g., C3) using methods known in the art and in particular, may be designed to recognize a DNA sequence ranging from about 3 nucleotides to about 21 nucleotides in length, or from about 8 to about 19 nucleotides in length. Zinc finger binding domains typically comprise at least three zinc finger recognition regions (e.g., zinc fingers). Restriction endonucleases (restriction enzymes) capable of sequence-specific binding to DNA (at a recognition site) and cleaving DNA at or near the site of binding are known in the art and may be used to form ZFN for use in genomic editing. For example, Type IIS restriction endonucleases cleave DNA at sites removed from the recognition site and have separable binding and cleavage domains. In some embodiments, the DNA cleavage domain may be derived from FokI endonuclease.

In some embodiments, genomic editing is performed using a CRISPR-Cas system, where the Clustered Regularly Interspaced Short Palindromic Repeats (CRISPR)-Cas system is an engineered, non-naturally occurring CRISPR-Cas system. A CRISPR-Cas system can hybridize with a target sequence in a polynucleotide encoding a complement protein described herein, e.g., C3, allowing the cleavage of and modifying the polynucleotide. CRISPR/Cas system comprises a Cas endonuclease and an engineered crRNA/tracrRNA (or single guide RNA). In some embodiments, the CRISPR/Cas system includes a crRNA and does not include a tracrRNA sequence.

A CRISPR/Cas system of the present disclosure may bind to and/or cleave a region of interest within a coding or non-coding region, within or adjacent to a gene, such as, for example, a leader sequence, trailer sequence or intron, or within a non-transcribed region, either upstream or downstream of a coding region. The guide RNAs (gRNAs) used in the present disclosure may be designed such that the gRNA directs binding of the Cas enzyme-gRNA complexes to a pre-determined cleavage sites (target site) in a genome. The cleavage sites may be chosen so as to release a fragment that contains a region of unknown sequence, or a region containing a SNP, nucleotide insertion, nucleotide deletion, rearrangement, etc.

Cleavage of a gene region may comprise cleaving one or two strands at the location of the target sequence by the Cas enzyme. In some embodiments, such cleavage can result in decreased transcription of a target gene. In some embodiments, cleavage can further comprise repairing the cleaved target polynucleotide by homologous recombination with an exogenous template polynucleotide, wherein the repair results in an insertion, deletion, or substitution of one or more nucleotides of the target polynucleotide.

The terms “gRNA”, “guide RNA” and “CRISPR guide sequence” are used interchangeably herein and refer to a nucleic acid comprising a sequence that determines the specificity of a Cas DNA binding protein of a CRISPR/Cas system. A gRNA hybridizes to (complementary to, partially or completely) a target nucleic acid sequence in a genome of a host cell. Methods of designing and constructing gRNAs are known in the art, which can be modified to produce gRNAs that bind to a target sequence described herein (see, e.g., U.S. Pat. No. 8,697,359). The gRNA or portion thereof that hybridizes to the target nucleic acid may be about 15 to about 25 nucleotides, about 18 to about 22 nucleotides, or about 19 to about 21 nucleotides in length. In some embodiments, a gRNA sequence that hybridizes to a target nucleic acid is about 15, 16, 17, 18, 19, 20, 21, 22, 23, 24, or 25 nucleotides in length. In some embodiments, a gRNA sequence that hybridizes to a target nucleic acid is about 10 to about 30, or about 15 to about 25, nucleotides in length.

In addition to a sequence that binds to a target nucleic acid, in some embodiments, a gRNA also comprises a scaffold sequence. Expression of a gRNA encoding both a sequence complementary to a target nucleic acid and scaffold sequence has a dual function of both binding (hybridizing) to a target nucleic acid and recruiting an endonuclease to the target nucleic acid, which may result in site-specific CRISPR activity. In some embodiments, such a chimeric gRNA is referred to as a single guide RNA (sgRNA).

As used herein, a “scaffold sequence”, also referred to as a tracrRNA, refers to a nucleic acid sequence that recruits a Cas endonuclease to a target nucleic acid bound (hybridized) to a complementary gRNA sequence. Any scaffold sequence that comprises at least one stem loop structure and recruits an endonuclease may be used in the genetic elements and vectors described herein. Exemplary scaffold sequences are known in the art and described in, for example, Jinek et al., Science (2012) 337(6096):816-821, Ran et al., Nature Protocols (2013) 8:2281-2308, PCT Publication No. WO2014/093694, and PCT Publication No. WO2013/176772. In some embodiments, the CRISPR-Cas system does not include a tracrRNA sequence.

In some embodiments, a gRNA sequence does not comprise a scaffold sequence, and a scaffold sequence is expressed as a separate transcript. In some embodiments, a gRNA sequence further comprises an additional sequence that is complementary to a portion of a scaffold sequence and functions to bind (hybridize) a scaffold sequence and recruit a endonuclease to a target nucleic acid.

In some embodiments, a gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, a gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′end of the target nucleic acid). As will be evident to one of ordinary skill in the art, selection of sgRNA sequences may depend on factors such as the number of predicted on-target and/or off-target binding sites. In some embodiments, the sgRNA sequence is selected to maximize potential on-target and minimize potential off-target sites. As would be evident to one of ordinary skill in the art, various tools may be used to design and/or optimize the sequence of a sgRNA, for example to increase the specificity and/or precision of genomic editing. In general, candidate sgRNAs may be designed by identifying a sequence within the target region that has a high predicted on-target efficiency and low off-target efficiency based on any of the available web-based tools. Candidate sgRNAs may be further assessed by manual inspection and/or experimental screening. Examples of web-based tools include, without limitation, CRISPR seek, CRISPR Design Tool, Cas-OFFinder, E-CRISP, ChopChop, CasOT, CRISPR direct, CRISPOR, BREAKING-CAS, CrispRGold, and CCTop. See, e.g., Safari, et al. Current Pharma. Biotechol. (2017) 18(13).

In some embodiments, the Cas endonuclease is a Cas9 nuclease (or variant thereof) or a Cpf1 nuclease (or variant thereof). Cas9 endonucleases cleave double stranded DNA of a target nucleic acid resulting in blunt ends, whereas cleavage with Cpf1 nucleases results in staggered ends of the nucleic acid. Cas9 nuclease sequences and structures are known to those of skill in the art (see, e.g., Ferretti et al., PNAS 98:4658-4663 (2001); Deltcheva et al., Nature 471:602-607 (2011); Jinek et al., Science 337:816-821 (2012). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., (2013) RNA Biology 10:5, 726-737. In some embodiments, wild type Cas9 corresponds to Cas9 from Streptococcus pyogenes (NCBI Reference Sequence: NC_002737.2, nucleotide); and Uniprot Reference Sequence: Q99ZW2 (amino acid). In some embodiments, wild type Cas9 corresponds to Cas9 from Staphylococcus aureus (NCBI Reference Sequence: WP_001573634.1, amino acid). In some embodiments, Cas9 refers to Cas9 from: Corynebacteriumulcerans (NCBI Refs: NC_015683.1, NC_017317.1); Corynebacteriumdiphtheria (NCBI Refs: NC_016782.1, NC_016786.1); Spiroplasmasyrphidicola (NCBI Ref: NC_021284.1); Prevotella intermedia (NCBI Ref:NC_017861.1); Spiroplasma taiwanense (NCBI Ref: NC_021846.1); Streptococcus iniae (NCBI Ref: NC_021314.1); Belliella baltica (NCBI Ref:NC_018010.1); Psychroflexus torquisl (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria, meningitidis (NCBI Ref: YP_002342100.1).

A target nucleic acid may be flanked on the 3′ side by a protospacer adjacent motif (PAM), which may interact with an endonuclease and may be involved in targeting endonuclease activity to the target nucleic acid. It is generally thought that a PAM sequence flanking a target nucleic acid depends on the endonuclease and the source from which the endonuclease is derived. For example, for Cas9 endonucleases that are derived from Streptococcus pyogenes, the PAM sequence is NGG. For Cas9 endonucleases derived from Staphylococcus aureus, the PAM sequence is NNGRRT. For Cas9 endonucleases that are derived from Neisseria meningitidis, the PAM sequence is NNNNGATT. For Cas9 endonucleases derived from Streptococcus thermophilus, the PAM sequence is NNAGAA. For Cas9 endonuclease derived from Treponema denticola, the PAM sequence is NAAAAC. For a Cpf1 nuclease, the PAM sequence is TTTN. In some embodiments, the Cas endonuclease is MAD7 (also referred to as Cpf1 nuclease from Eubacterium rectale) and the PAM sequence is YTTTN.

In some embodiments, a Cas endonuclease is a Cas9 enzyme or variant thereof. In some embodiments, a Cas9 endonuclease is derived from Streptococcus pyogenes, Staphylococcus aureus, Neisseria meningitidis, Streptococcus thermophilus, Campylobacter jujuni or Treponema denticola. In some embodiments, a nucleotide sequence encoding the Cas endonuclease is codon optimized for expression in a host cell. In some embodiments, an endonuclease is a Cas9 homolog or ortholog.

In some embodiments, wild-type or mutant Cas enzyme may be used. In some embodiments, a nucleotide sequence encoding a Cas9 enzyme is modified to alter activity of the protein. A mutant Cas enzyme may lack the ability to cleave one or both strands of a target polynucleotide containing a target sequence. Cas9 harbors two independent nuclease domains homologous to HNH and RuvC endonucleases, and by mutating either of the two domains, the Cas9 protein can be converted to a nickase that introduces single-strand breaks (Cong, L. et al. Science 339, 819-823 (2013)). For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, D10A, H840A, N854A, N863A, and combinations thereof. “nCas9”, which is a point mutant (D10A) of wild-type Cas9 nuclease, has nickase activity. “dCas9”, which contains mutations D10A and H840A, lacks endonuclease activity. See, e.g., Dabrowska et al. Frontiers in Neuroscience (2018) 12(75). In some embodiments, the Cas9 nickase comprises a mutation at amino acid position D10 and/or H840. In some embodiments, the Cas9 nickase comprises the substitution mutation D10A and/or H840A.

In some embodiments, a Cas9 endonuclease is a catalytically inactive Cas9 (e.g., dCas9). Alternatively or in addition, a Cas9 endonuclease may be fused to another protein or portion thereof. In some embodiments, dCas9 is fused to a repressor domain, such as a KRAB domain. In some embodiments, dCas9 is fused to an activator domain, such as VP64 or VPR. In some embodiments, dCas9 is fused to an epigenetic modulating domain, such as a histone demethylase domain or a histone acetyltransferase domain. In some embodiments, dCas9 is fused to a LSD1 or p300, or a portion thereof. In some embodiments, dCas9 or Cas9 is fused to a Fok1 nuclease domain. In some embodiments, Cas9 or dCas9 is fused to a fluorescent protein (e.g., GFP, vRFP, mCherry, etc.).

In some embodiments, the Cas endonuclease is modified to enhance specificity of the enzyme (e.g., reduce off-target effects, maintain robust on-target cleavage). In some embodiments, the Cas endonuclease is an enhanced specificity Cas9 variant (e.g., eSPCas9). See, e.g., Slaymaker et al. Science (2016) 351 (6268): 84-88. In some embodiments, the Cas endonuclease is a high fidelity Cas9 variant (e.g., SpCas9-HF1). See, e.g., Kleinstiver et al. Nature (2016) 529: 490-495.

In some embodiments, a nucleotide sequence encoding the Cas endonuclease is modified further to alter the specificity of the endonuclease activity (e.g., reduce off-target cleavage, decrease the Cas endonuclease activity or lifetime in cells, increase homology-directed recombination and/or reduce non-homologous end joining). See, e.g., Komor et al. Cell (2017) 168: 20-36. In some embodiments, the nucleotide sequence encoding the Cas endonuclease is modified to alter the PAM recognition of the endonuclease. For example, the Cas endonuclease SpCas9 recognizes PAM sequence NGG, whereas relaxed variants of the SpCas9 comprising one or more modifications of the endonuclease (e.g., VQR SpCas9, EQR SpCas9, VRER SpCas9) may recognize the PAM sequences NGA, NGAG, NGCG. PAM recognition of a modified Cas endonuclease is considered “relaxed” if the Cas endonuclease recognizes more potential PAM sequences as compared to the Cas endonuclease that has not been modified. For example, the Cas endonuclease SaCas9 recognizes PAM sequence NNGRRT, whereas a relaxed variant of the SaCas9 comprising one or more modifications of the endonuclease (e.g., KKH SaCas9) may recognize the PAM sequence NNNRRT. In one example, the Cas endonuclease FnCas9 recognizes PAM sequence NNG, whereas a relaxed variant of the FnCas9 comprising one or more modifications of the endonuclease (e.g., RHA FnCas9) may recognize the PAM sequence YG. In one example, the Cas endonuclease is a Cpflendonuclease comprising substitution mutations S542R and K607R and recognize the PAM sequence TYCV. In one example, the Cas endonuclease is a Cpf1 endonuclease comprising substitution mutations S542R, K607R, and N552R and recognize the PAM sequence TATV. See, e.g., Gao et al. Nat. Biotechnol. (2017) 35(8): 789-792.

In some embodiments, a Cas endonuclease is a Cpf1 nuclease. In some embodiments, a Cpf1 nuclease is derived from Prevotella spp. or Francisella spp. In some embodiments, the nucleotide sequence encoding a Cpf1 nuclease is codon optimized for expression in a host cell.

In some embodiments, an endonuclease is a base editor. As described herein, the term “base editor” refers to a protein that edits a nucleotide base. “Base edit” refers to the conversion of one nucleobase to another (e.g., A to G, A to C, A to T, C to T, C to G, C to A, G to A, G to C, G to T, T to A, T to C, T to G). A base editor endonuclease generally comprises a catalytically inactive Cas endonuclease, or a Cas endonuclease with reduced catalytic activity, fused to a function domain. See, e.g., Eid et al., Biochem. J. (2018) 475(11): 1955-1964; Rees et al. Nature Reviews Genetics (2018)19:770-788. In some embodiments, the catalytically inactive Cas endonuclease is dCas9. In some embodiments, the endonuclease comprises a dCas9 fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytodine deaminase enzyme (e.g., APOBEC deaminase, pmCDA1, activation-induced cytidine deaminase (AID)). In some embodiments, the Cas endonuclease has reduced activity and is nCas9. In some embodiments, the endonuclease comprises a nCas9 fused to one or more uracil glycosylase inhibitor (UGI) domains. In some embodiments, the endonuclease comprises a nCas9 fused to an adenine base editor (ABE), for example an ABE evolved from the RNA adenine deaminase TadA. In some embodiments, the endonuclease comprises a nCas9 fused to cytodine deaminase enzyme (e.g., APOBEC deaminase, pmCDA1, activation-induced cytidine deaminase (AID)). In some embodiments, a base editor comprises a fusion protein comprising (i) a Cas9 (e.g., dCas9 or nCas9), CasX, CasY, Cpf1, C2c1, C2c2, C2c3, or Argonaute protein; (ii) a deaminase (e.g., a deaminase from the apolipoprotein B mRNA-editing complex (APOBEC) family deaminase, e.g., APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, or APOBEC3H deaminase); and (iii) a UGI domain. In some embodiments, a base editor described herein further comprises a nuclear localization signal.

Examples of base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in US 20170121693, US 20180312825, US 20180312828, PCT Publication No. WO 2018165629A1, and Porto et al., Nat Rev Drug Discov. 19:839-859 (2020).

A catalytically inactive variant of Cpf1 (Cas12a) may be referred to dCas12a. As described herein, catalytically inactive variants of Cpf1 may be fused to a function domain to form a base editor. See, e.g., Rees et al. Nature Reviews Genetics (2018) 19:770-788. In some embodiments, the catalytically inactive Cas endonuclease is dCas9. In some embodiments, the endonuclease comprises a dCas12a fused to one or more uracil glycosylase inhibitor (UGI) domains. In some embodiments, the endonuclease comprises a dCas12a fused to an adenine base editor (ABE), for example an ABE evolved from the RNA adenine deaminase TadA. In some embodiments, the endonuclease comprises a dCas12a fused to cytodine deaminase enzyme (e.g. APOBEC deaminase, pmCDA1, activation-induced cytidine deaminase (AID)). Alternatively or in addition, the Cas endonuclease may be a Cas14 endonuclease or variant thereof. In contrast to Cas9 endonucleases, Cas14 endonucleases are derived from archaea and tend to be smaller in size (e.g., 400-700 amino acids). Additionally Cas14 endonucleases do not require a PAM sequence. See, e.g., Harrington et al., Science 362:839-842 (2018).

Also provided herein are methods of producing genetically engineered cells described herein, which carry one or more edited genes encoding one or more complement protein (e.g., C3). In some embodiments, methods include providing a cell and introducing into the cell components of a CRISPR Cas system for genome editing. In some embodiments, a nucleic acid that comprises a CRISPR-Cas guide RNA (gRNA) that hybridizes or is predicted to hybridize to a portion of the nucleotide sequence that encodes a complement protein (e.g., C3) is introduced into the cell. In some embodiments, the gRNA is introduced into the cell via a vector. In some embodiments, a Cas endonuclease is introduced into the cell. In some embodiments, the Cas endonuclease is introduced into the cell as a nucleic acid encoding a Cas endonuclease. In some embodiments, the gRNA and a nucleotide sequence encoding a Cas endonuclease are introduced into the cell within a single nucleic acid (e.g., the same vector). In some embodiments, the gRNA and a nucleotide sequence encoding a Cas endonuclease are introduced into the cell within separate nucleic acids (e.g., different vectors). In some embodiments, the Cas endonuclease is introduced into the cell in the form of a protein. In some embodiments, the Cas endonuclease and the gRNA are pre-formed in vitro and are introduced to the cell in as a ribonucleoprotein complex.

In some embodiments, multiple gRNAs are introduced into the cell. In some embodiments, the two or more guide RNAs are transfected into cells in equimolar amounts. In some embodiments, the two or more guide RNAs are provided in amounts that are not equimolar. In some embodiments, the two or more guide RNAs are provided in amounts that are optimized so that editing of each target occurs at equal frequency. In some embodiments, the two or more guide RNAs are provided in amounts that are optimized so that editing of each target occurs at optimal frequency.

Vectors of the present disclosure can drive the expression of one or more sequences in mammalian cells using a mammalian expression vector. Examples of mammalian expression vectors include pCDM8 (Seed, Nature (1987) 329: 840) and pMT2PC (Kaufman, et al., EMBO J. (1987) 6: 187). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL 2nd eds., Cold Spring Harbor Laboratory, Cold Spring Harbor Laboratory Press, Cold Spring Harbor, N.Y., 1989.

In some embodiments, vectors described herein are capable of directing expression of nucleic acids preferentially in a particular cell type (e.g., tissue-specific regulatory elements are used to express the nucleic acid). Such regulatory elements include promoters that may be tissue specific or cell specific. Specificity of a promoter may be assessed using methods well known in the art, e.g., immunohistochemical staining.

Conventional viral and non-viral based gene transfer methods can be used to introduce nucleic acids encoding an endonuclease described herein (e.g., ZFN, TALEN, meganucleases, and CRISPR-Cas9) in mammalian cells or target tissues. For example, such methods can be used to administer nucleic acids encoding components of a CRISPR-Cas system to cells in culture, or in a host organism. Non-viral vector delivery systems include DNA plasmids, RNA (e.g., a transcript of a vector described herein), naked nucleic acid, and nucleic acid complexed with a delivery vehicle. In some embodiments, nucleic acids encoding CRISPR/Cas9 are introduced by transfection (e.g., electroporation, microinjection). In some embodiments, nucleic acids encoding CRISPR/Cas9 are introduced by nanoparticle delivery, e.g., cationic nanocarriers. In some embodiments, nucleic acids encoding CRISPR/Cas9 are introduced by lipid nanoparticles.

Viral vector delivery systems include DNA and RNA viruses, which have either episomal or integrated genomes after delivery to the cell.

Viral vectors can be administered directly to subjects (in vivo) or they can be used to manipulate cells in vitro or ex vivo, where the modified cells may be administered to patients. Viral vectors include, but are not limited to, retroviral, lentivirus, adenoviral, adeno-associated and herpes simplex virus vectors for gene transfer. Furthermore, the present disclosure provides vectors capable of integration in the host genome, such as retrovirus or lentivirus. In vivo, many complement proteins, including C3, are synthesized primarily in the liver. As such, in some embodiments, hepatocytes are targeted for genomic editing. Several classes of viral vectors have been shown competent for liver-targeted delivery of a gene therapy construct, including retroviral vectors (see, e.g., Axelrod et al., PNAS 87:5173-5177 (1990); Kay et al., Hum. Gene Ther. 3:641-647 (1992); Van den Driessche et al., PNAS 96:10379-10384 (1999); Xu et al., ASAIO J. 49:407-416 (2003); and Xu et al., PNAS 102:6080-6085 (2005)), lentiviral vectors (see, e.g., McKay et al., Curr. Pharm. Des. 17:2528-2541 (2011); Brown et al., Blood 109:2797-2805 (2007); and Matrai et al., Hepatology 53:1696-1707 (2011)), adeno-associated viral (AAV) vectors (see, e.g., Herzog et al., Blood 91:4600-4607 (1998)), and adenoviral vectors (see, e.g., Brown et al., Blood 103:804-810 (2004) and Ehrhardt et al., Blood 99:3923-3930 (2002)).

In some embodiments, regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken R-actin promoter, a pol II promoter, or a pol III promoter.

In some embodiments, a viral vector is designed for genomic editing of hepatocytes, and a viral vector includes one or more liver-specific regulatory elements, which substantially limit expression to hepatic cells. Generally, liver-specific regulatory elements can be derived from any gene known to be exclusively expressed in the liver. WO 2009/130208 identifies several genes expressed in a liver-specific fashion, including serpin peptidase inhibitor, clade A member 1, also known as α-antitrypsin (SERPINA1; GeneID 5265), apolipoprotein C-I (APOC1; GeneID 341), apolipoprotein C-IV (APOC4; GeneID 346), apolipoprotein H (APOH; GeneID 350), transthyretin (TTR; GeneID 7276), albumin (ALB; GeneID 213), aldolase B (ALDOB; GeneID 229), cytochrome P450, family 2, subfamily E, polypeptide 1 (CYP2E1; GeneID 1571), fibrinogen alpha chain (FGA; GeneID 2243), transferrin (TF; GeneID 7018), and haptoglobin related protein (HPR; GeneID 3250). In some embodiments, a viral vector described herein includes a liver-specific regulatory element derived from the genomic loci of one or more of these proteins. In some embodiments, a promoter may be the liver-specific promoter thyroxin binding globulin (TBG). Alternatively, other liver-specific promoters may be used (see, e.g., The Liver Specific Gene Promoter Database, Cold Spring Harbor, http://rulai.cshl.edu/LSPD/, such as, e.g., alpha 1 anti-trypsin (A1AT); human albumin (Miyatake et al., J. Virol. 71:5124 32 (1997)); humA1b; hepatitis B virus core promoter (Sandig et al., Gene Ther. 3:1002 9 (1996)); or LSP1. Additional vectors and regulatory elements are described in, e.g., Baruteau et al., J. Inherit. Metab. Dis. 40:497-517 (2017)).

In some embodiments, a gRNA is introduced into a cell in the form of a vector. In some embodiments, the gRNA and a nucleotide sequence encoding a Cas endonuclease are introduced into the cell in a single nucleic acid (e.g., the same vector). In some embodiments, the gRNA and a nucleotide sequence encoding a Cas endonuclease are introduced into the cell in different nucleic acids (e.g., different vectors). In some embodiments, the gRNA is introduced into the cell in the form of an RNA. In some embodiments, the gRNA may comprise one or more modifications, for example, to enhance stability of the gRNA, reduce off-target activity, and/or increase editing efficiency. Examples of modifications include, without limitation, base modifications, backbone modifications, and modifications to the length of the gRNA. See, e.g., Park et al., Nature Communications (2018) 9:3313; Moon et al., Nature Communications (2018) 9: 3651. Additionally, incorporation of nucleic acids or locked nucleic acids can increase specificity of genomic editing. See, e.g., Cromwell, et al. Nature Communications (2018) 9: 1448; Safari et al., Current Pharm. Biotechnol. (2017) 18:13. In some embodiments, the gRNA comprises one or more modifications chosen from phosphorothioate backbone modification, 2′-O-Me-modified sugars (e.g., at one or both of the 3′ and 5′ termini), 2′F-modified sugar, replacement of the ribose sugar with the bicyclic nucleotide-cEt, 3′thioPACE (MSP), or any combination thereof. Suitable gRNA modifications are described in, e.g., Rahdar et al., PNAS Dec. 22, 2015 112 (51) E7110-E7117; and Hendel et al., Nat Biotechnol. 2015 September; 33(9): 985-989. In some embodiments, a gRNA described herein comprises one or more 2′-O-methyl-3′-phosphorothioate nucleotides, e.g., at least 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at the three terminal positions and the 5′ end and/or at the three terminal positions and the 3′ end.

In some embodiments, the gRNA comprises one or more modified bases (e.g. 2′ O-methyl nucleotides). In some embodiments, the gRNA comprises one or more modified uracil base. In some embodiments, the gRNA comprises one or more modified adenine base. In some embodiments, the gRNA comprises one or more modified guanine base. In some embodiments, the gRNA comprises one or more modified cytosine base.

In some embodiments, the gRNA comprises one or more modified internucleotide linkages such as, for example, phosphorothioate, phosphoramidate, and O′methyl ribose or deoxyribose residue.

In some embodiments, the gRNA comprises an extension of about 10 nucleotides to 100 nucleotides at the 3′ end and/or 5′end of the gRNA. In some embodiments, the gRNA comprises an extension of about 10 nucleotides to 100 nucleotides, about 20 nucleotides to 90 nucleotides, about 30 nucleotides to 80 nucleotides, about 40 nucleotides to 70 nucleotides, about 40 nucleotides to 60 nucleotides, about 50 nucleotides to 60 nucleotides.

In some embodiments, the Cas endonuclease and the gRNA are pre-formed in vitro and are introduced into the cell as a ribonucleoprotein complex. Examples of mechanisms to introduce a ribonucleoprotein complex comprising Cas endonuclease and gRNA include, without limitation, electroporation, cationic lipids, DNA nanoclew, and cell penetrating peptides. See, e.g., Safari et al., Current Pharma. Biotechnol. (2017) 18(13); Yin et al., Nature Review Drug Discovery (2017) 16: 387-399.

Small molecules have been identified to modulate Cas endonuclease genome editing. Examples of small molecules that may modulate Cas endonuclease genome editing include, without limitation, L755507, Brefeldin A, ligase IV inhibitor SCR7, VE-822, AZD-7762. See, e.g., Hu et al. Cell Chem. Biol. (2016) 23: 57-73; Yu et al. Cell Stem Cell (2015)16: 142-147; Chu et al. Nat. Biotechnol. (2015) 33: 543-548: Maruyama et al. Nat. Biotechnol. (2015) 33: 538-542; and Ma et al. Nature Communications (2018) 9:1303. In some embodiments, cells are contacted with one or more small molecules to enhance Cas endonuclease genome editing. In some embodiments, a subject is administered one or more small molecules to enhance Cas endonuclease genome editing. In some embodiments, cells are contacted with one or more small molecules to inhibit nonhomologous end joining and/or promote homologous directed recombination.

In some embodiments, genome editing systems described herein (or components described herein) can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intrathecal, intranasal, and intraperitoneal routes. Local modes of administration include, by way of example, intramarrow injection into the trabecular bone or intrafemoral injection into the marrow space, and infusion into the portal vein.

Administration may be provided as a periodic bolus (for example, intravenously) or as continuous infusion from an internal reservoir or from an external reservoir (for example, from an intravenous bag or implantable pump). Components may be administered locally, for example, by continuous release from a sustained release drug delivery device.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

### Targtets for Genomic Editingt

The disclosure includes compositions and methods related to genomic editing of a target gene (e.g., C3). In some embodiments, a target gene is C3 of one or more non-human species, e.g., a non-human primate C3, e.g., Macaca fascicularis C3, or e.g., Chlorocebus sabaeus in addition to human C3. The Macaca fascicularis C3 gene has been assigned NCBI Gene ID: 102131458 and the predicted amino acid and nucleotide sequence of Macaca fascicularis C3 are listed under NCBI RefSeq accession numbers XP_005587776.1 and XM_005587719.2, respectively. In some embodiments, a target gene is human C3. The amino acid and mRNA sequences of human C3 are known in the art and can be found in publicly available databases, for example, the National Center for Biotechnology Information (NCBI) Reference Sequence (RefSeq) database, where they are listed under RefSeq accession numbers NP_000055 (accession.version number NP_000055.2) and NM_000064 (accession.version number NM_000064.4), respectively (where “mRNA” in this context refers to the C3 mRNA sequence as represented in genomic DNA, it being understood that the actual mRNA nucleotide sequence contains U rather than T). One of ordinary skill in the art will appreciate that the afore-mentioned sequences are for the complement C3 preproprotein, which includes a signal sequence that is cleaved off and is therefore not present in the mature protein. The human C3 gene has been assigned NCBI Gene ID: 718, and the genomic C3 sequence has RefSeq accession number NG_009557 (accession.version number NG_009557.1). The human C3 gene is located on chromosome 19, and the genomic sequence of human C3 is shown below (from RefSeq accession number NG_009557.1):

| (SEQ ID NO: 1) | |
|---|---|
| 1 | gcagatagat tgattcagtc agtcaggtca aggttaactt gaattaatca gtaatagggt |
| 61 | ggaagaaggg gatggccttg ctgtgggttc tggagaaaaa ttctaggaaa gcagccacct |
| 121 | cagcctggaa ttagacgatg ggataggggt ttcccagctg ctcccaggcc tggctgcccc |
| 181 | tttgttgggg aaggggaggg atgggatata ggggacagtg agtgaactca ggcaggtgtg |
| 241 | agccgggggc atctgggtcc cccacccaga aatcattccc acttccttcc tcttattttc |
| 301 | tttctttttc ctgtcttgct ctgtcattca ggctgggggg cagtggtgca gtcatagctc |
| 361 | agtgcagcct ctaactcctc ctgcctcagc ctcccgagga gctgggactg caggcacgcc |
| 421 | accatgccct gctaattttt tttttttttt caattgtaga gacgaagtct cactgtattt |
| 481 | ctcaggctgg tctcgaactc ctggactcaa acaatgctct cacctcggcc tcccgaaagt |
| 541 | gctgggatta caagcacgag ccaccgcacc ctggcccctt ctcattttcc ccttgcaccc |
| 601 | cagctaggat tgccaaacag aatacaggac gctcagttac atttgaattt cagataaata |
| 661 | acaactactt tttcagtata tgtagcttcc agataaccca cgaatggtca gcccggttgg |
| 721 | ccacactctc cctccttgat tccgggaatg ctgggctggg tgggcctcaa aatggaaagt |
| 781 | accccaacac acacccagac ctccttctct ccctcccctg ctggctcatc cttgtgcact |
| 841 | atccccctcc caaacctctg gacaccaatg cacatctccc agaaaaaagt cacgaggttc |
| 901 | tgaagaattc ccggtctcat ctccctccct ccttccctcc cagtaggcta ccatctgctc |
| 961 | cagcctccaa ccccctcact tctcatcctg cccctcccct ctggtcactt cttggaggtc |
| 1021 | agggtagggc cagacccttt ccaggttcaa gtgattctcc tgcttcagcc tcccgagtag |
| 1081 | ctgggattat aggcacctgc caccatgctc agctaattct ttgttgttgt tgtttgtttt |
| 1141 | gttttgtttt gagacagagt ctcgctcttg tcgcccaggc tagagtgcag tggcacgatc |
| 1201 | ttggctcact gcaacctccg cctcccaggt tcaagtaatt ctcctgcctc ggcctcccca |
| 1261 | gtagctggga ttacaggtgc ccgccaccaa tcctagttaa tttttgtatt tttagtagag |
| 1321 | atggggtttc accatgttgg ccaggctggt cttgaactcc tgacctcagg tgatccaccc |
| 1381 | atctcggcct tccaaagtgc tgggatgaca ggtgtgagcc accatgccta gccagctaat |
| 1441 | ttttgtattt tttagtagaa acagggtttc accatgttag ccaggctggt ctcgaacccc |
| 1501 | cgacctccag cgatccccca gcctcagcct cccaaagtgc tgggatgaca ggcgtgagcc |
| 1561 | accacacctg gcccctctga gcctggtggc ttctaggcat cctggtttct ttaattgtca |
| 1621 | caacaaccag aactatcttc agtcgcattg tttagttgga ttaaccgagg ctcagagaaa |
| 1681 | agaggaaccc aggcttgccg ctagacagag gccagacagg aattccttct caaggttgtc |
| 1741 | aaaccacagt gccgaatgct tgagtctaga atgaaaccag gaaatggggt ggcttgagga |
| 1801 | gaaagtgggg gatagaagat ggaatggggc aattgggaga tccagtttct ttcctttttt |
| 1861 | taattttttt tttttttttt ggcaacaggg tctctctctg tcacccaggc tggagtgcag |
| 1921 | tggtgcaatc tcagctcact gcaacctctg cctcccggct tcaagcgatt ctcctgcctc |
| 1981 | agcctcctga gtagctggga ttacaggcac ccaccaccac gcctggctaa tttttgtact |
| 2041 | ttcagtaaag acggggtttc accatgttgg ccaggctggt ctccaactcc tggcctcaag |
| 2101 | tgatctgcct gcctcggcct cccaaagtgc tgggattaca gacgtgagcc actgcgcctg |
| 2161 | gcaaggggat gcagtttcaa aagctgaacc ccaattctgg agagcaagca ggtattttca |
| 2221 | ttctctctcc tcctcctcct cctcttccaa agagtgtgtc gcaatcagtg cagacagacg |
| 2281 | ccaggtttgt tctcatgctc cacgcctccc cctacccctg gcacggaaaa gaatgtggtt |
| 2341 | tacaggaaat cagagaaaac tccccattaa ccccttcagt ggggtttcag aaaccgcctc |
| 2401 | tccagggata agggggcccc acccacagac ccttctcctg ccctcaccat ccacctcgta |
| 2461 | tgcctgggca gcaatgctgc agaacgtcag aggaatgcca gttaaaatga caccggctgc |
| 2521 | cggggtgtgg tggctcactt ctataatccc agcactctgg gaggccgagg tgggcggatc |
| 2581 | acctgaggtc aggagtttga gaccagcctg gccaacatgg cgaaaccctt tctctactaa |
| 2641 | aaatacaaaa aataaaaaat aaaagaaaaa aaaaattagc caggtgtagt ggcgcatgcc |
| 2701 | tgtgatccca gctctttggg aggctgaggc aggagaatca cttgaaccca ggaggcagag |
| 2761 | gttgcagtga gctgagatgg cgccactgca ctccaccctg ggtgacagca caagactcca |
| 2821 | tttaaaaaaa caaaacaaaa caaaaaaaat gacaccaggg taccagtttt cacccataag |
| 2881 | gctggcaaaa atcttcaagt tcatcaacat gcccttgtga tgaggctgtg gaagaaactg |
| 2941 | acaattcatt tcatgcaggg ctcataagtg tgtaaatcaa tacaacttct gtgcagggga |
| 3001 | atttggcaat atctagcaag attaccagtg cattcagaga ttgacccaac atatttcctt |
| 3061 | tcattgcaac gacaactcta tgaagcaggt ggtaagggtt tccttttcca tgaacaaact |
| 3121 | gaggctcagg gcggtaatca gtagcttacc caaagatcac agctagtttc agagctagaa |
| 3181 | aataacgcag gttcaagctt attcactgca gagagcctgg tgtgaagcca cagatgtcag |
| 3241 | tctctccatc aagaagaggc tggtggctgg acacagcggc tcacgcctgt aattccaaca |
| 3301 | ctttgggagg ccaaggtagg tgggtcactt gaagtcagga gttcaagacc agcctggcca |
| 3361 | acatggtgaa accccttgtc cactaaaaat acaaaaattg ccagacgtgg tggtgctcac |
| 3421 | ctataatttc agccattccg gaggctgagg caggagaatt gtttgaaccc aggaggtgga |
| 3481 | gggtgcagtg agctgagata gcgccactgc cctccagcct gggtgacagg gcaagactct |
| 3541 | aaaaaaaaaa cactcaaaca aacaaaatat cccccaaaaa gtaggaggct ggttactttc |
| 3601 | tcacaatata acaagaggcc tgtaacctgt aagaatgagg cagttctttg ctcactgagg |
| 3661 | tgaaatagcc tctgaggtat attgttcatg aaaaaacgaa acaaaacgaa acccaagatt |
| 3721 | taactgaaga gaccaggaag aatagtatgt gctatgtgct gtccacaggg cacagtagtt |
| 3781 | cacaccagca ctttgtgagg ctgctgcggg aggatcactt gagcccagga gttcaagact |
| 3841 | ggactgggca acatcgtggg acccccatct ccacaaaaat aaaaaaatta tccgggcatg |
| 3901 | gtggcggcca cccgtagtcc cggctacttg ggtggttgag ccaggatgat cacttgaccc |
| 3961 | caggaggttg aggctgcagt gagctgtgat tgcaccactg caatttagcc tgagtgacag |
| 4021 | aatgaaaaaa aaattttttt aaaggaaaac acaaaaagaa tatgctgtca acagggatgg |
| 4081 | gaggaagacc acctttactg ctatacacat ttgtaccttt tagatgttga tcaatatgaa |
| 4141 | tatattatac acacagacac acacacagac acacacacac acacaaacaa tacaatttaa |
| 4201 | tatcctaaga ggatattgac attagacagg tacaaaagct ctagaaatga ggactttcct |
| 4261 | cagtgatgac ttttttcacc accaaagtca ctcaggcatc ctgacaaggg taagtgaggg |
| 4321 | gagcctcctt ggaaaataaa ctcacttgga tagtgaactc ctgcacatac ctcaaagccc |
| 4381 | atctgaaatg tcccctccta caggaagttt tccctgaccc tccaagaagc agagttctat |
| 4441 | ttcactgggg aaaacatttc ttcttcttct tttttttccc tgccctgcac atgagctaga |
| 4501 | aaacatttca tgaaactggg agtttctgtg ctgggctctg tccctccccc attctacttc |
| 4561 | ccctccctca gcatggaagc ctctggaagt ggggctctga ctcccagcct acagagagat |
| 4621 | tcctaggaag tgttcgactg ataaacgcat ggccaaaagt gaactgggga tgaggtccaa |
| 4681 | gacatctgcg gtggggggtt ctccagacct tagtgttctt ccactacaaa gtgggtccaa |
| 4741 | cagagaaagg tctgtgttca ccaggtggcc ctgaccctgg gagagtccag ggcagggtgc |
| 4801 | agctgcattc atgctgctgg ggaacatgcc ctcaggttac tcaccccatg gacatgttgg |
| 4861 | ccccagggac tgaaaagctt aggaaatggt attgagaaat ctggggcagc cccaaaaggg |
| 4921 | gagaggccat ggggagaagg gggggctgag tgggggaaag gcaggagcca gataaaaagc |
| 4981 | cagctccagc aggcgctgct cactcctccc catcctctcc ctctgtccct ctgtccctct |
| 5041 | gaccctgcac tgtcccagca ccatgggacc cacctcaggt cccagcctgc tgctcctgct |
| 5101 | actaacccac ctccccctgg ctctggggag tcccatgtga gtggttatga ctctacccac |
| 5161 | aaacagggct ggttctgggg tggaagcaga catttggggg tccaggtccc tgtagaattc |
| 5221 | agggtgcatt tgggtgtttg tggattcagg ggttagcagg ttgggaatga ttatatatat |
| 5281 | ttgggctgcc tgtgagtttg ggtgtttgtg gttgggtgtt tgtggaatcc aggtatcatg |
| 5341 | gaattggagt ttatatacat ttgggctgcc tgtgagtttg ggtgtttgtg gttgggtgtt |
| 5401 | tgtggaatcc aggtatcgtg gaattggagt ttatatacat ttgggctgcc tgagagtttg |
| 5461 | ggtgtttatg ggttgggtgt ttgtggaatc caggtatggt ggaattggag tttgggatgt |
| 5521 | ttctagaatt gaggtcatct gttggtttag ggtgtatgtg gtgttcattg atggtgcggt |
| 5581 | tgggggtgtt tggagactcg gaggtttgga ctttacaaga tttgggagtt tgcagcttgg |
| 5641 | ggacttgcaa ttttcagtgt gggtttaaag attggctact tcgggttcat gtatagttgg |
| 5701 | ggcatttgga attgattgta tttattagga ctggggtgtt ggaggtttag gctgggtttg |
| 5761 | gggtgctcta agatttgagg tttagaggtt ttggcgtatg tgggtttggg taggtagagt |
| 5821 | tgagggtgtc cgggagtttg agtgtttaca tatttggagt gtttagagag gtagaggttt |
| 5881 | agggtttggg gcatgtgtgg gtttaggcga ttgtgggtct ggaagtccag agacttggag |
| 5941 | gagttgctga cgctggttgg aaggttcagg gtttggtggg atgtgtggcc ccctcgttgc |
| 6001 | ccaggctttc aaaggccagg cccagctggc tgagagtggg agtcatggtg gctgctgtcc |
| 6061 | tgcccatgtg gttgagacgg tggcagtgcc cagagaagat aatggcattg gcaagtgcgc |
| 6121 | cggcagtcac tggatcctct ccaggaccag aggctggggc acacagcctg ccaggcgctg |
| 6181 | actccagtga ggactggcgt ctcacatccg tggaatgaca agcccactcc cgtgccccac |
| 6241 | tccgacaggt actctatcat cacccccaac atcttgcggc tggagagcga ggagaccatg |
| 6301 | gtgctggagg cccacgacgc gcaaggggat gttccagtca ctgttactgt ccacgacttc |
| 6361 | ccaggcaaaa aactagtgct gtccagtgag aagactgtgc tgacccctgc caccaaccac |
| 6421 | atgggcaacg tcaccttcac ggtgagtgca gactggcgca ggacccggct gacacccaca |
| 6481 | gccacgccca ctccccccct cctcctgagc ccctcccctt ctgtcttctc cctttctaag |
| 6541 | ccctgccctt ccctgagact ccaccccttc ggagtcgcct ctccttctaa gcccctccct |
| 6601 | tctctgagac tccacccctt ctgagtctcc tccccttata agcccctccc ttttctgaga |
| 6661 | ccccccccca ccccttctga atctcctccc cttctaagcc ctgaccttcc ctgagacccc |
| 6721 | accccttctg agactcctcc ccttctgagt ccctcccttc cctgagaccc caccccttct |
| 6781 | gaggttcctc cccttctctg agactccacc ccttctgagt ctcctccccc tctaagtccc |
| 6841 | tcccactgaa ttccttttcc aagcccctcc ccctcgaagt ctcctcttct gaactcctcc |
| 6901 | cctcttagtc tccatcactt tctaagttcc ctcacctgag tccctccccc tttctgagcc |
| 6961 | cctcccatgt cagccccttc cctttctgag tccccgcccc ttctgagccc ctcctcctat |
| 7021 | aagctctctc ctccttgtga gctcttcttt ttgagttccc tccctggtcc cccctctccc |
| 7081 | ctcgcacctc cttcacatgc ccctccctcc ccaaaacggc cacctcggaa gaccaagaat |
| 7141 | aatgggcagg caaggaggga cccagcccaa gatccggaag ctggaccgtg ggcatggggc |
| 7201 | cttggaacag acccctgaca atgccctgcc cacgcctaga tcccagccaa cagggagttc |
| 7261 | aagtcagaaa aggggcgcaa caagttcgtg accgtgcagg ccaccttcgg gacccaagtg |
| 7321 | gtggagaagg tggtgctggt cagcctgcag agcgggtacc tcttcatcca gacagacaag |
| 7381 | accatctaca cccctggctc cacaggtgag gctgggggcg gctggagagg gcggggcacc |
| 7441 | ggcgtgggcg ggctagggtc tcacgaggcc tctttgtctc tccccagttc tctatcggat |
| 7501 | cttcaccgtc aaccacaagc tgctacccgt gggccggacg gtcatggtca acattgaggt |
| 7561 | gccagccaga gggggcccca ggggaagcag gggcacaggc ttaggagagg caaagagatc |
| 7561 | gccagccaga gggggcccca ggggaagcag gggcacaggc ttaggagagg caaagagatc |
| 7621 | gagagagaca gagaaagaca caccggaagg ggtgcagtgg cagagacaca gaggcaaaga |
| 7681 | gatatgcaga cacacaccca cacaacacac acacatacag cacacaacat gcacacacac |
| 7741 | agcacacaat acacacacag aggcaaagag atatgcagac acatgtgcac acacaatgca |
| 7801 | cacacacaat gcaacacaca caaacacaca acatacacga ccacacaaca cacacaacac |
| 7861 | aacacacaac acaatacaca cagcacaacg tgcatgacca cacacacaac acacaacaca |
| 7921 | cacaacacaa tacacaacat acacaaccac gcaatacaca caaaacacac acaacacaac |
| 7981 | acaacataca taaccacacc acacacaaca cacaaccaca caacactatc acacaacaca |
| 8041 | cacaaacaca cacaacacac aacacacaca acacacacaa aacacaacac acacacaaca |
| 8101 | tacacaacca cacaacacac aaccacacaa catacacgac cacacaacac agtgcacaca |
| 8161 | aacatagcac acacaacaca caacccaaca cacaaccaca caatacacca tatggcgcgc |
| 8221 | acacacacac acacacacac acaggctgag agacaaggtg gagatccagg gagaccccag |
| 8281 | ggagcagtgc aggtgtccgt ggattctgct ttcagttaaa cccctgatca cttcacctcc |
| 8341 | ctgagcctca gttaccttat ctgaatatcg ggatcatgac ggataattgt atgtcatcta |
| 8401 | ttctaccgac ggcagccaga ggacgcctgt gagcacctga gtcagggccc atccctgctc |
| 8461 | tgcctacagc cctccatggc tcccaccttc ctatgcgtca aagcccaagt cctccctgca |
| 8521 | gtccacaagg ccctgcacac cttgccctgt cccttccctg ccctcccctc ctccctctct |
| 8581 | ccccctcgtt cactcttctg gagccacacg ggccatcctc cctgttcctc caacacccag |
| 8641 | gtgcagtcct gccttggcgc cttggcacgg gctgtgccct cttctcaaga aaaccctctt |
| 8701 | cttccaaata tccacacagc ttgttctctc tcctccttta agtctttgct caaatgtcac |
| 8761 | caatgtctca attttacaat gaggtctctc tgagtaacct ataaagtcgc aaatacccac |
| 8821 | cctgagcgtc ccccctcccc gctacacaca ctcctccttc ctgccatgtc ctgcaaatga |
| 8881 | gatttattca tttgataatt gcttctccca tcgcctcgcc ctctattgaa cctaaatccc |
| 8941 | tccaggaagg aattgttatg tttgttgagg gttttgtcac ctgaactcag cacaatgctg |
| 9001 | gtatatagtt gggtttaata aaaaacttac tggaagaagc gagaaggatg ggaggagaga |
| 9061 | aggggaagga gggtgttctc atagaattat catgaggatg tgttgaaatc atacaaggct |
| 9121 | aggtgcagtg gctcacactt gtaatcccag ctgtttggga ggccaaggcg ggaggatcgc |
| 9181 | ttgagcccaa gagtccaaga ccagcctggg caacacagcc agaccctgtc tctacaaaaa |
| 9241 | agaaaagtta aaaacaaaca aaaaaacagc tgtgtgtggt ggtgcttgct tgtggttgca |
| 9301 | gctaccccag gaggctgagg caggaggatc acttgagccc aggaattcca ggctgcagtg |
| 9361 | agccgtgatc gcaccactgc actccagcct gggtggcaga gtgagaccct gtctcaaaaa |
| 9421 | ataattgggg caaatgcaat ggctcaagcc tgtaattcca acatttcggg aggcagaggt |
| 9481 | gggaagactg ctcgaggcca agagttcaag accagcctgg gaaagctagg gagactacat |
| 9541 | ctctacaaaa aaaatgtaaa aattatctag atttagggat tgatgtggtc tgtggggaac |
| 9601 | agagaccaca catctcttgt aaaggcacaa cagttgccca gctccaatta gatgtctcct |
| 9661 | gctaaccaga gtacactatc cacagaaatt tccttgtttc caacagaagc tagaaaaaca |
| 9721 | gatttttggc caggtgcagt ggctcactcc tataatccca gcactttggg aggtggaggc |
| 9781 | gggcagatca cgaggtcagg agatcgagac catcctggct aacacggtga aaccccgtct |
| 9841 | ttattaaaag tacaaaaaaa aaattagctg ggcgtggtgg cgggcacctg tagtcccagc |
| 9901 | tactcgagag gctgaggcag gagaatggtg tgaacccggg aggcggagcc tgcagtgagc |
| 9961 | cgagatctcg ccattacact ccagcctggg cgacagagca agactccgtc tcaaaaaaaa |
| 10021 | aaacaaaaaa aacaaaaaaa aaacagattt ttatatgttt taattcctaa agccagctca |
| 10081 | cggccttcag atatgccact tgcctgatcc ctgttacctc tgtacaattt cttttaaact |
| 10141 | tatttattca ttcattcatt cattattatt atttttgaga cagggtctca ttctgttgcc |
| 10201 | caggctagag tgcagtggca caatcacagc tcactgcagc attgacctcc tgggcccaag |
| 10261 | ctgtcctcct gtctcagcct cctgggtagc tgggaccaca gacgtgcgcc accacatcca |
| 10321 | gctaatttta aaaaattttt gtagagatgg agtctcccta catttcccag gctggtcttg |
| 10381 | aacccttgac cttgagcaat cttcccactt ctgcctctca aagtgctggg attacaggct |
| 10441 | tgagccattg cgctcgccct aatacattat tttttgagat ggggtctcgc tctttcaccc |
| 10501 | agactggagt gcagtggtgc aatgatgtct catgatgttt aaatgttggc agcaaatgaa |
| 10561 | atgacactac tagttattag tattcagaga gacactgaaa aaatgagccc ctactcatat |
| 10621 | gaactatgtc ccaagccaac acagtaggtg ccattataat ctcctgtttc aagatttgca |
| 10681 | cattgagcac agagaggtta ggtaacttgc ccagggtcac acagcttgta agtggcacag |
| 10741 | tagagattga aacctaaggt tgactgactc cggtccttgt tctttttttc gagacagact |
| 10801 | ctcactctgt ctcccaggct ggagtgcagt ggagtgatct tggctctctg caatctccgc |
| 10861 | ctcccgggtt caagcgattc tcccgcctca gcctcctgag tagctgggat tacgggtgcc |
| 10921 | taccaccatg cctggctaat ttttgtattt ttagtagaga cagggtttca tcacgttggc |
| 10981 | caggctggtc ttgaactcct gacctcaggt gatctgcccg cctcagcctc ccagagtgct |
| 11041 | gggatgacag gcgtgagccg ctgcgcccac ctgggtccct gttcttaacc acagtagaca |
| 11101 | ctgtgcacag agaatgtcca gacacaggtc ggggagagct gagaggctaa gcccagcctc |
| 11161 | cgaagagcca ctttatcctc tatccttccc tcctgcctcc cacagaaccc ggaaggcatc |
| 11221 | ccggtcaagc aggactcctt gtcttctcag aaccagcttg gcgtcttgcc cttgtcttgg |
| 11281 | gacattccgg aactcgtcaa gtatgtcagg ttcttgagga gggggctcag ggctccccta |
| 11341 | tccccggaga gggagcaggg gggctccgag gcctgagaga ccactcatcc gccctcctca |
| 11401 | cagcatgggc cagtggaaga tccgagccta ctatgaaaac tcaccacagc aggtcttctc |
| 11461 | cactgagttt gaggtgaagg agtacggtaa gaggaggagg ggctgggggg agtcagtgcc |
| 11521 | cagaacgcct ggcccagcgc cggccccacc aacgccatct ctcccccagt gctgcccagt |
| 11581 | ttcgaggtca tagtggagcc tacagagaaa ttctactaca tctataacga gaagggcctg |
| 11641 | gaggtcacca tcaccgccag gtgagggact gggggtgggg ccaggtaaga gccaggtgag |
| 11701 | ggaccaggtg aagaccaggt gggggactgg gggtggagtc aggtgggggg ctggagatgg |
| 11761 | gaccaggtgg ggggctgggg gtggagtcag gtggggggct gggggtgggg aaggtggggg |
| 11821 | gctgggggtg gggcaaggtg aggggctggg ggtgggacca ggtggggggc tggggggtgg |
| 11881 | agtcaggtgg gggctgggag tggggaaggt ggggggctgg gggtggggcc aggtgagggg |
| 11941 | ctggaggtgg gaccatgtgg ggggtgggag tggggcaagg tggggggctg ggggtggggc |
| 12001 | caggtgaggg gctggaggtg gggccaggtg agaggccagc agtgggttgg gggctccagt |
| 12061 | cttcagcaca ggcaggagaa gctgggggag atcccattct ccaggaggga tggacctgaa |
| 12121 | gccctccttg tctgtcccgt aggttcctct acgggaagaa agtggaggga actgcctttg |
| 12181 | tcatcttcgg gatccaggat ggcgaacaga ggatttccct gcctgaatcc ctcaagcgca |
| 12241 | ttccggtacc atagacggag gccgctttga tccctgcccc agtccccgcc acctctgagc |
| 12301 | ccgctcccct ctctgagccc tcctctccct tctcagattg aggatggctc gggggaggtt |
| 12361 | gtgctgagcc ggaaggtact gctggacggg gtgcagaacc cccgagcaga agacctggtg |
| 12421 | gggaagtctt tgtacgtgtc tgccaccgtc atcttgcact caggtgaggc ccagtctgaa |
| 12481 | ggccaggctc aggaccacca agtgggccgg tctgagaggg gagaccaggt cagaagagaa |
| 12541 | agcctagtct aaggagggag gctcagagtg aaagtggggt tcagtctgat ggggtaggcc |
| 12601 | cagtctgaga ggggaggccg agtatgaaga tggattccag cctgatgggg ggaggcaggg |
| 12661 | ccagtataaa ggtggggtcc gggctgatgg gggcacaggc ccagtatgaa gtctgtgtcc |
| 12721 | agtctgatga gggaggcagg gccagtataa agatgggtcc agtctgatgg gggaggcagg |
| 12781 | gccagtataa aggtggggtc cggtctgatg ggggtcacag gcccagtatg aagtctgtgc |
| 12841 | cagtctgatg gaggaggcaa ggccagtata aaggtggagt ccagtctgat ggggggcaca |
| 12901 | ggcccagtat gaaagtggac tctactctga gggaggaggt ctagtctgaa gttggggtcc |
| 12961 | attctgaggg aggaggtcta atcctgaggg gtggcccaga agcctacact cacagctggt |
| 13021 | cccctcaggc agtgacatgg tgcaggcaga gcgcagcggg atccccatcg tgacctctcc |
| 13081 | ctaccagatc cacttcacca agacacccaa gtacttcaaa ccaggaatgc cctttgacct |
| 13141 | catggtgaga cccggggcgg gaaggggtcc cactcctccc ttcggggaca ccggccacag |
| 13201 | ccctgagcct gcctgaactt cccccacctg caccccacat cacaggtgtt cgtgacgaac |
| 13261 | cctgatggct ctccagccta ccgagtcccc gtggcagtcc agggcgagga cactgtgcag |
| 13321 | tctctaaccc agggagatgg cgtggccaaa ctcagcatca acacacaccc cagccagaag |
| 13381 | cccttgagca tcacggtgcg tctgggccca gcctcggaac cccatcactg ggaagacggt |
| 13441 | acaggggttc tggtgtttgc acagtggggt cctgtcattt gcatacagat attctcatct |
| 13501 | gcatagagag gttctctcct gcgcagaggg gtcctgccat ttgcatagag atactctcat |
| 13561 | ctgcatagag gggttctgtc ctgcacagtg gggtcctgcc atttgcatag acattctcat |
| 13621 | ttgcctagag gggttctgtc ctgcacagtg gggtcctgcc gtctgcatgg aggggtccgc |
| 13681 | agtttgagga aacaggaatc ttcctcttgc atgccctgct ccttccactt acacggagag |
| 13741 | gcgctccatc cacgcacagt ctttccactc ccatggggga aggagcctga atctcacaag |
| 13801 | gagggttgtg tagtgtttgg gacaggccca ttgttgtgag gtggtctcag ttctcctggc |
| 13861 | ttctgtgcac gtggctctgt tgcccctcac tgggagggaa gcaagtctca tgacagctgc |
| 13921 | ggaggttgca gatggcctcc cagtccctct gcagctccca ggctgcgcac cccacttacc |
| 13981 | cctccctgtg ctcagcatgt gcgtgaattt ccggtggcta ccatgagaaa tggccacagc |
| 14041 | ctagtgatct aaagcaacac acatttatgg gtctatagtt tgagaggtca gaagtcctgg |
| 14101 | ctctggggga aagttcgctc ccttgctttt tccagtgtcg ccagggcacc ctaaaggcct |
| 14161 | ggctcatggc cccttcctcc acctttaaag gcagcagcat agcatcttcc agtgtctctc |
| 14221 | tttctctctg tctctgtctc tcctttctcc cctgcccctg cttaataaag acccttatga |
| 14281 | ttacattagc tccacctaca taatccagga taatgattcc atctccagat ccctaactta |
| 14341 | atcccatctg caaagcccct tttgttaaga aaggccacca attcccaggt ctcagggatt |
| 14401 | cgggtgtggg tatcctcggg cggcgaccag caggcatccc tctttcccca cccaggtgcg |
| 14461 | cacgaagaag caggagctct cggaggcaga gcaggctacc aggaccatgc aggctctgcc |
| 14521 | ctacagcacc gtgggcaact ccaacaatta cctgcatctc tcagtgctac gtacagagct |
| 14581 | cagacccggg gagaccctca acgtcaactt cctcctgcga atggaccgcg cccacgaggc |
| 14641 | caagatccgc tactacacct acctggtccg tggccacctg gaaacctcag cccccgcctc |
| 14701 | ctccttgttt cttccgcacc cctgggactc cttcccccat cccggatccc tccctgcgtt |
| 14761 | ccctgccact caccctcccc agcctgatgc cagcctgtcc ccccagatca tgaacaaggg |
| 14821 | caggctgttg aaggcgggac gccaggtgcg agagcccggc caggacctgg tggtgctgcc |
| 14881 | cctgtccatc accaccgact tcatcccttc cttccgcctg gtggcgtact acacgctgat |
| 14941 | cggtgccagc ggccagaggg aggtggtggc cgactccgtg tgggtggacg tcaaggactc |
| 15001 | ctgcgtgggc tcggtaagtg tgccctgggc tcgctcgccc cctctccctc tccctactcc |
| 15061 | tctctctctc tctctctccc tgtctcctct ctctctctct ctccctttct ccttttctct |
| 15121 | ctcctttctc tctcttctct tcctctccct ttctctcctc cctctctgtc tctcaactgt |
| 15181 | ctctcttttt atctctcttt ccctctctct acatctctct ttccctctct ctttatttct |
| 15241 | ctttccttct ctctctccct ctctcgatct ctctttctct ccatctctct ccttttctct |
| 15301 | ctccctctct ctctcctttt ctctctccct gtctctttcc ctttccctct ctctcccctc |
| 15361 | tctttctctc cctctctctt tccctctccc tctctctctc cctttctctc tctccctctc |
| 15421 | tctccttctc tctccctctt tctctccttc tctctttccc tctctctctc cctctctctt |
| 15481 | tccctctctc tccctctccc tttctctccc tctttccctt tccctctctc ccccctcact |
| 15541 | ctccctctct ctgtctctcc gtctctctcc ctctctccct gtctctccgt ctctctccct |
| 15601 | gtctctccct ttctctctct ctcccgccct ctctccctct ctctccctcc ctctctccct |
| 15661 | ttctctctct ctccctctct ctccccctcc ccagccccac ggctcccccc aacctttctg |
| 15721 | tctttccact ctagcccagc acccactcca tcccaggcac tcctctctcc cagggctgac |
| 15781 | ttctttcggc gtctccaccc tccccacagc tggtggtaaa aagcggccag tcagaagacc |
| 15841 | ggcagcctgt acctgggcag cagatgaccc tgaagataga gggtgaccac ggggcccggg |
| 15901 | tggtactggt ggccgtggac aagggcgtgt tcgtgctgaa taagaagaac aaactgacgc |
| 15961 | agagtaaggt aagggccagt gacccaaggc tgctgagaag aggcggaggc acggagctgg |
| 16021 | ggctggggga ggtgggtggg actggagagg gcagtgcagt ggggggcatg cgctgaaagc |
| 16081 | agagatcgga gcagaccaga cacagggatg gttgaagctg aagatgggaa tgaggttgga |
| 16141 | catgggttcc aattggggat ggtcctgaga attggacttt tttttctgtt tgtttgtttg |
| 16201 | tttttgagac agagtctctc tctgtcacca ggctggagtg cagtggcaca atctcggctc |
| 16261 | actgcaacct ctgcctccca ggttcaagcg attctcctgc ctcagcttcc ctagtagctg |
| 16321 | ggactacagg tgcccatcac cacgcccagc taatttttgt atttttagtg aagacggggg |
| 16381 | tttcaccatg ttggccagga tggtctcgat ctcttggcct tgtgatccac ccgcctcgac |
| 16441 | ctcccaaagt gttgggatta caggcgtgag ccactgcgcc cggctgagaa ttggacactt |
| 16501 | tcaactgggg ccctgagagg ctggtggcag cacacccagg gtcattcagt ggggaaggtt |
| 16561 | tccggagtag ggacgaagat ggagatgggg ttggcttggg atcaggagtg aggatgggaa |
| 16621 | tgcagatgga atcagagggg aaatggagat aagatttgga atggaggcca ggtgcggtgg |
| 16681 | ctcacgtctg gaatcccagc actttgggag gtcaaggtgg gaggatcact tgaggccagg |
| 16741 | agttcagacc agcttgggca acatggcaag accccatctc tacagaaaaa attttaaaat |
| 16801 | agctgggcat gatggcgcat gcctgtagtc ccatctgctc aggaggcaga ggtgcgagga |
| 16861 | ttgcttgagc ccaggaattt gaggctgcag tgagctatgc ctgcaccact gcactccagc |
| 16921 | ctgggagaca gtggaaaatc ccaacttaaa aaaaaaaaaa aagaatggaa agaaaggagg |
| 16981 | aaaaaaaaag aagagagaga gaaacagaga gaaagaaaaa gaaaggagat aaagaggaag |
| 17041 | ggagggaggg agtgaagaat gaaggaagga aagaaggaag gaaggaagga gggaaggagg |
| 17101 | gaaggaaagg gggagcaaag gaaggaggaa aggaggaatg gagggaggaa gggagggaga |
| 17161 | ggaaggaagg gaaagaaaga agacagaaag aaaagaaaaa gaaggccggg catggtggct |
| 17221 | cactcctgta atccctttgg gaggccaagc actttgggag gccaagacag gcgaatcatt |
| 17281 | tcaggtcagg agttcgagac cagcctggcc aacatggtga aatcccgtct ctactaaata |
| 17341 | tataaaaatt agctgggcat ggtggcatgc acctgtagtc ccagatactc gggaggctga |
| 17401 | ggcaggaaaa ttgcctgaac ctgggagttg gaggttacag tgagcggaga tcacaccact |
| 17461 | gcactccagc ctgggtgaca gagcaagact ccatctcgaa agaaagaaag agagagagtg |
| 17521 | agaaagagaa agaaaaagag aaggaaggag agagaaggaa ggaaggaaag agaaagagaa |
| 17581 | aggaagggca gaagcaggaa tgggggagat gagagtggga cagggtgggg tcatttggga |
| 17641 | agagatacac aggtgcatat gtgggggatc ccaattgtca gcctggcctc cctgcgtccc |
| 17701 | gccaccccta tgccccccgc agatctggga cgtggtggag aaggcagaca tcggctgcac |
| 17761 | cccgggcagt gggaaggatt acgccggtgt cttctccgac gcagggctga ccttcacgag |
| 17821 | cagcagtggc cagcagaccg cccagagggc aggtgaggtc gccaccaggg gccggtgcag |
| 17881 | ggacagacag cacctccacc tcccagatgc tgggagcaga gctctggaaa ccgggggcct |
| 17941 | gggttcaagc cccgcctcca ccaccaccta gtaaatccct cccctctgag cctcagtttg |
| 18001 | ctcttccatc aaatgggagc aggaacaccc ccacctcaca cgatcgtgag gggtgaaccg |
| 18061 | aggacaccta gtaggtgcct catccatctt cttctcggtc cgcctgccct gcagaacttc |
| 18121 | agtgcccgca gccagccgcc cgccgacgcc gttccgtgca gctcacggag aagcgaatgg |
| 18181 | acaaaggtgg gagcctttcc tacccactcc tgcccccgag ccccacccca ggagacccca |
| 18241 | gcccggccgt gcaggagcca gagagggagg aggggaggcc ctggcggcgg ggaagtcctc |
| 18301 | cctggggtcc gtcccgcgtc cctcctgctg ccggcccccg gctgagggtg tggcctgggg |
| 18361 | gaacacgtgc tcccgcagtc ggcaagtacc ccaaggagct gcgcaagtgc tgcgaggacg |
| 18421 | gcatgcggga gaaccccatg aggttctcgt gccagcgccg gacccgtttc atctccctgg |
| 18481 | gcgaggcgtg caagaaggtc ttcctggact gctgcaacta catcacagag ctgcggcggc |
| 18541 | agcacgcgcg ggccagccac ctgggcctgg ccaggagtag gtcccacggg gtggggacag |
| 18601 | ggggaggggg ccgtctgatg ggggaggaga ctcctgtctg aggagggagg atgccctgtc |
| 18661 | tggtgggggt ggggctggag gaggccgctg tctgaggggg gaggaggccc ctgtctgagg |
| 18721 | gggcaggagg tccctgtctc aggggggagg aggcccctgt ctgaggaggg aggaaacctc |
| 18781 | cgtctgagga gggaggaggt ccctgtctga ggagggagga ggccttgagg ggggaggagg |
| 18841 | tccccgtctg aggagggagg aggcctctgt ctgaggagag aggaggtacc tgtctgaggg |
| 18901 | gggaggaggc ctctgtctga ggggggagga tgcccctgtc tgagggggta ggaggaggcc |
| 18961 | tctgtctcgg ggggaggagt cccctgtctg aggagggagg aggcctctgt ctgagggggg |
| 19021 | aggatgccgc tgtctgagag ggtaggagga ggcctctgtc tgttgggaga ggaggcccct |
| 19081 | gtctgagggt gatgccgatg aggtgatgcc ctgccagcgt gaggtagaga agacccaggt |
| 19141 | ctgaagaggg gaggatcaag tcagagaagc gtagatgccc atctgagatg gaggaggctc |
| 19201 | ccgtccgagg ggaggggaca ctcctgtctg gaagggacag aggccttcag atgaggagcc |
| 19261 | aggaggccca ggcctgaggg aggagaaggg cctagtctga tggggagaag ggcccttgcc |
| 19321 | tgaaggcaga gcagtttcct gcctgggaag gtcatcccag ccccacccat cagtctgaat |
| 19381 | tggacatcac cagtgcccag gacattggag gtctgaggga aaagtctaga aagatgatgg |
| 19441 | ggctggtcac acactaatta ccaatgggaa agctaaggtg agttccaagt ttggcttcac |
| 19501 | cagagaaaac taatttgtgt ggcattccag aaagacctgc caaactcgat gagtgaacag |
| 19561 | gcagcccttc ttcattcatg catgcattca gtttttgaat caggtgagac tttagatctc |
| 19621 | acgtgaaata agtcttaagt gaaacaaaga gaaatttatc ttataataag agaaaattgg |
| 19681 | ccgggcatgg tggctcacac cggcaatcgc agcactttgg gaggccgagg tggatggatc |
| 19741 | acttgaggtc aggagttcaa gactagtctg gccaacatgg tgaaaccccg tctctactaa |
| 19801 | aaatgcaaaa atagcctggc gagctggcag gcgcctgtaa tcccagctac tcaggaggct |
| 19861 | gaggtgggag aatcgcttga acctggtagg tttaggttgc agtgagctga gattgtgcca |
| 19921 | ctgcactcca gcctgggcaa cagagcaaga ctccgtctca aaaacaaaac aaaacaaaac |
| 19981 | aaaaaaagaa aggaaaaaga aaattggccg ggcacggtgg ctcacacctg taatgcccac |
| 20041 | actttgcgag gccgagaagg gtggattgct tgagtccaga aatttgagac cagcctgggc |
| 20101 | aacatggcag aaccccatat ctacaaaaat aaaataaaat aattagccgg gtgtgggggt |
| 20161 | gcacacctgt agtcccagct actcaggagg ctgaggtggg aggatcgttt gaacccagga |
| 20221 | gatggaggcg tcaatgagcc aaaatcacac caccgcactc cagcctgggc aacagagcaa |
| 20281 | gaccctgtct caaaaaagaa aaaaaaaaaa agagagagaa aagaaaagaa aatgaaaaga |
| 20341 | aaaaattcaa gcaaatttag aatgatctcc ttcacaaaga ggcgatagtg tgagggtcac |
| 20401 | tgggaaaatt agacaaaaag tctggtctac tgaaatatgg tttacatcca catggatggt |
| 20461 | gggctgtact tttctccaga attgtgtaat tcctttggcc cattgggggt cagaaaaaga |
| 20521 | atggctaaat gttactatcc caagacactt ggattgatta ttccagagtg tgagtaaatt |
| 20581 | caggtatctc ttttaggaat tccatctact ttgggctggg cttagtggct cacacctgtg |
| 20641 | atcccagcac tttgggaggc tgaggcagcg ggatcgcttg agctctggag tttgagagca |
| 20701 | gtctgggcag cgtagtgaga ctttgtacgg acgaaaactt tttttttttt ttttgagatg |
| 20761 | gaatcttgct ctgtcaccca ggctgaagta cagtggcaca acctcggctc accgcaacct |
| 20821 | ccacctcatg ggttcaagcg attctcctgc ctcagcctcc tgagtagctg agattattat |
| 20881 | tatttgtttt tttgagatgg agtctcgctc tgtcacacag gctgcagtac agtggtgcaa |
| 20941 | tcttggctca ctacaacctc cgcctcccgt gttcaagtga ttctcctgcc tcagcctccc |
| 21001 | aagtagctgg gattacaggc acctgccacc acacccagct aatttttgta tttttagtag |
| 21061 | aaaagaggtt tcaccgtgtt ggccaggctg gtgtcgaact cccaaccttc ggggatctgc |
| 21121 | ccgcctccgc ctcccaaagt attgggatta caggcatgag ccactgtgcc tggctgaaaa |
| 21181 | atattaaaat atatatattt tttaagggat tccagctact ttgttgttat ggagatccag |
| 21241 | aacccaatta aagcctgtct atcatgtttg aggaaagtgc agtttgagtc aaagcctagt |
| 21301 | ccagtccaat ttcatttact tgctggtagt gtcaagctgt ttttgtttat ttatatattt |
| 21361 | atttagaggc aggatcttgc tctttcgccc aggctggagt gcagtggtgc gatcacagct |
| 21421 | cactgcagcg tcaacctctt gggctcaagg agtccttctg tctcatcctc agccttctga |
| 21481 | gtagctagga ctacaggtgc atgccagcat gcccagctaa tttttaaatt attatttgta |
| 21541 | gagagagggt ctcagtgtgt tgcccaggct ggtctcaaac tcctgggctc aagccatcct |
| 21601 | cccaccttgg cctctcagag cgctgggatg atagcaccac atccagccta tcgagatttt |
| 21661 | ttttgtgttt ttttctttgt tttttgtttg tttgtttgtt tgtttgagag ggagtctcgc |
| 21721 | tctgtcgcca ggctggagtg cagttgcgca gtctcggctc actgtaacct ccgcctcctg |
| 21781 | gattcaagag attctcatcc ctcagcctcc cgagtagctg ggattacagg cgcatgccat |
| 21841 | cacacccagc taatttttgt attaggtggt ttttaaaggc caccgcttct tcagtgttct |
| 21901 | gcaccaggtc tgggaatgtt ctcagctcac ctagtcatgt tcagaatgga caaatccctc |
| 21961 | agaggaagca gacacggttt ctcgggacgg tgatccttta gagccacatg cacatgcttg |
| 22021 | ctttctttta ttattatctt tttttgagat ggagtctcac tccgtcaccg aggctggagt |
| 22081 | gcagtggcat aatcttggct cactacaacc tctgcctccc gggttcaagc gattctcctg |
| 22141 | cctcagcctc ccgagtatct gggactacag gtgcccgctg ccaagcctgg ctaattttca |
| 22201 | tatttttagt agaggcgcgg ttttgccaca ttggccaggc tgtctcgaac tcctgacctc |
| 22261 | aagtgatcca cccgcctcgg cctcccaaag tgctggaatt acagatgtga gccactgtgc |
| 22321 | ctggccaaat gctttcgttt ctttaaaaat caaagggaaa ggaatgacta taatccagtc |
| 22381 | tgcattgtat atgtccttat accagtacat ttgtgggata taatttttag ttctttttat |
| 22441 | ggagaagaag ttcccaaggc agatgtgtct ggggctcgtg aaaattcatc ctgaagtcct |
| 22501 | ccatgtccgg gatgtatttc actgctagga atccctcctg ggcagaggta ggatctaaag |
| 22561 | gtgtgaccgc tgaggaagta ggtcggctct ctttttgttt gttttttgtt tttgttttca |
| 22621 | gatggagtct gtctctgtcg cctgggctgg agtgtagtcg tgtgatctca gctcactgca |
| 22681 | acctccacct cctgggttca agtgattctg ctgcctcagc ctccacagta gctgggatca |
| 22741 | caggcacgcg ccaccacacc cagctaattt ttgtgttttt agtagagatg gggtttcacc |
| 22801 | atgttgtcca ggctggtctc aaagtcctga cctcaagcga tccacccacc tcagcctccc |
| 22861 | aaagtgctgg gattacaggg gtgagccacc gtgcccagcc ttaatttttg tatttttagt |
| 22921 | agagatgggt ttcaccatgt tagctaggct ggtctccaac tcctggcctc aagtgatcca |
| 22981 | cctgccttgg cctccctaag tgctgggatt tcaggcatga gccatggcaa ctggcctgct |
| 23041 | ctgttctaaa tgcagatcta aaccccctgc aggtaacctg gatgaggaca tcattgcaga |
| 23101 | agagaacatc gtttcccgaa gtgagttccc agagagctgg ctgtggaacg ttgaggactt |
| 23161 | gaaagagcca ccgaaaaatg ggtaaggccg gggtaccccc ggtacaaccc accccagagt |
| 23221 | cagaccgttt aatttgcatg cacctgctat ctctggtctt ctctggaatc acagtgcaac |
| 23281 | cccacagccc aacctagaaa aatcaggaat tgggtgacct acatggaggc acccccagac |
| 23341 | ccttccagcc tgtcccttgg ggtccctctg caccagttct tcccctctac caccctgcta |
| 23401 | gatgacatct cctaataccc caacctcttc tccatccaga atctctacga agctcatgaa |
| 23461 | tatatttttg aaagactcca tcaccacgtg ggagattctg gctgtgagca tgtcggacaa |
| 23521 | gaaaggtgag agaggatgct ggctggtccc cgggaggcag ggaccccagg gtgtctgagt |
| 23581 | gtcatctcat tttatccaaa ctcaatcaac cctatgtttc ttggcacttt attctctgcc |
| 23641 | ctggttacca cagaggtgtt gttaccagga actgtgggaa tccttagttc ctgtctaact |
| 23701 | tggaagaaag aattcagcca agagtcacat agcaagggtt aagtagcaga gtttattgaa |
| 23761 | ggaagaaaca gctctgggct ggtccccctg gaaaaatagt agtagcaatg cttatttaaa |
| 23821 | gagacagggc cagcctcgat ggctcacacc tataatccca gcactttggg aggctgaggc |
| 23881 | aggggaatca cttcaggtca ggagttcaag accagcctgg tcaacgtggt gaaaccccgt |
| 23941 | ctctactgaa agtacaaaac aattagccag gcagggggtg gcgggcgcct ataatcccag |
| 24001 | ctactcggga ggctgaggca ggagatttgg ttgaacccgg gaggtggagg ttgcggtgag |
| 24061 | ctgagattgt gccactgcac tccagcctgg gcaacaagag caaaactcct tctctaaata |
| 24121 | aataaaaagt gaccgtatgc tctgaaagac gacacagaca tggctgctca acagaacgag |
| 24181 | ccagcagcag atactgctgg tagactcttt ttatgagact cttacatgat ttttcgtgaa |
| 24241 | ggggcgtgag tgggtgtcac ttgtaagcat gttttgggag gtctctttgg gcgagcaggc |
| 24301 | tctgtggctg taggtactag catgcacgtg gcatgtctca ttagcatcga aaatctccac |
| 24361 | ccagaggtgt gttttttact atgataatga gcaaaacaca actctagggt gttttcggag |
| 24421 | cagtgcacat gctcatcatc ggggaaaatc cctagcaaag ttatttccag ctaggacctg |
| 24481 | ataagtcccc ttcagggcca gaggacccca accacaaggc catgtgtagc taaagtagcc |
| 24541 | atcgtccttt tcgctgactg ccagtgagca gcgctgtcag taggcagcct gtctgggact |
| 24601 | tcttttccca gaaagctccc ctgcctgctc atttccgcct atctgcctac tctaacagtg |
| 24661 | tcaaaagcta gacagggtgg gggtacagtc tctaaaattg atgcttttct ttctttcttt |
| 24721 | tgtttttgag aaggagtctc actcggtcat ccagccataa tttatatggt ttattataat |
| 24781 | ttataataaa tttaattata atatttattt atatatttat taattgtaat gtttataatt |
| 24841 | ataatatata attatatatt acataatata tttcatatct acatatcaca tattacatat |
| 24901 | gcaatatatt atataccaca tattacatat ataacatacc acatattaca tatataatat |
| 24961 | atcatatatt atatattaca tatataatat atcatatatt atatattaca tatataatat |
| 25021 | atcatatatt atatattaca tatataatat atcatatatt atatattaca tatataatat |
| 25081 | atcatatatt acatatatta tatattacat atataatata tcatattaca tatattatat |
| 25141 | attacatata taatatatca tattacatat attatatatt acatatataa catatatatt |
| 25201 | acatatatca tattacatat atcatatatt acatatataa tatatcatat tacatatata |
| 25261 | tcatatatta catatataat atatcatatt acatatatat catatattac atattacatg |
| 25321 | taatatgtta tattacatat aatatatatt gcatatcaca tatataatat gttatatgtt |
| 25381 | gcatattaca tatataatat attatatatt gtatattaca tatataatat atatgtaata |
| 25441 | tatacatatt acacatgtaa tatattatgt aaacatataa tatgtattat aatttataag |
| 25501 | aaatttaatt ataatataat ttaatgaatt ataataaacc ataattcatt ataatttaat |
| 25561 | acattataat aaaccataat ttattataat ttaattttgt tgtaatgtat aattataatt |
| 25621 | tactactaat atgtcatttg ttattgttga catgttaaca tatataatgt atattttatt |
| 25681 | agatatataa tataaatgat gtatcattta ttattgatta catatctata attataccat |
| 25741 | atcataactt attacaaaac attctattta atttaaatat acccaaaata gtatcatttc |
| 25801 | aacattttgt aaaaagttgc aaaaccacaa cccactaata atgtgactat aaccttttaa |
| 25861 | tatttgataa taatctacta gtatatcaaa attactgatg atatatttta cttctgtttg |
| 25921 | cactaagtct tcaaaatcca gcatgtgttt tacaattcag tgcatctcat ttaggatact |
| 25981 | agattttctt tctttttttt ttttgataca ggagcttgct ctgtcaccta ggatggagtg |
| 26041 | cagtggtgta aacaggatgc taagttttct ttttttagta gagacagggt gtcaccatgt |
| 26101 | tggccaggct ggtctcaaac tcctggcctc aagcaatctg ccttcctcag cctcccagag |
| 26161 | tgctggaatt acaggcgtga gccaccgcgc ccagcgcagg atgctaggtt ttcactggaa |
| 26221 | atactttgat ctgtatttta ggtttcataa aatttacagt tgaaaaggta gattctcagg |
| 26281 | ccgggtgcaa aggctcaagc ctgtaatccc attactttca gaggctgagg ccggcaaatc |
| 26341 | atttgaggtc ggagtttgag accagcctgg gcaacatggc aaagccccgt ctctacaaaa |
| 26401 | aaaaaaaaga aaagaaaaga aaagagaaag aaaaggtaga tcctcatact caagtagttg |
| 26461 | caaaaatact taaacgtttt ccactcaatc atcattttta aaaaattaag atttaattca |
| 26521 | cttactatat gtcacccttt taaaatgtac aactcaggtc gggcacggtg gctcacacct |
| 26581 | gtaatcccag cactttggga ggcccaggca ggcagatcac ctgaggtcag gaggtggaga |
| 26641 | acagcctggc caacatggtg aaaccctgtc tctactaaaa atacaaaaaa ttagcaggac |
| 26701 | atgcgggtgg gtgcctgtaa tcccagctac tcaggaggct gaggcaggag aattgcttga |
| 26761 | acccaggata tagaggttgt agtgagccaa gatcacgcca ctgcactcca gcctgggtga |
| 26821 | cagagcgaga ccccatctca aaaaataaat aaataaaaaa taataaaata tataattcag |
| 26881 | tggtgtttca tatatttaaa atgagcatca gttgtttgtt ttgtttcatt gggtttggtt |
| 26941 | ttacagacag gatctcactc tgttgcccag gctggagcac agtggtgcga tcatagctca |
| 27001 | ctgcagcctt gaactcctgg gctcaagcaa tcctcctgcc tcagcctccc aaagtgctgt |
| 27061 | gattacaggc atgagccacc gcacctagct agatcatcag gtttaaagtt taagtctgaa |
| 27121 | ttaaattaaa tacatttaaa tacaagtaca tcaaataaaa gtacaaatcc agtttctcac |
| 27181 | tcaggcaaac cccatttcaa gtgctcagcg ctcccccaca gcttggggct accatatcag |
| 27241 | acaagcagat atattttgga gatttctctt cctccctaca cgtagatctc tgagtcaaac |
| 27301 | tacaaacaga atgtaaatca ttaaatagtg gtaactccgg ccaggcgcag tggctcacgc |
| 27361 | ctgtaatctc agcacttggg aggctgaggc gggtggatcg tgaggtcaag agatcgagac |
| 27421 | catcctggcc aacatggtga aaccccatct ctactaaata tacaaaaatt agctggacat |
| 27481 | ggtggtgcgt gcctgcagtc ccagctactc gagaggctga ggcaggagaa ttgcttgaac |
| 27541 | ccaggaggcg gaggttgcgt tgagccgaga tggcgccact gcactccagc ctggcgacag |
| 27601 | agtcttgctc tgtctcaaat aattaataat aataataata ataataataa taataataat |
| 27661 | aaataatggt aactcccagc caccaccatc atcatctgtc atttgtcgcc attgacagcg |
| 27721 | tttagttcac aggcttcagc aaagacaggc tgagttaggg agagctcctg cggagtggac |
| 27781 | taagagctga gacccaggag cctggccttg tccactcccc gaccttgaca ctccgtgttc |
| 27841 | tgtctctgcc cgagcaggga tctgtgtggc agaccccttc gaggtcacag taatgcagga |
| 27901 | cttcttcatc gacctgcggc taccctactc tgttgttcga aacgagcagg tggaaatccg |
| 27961 | agccgttctc tacaattacc ggcagaacca agagctcaag gtgggtcccg gggtggcaga |
| 28021 | ggcttcttgg aggctgccag ggggtaggta gcctgttgca cacacacttg cccggatcct |
| 28081 | ttctctccct ggcaggtgag ggtggaacta ctccacaatc cagccttctg cagcctggcc |
| 28141 | accaccaaga ggcgtcacca gcagaccgta accatccccc ccaagtcctc gttgtccgtt |
| 28201 | ccatatgtca tcgtgccgct aaagaccggc ctgcaggaag tggaagtcaa ggctgctgtc |
| 28261 | taccatcatt tcatcagtga cggtgtcagg aagtccctga aggtcgtggt gagtgcttgg |
| 28321 | ggcacccaca aacccttgtc cttcagagag ggctcctggt cttcgtacta ttgactcagg |
| 28381 | ttggagatcc aggctctgag acactaagaa tcatagtgtc cagcttagga aatttggaag |
| 28441 | tcccagaatt tcagaagcag agccaggatt ggggtaaagt gagtgagatg accccaggct |
| 28501 | tagaatttta ggtggtgcca aaaacctcgt cgaccatcac caatcaataa tttttttata |
| 28561 | ctcgatttga aattttttat ttatttattt atttgtttgt ttattttttt gagacagagt |
| 28621 | ctcactctgt tccccaggct ggagtgcagt ggcgcgatct cagctcactg caatatccgc |
| 28681 | ctcccgggtt cacgccatcc tcctgcctca gcctcccgag tagctgggac tacaggcgcc |
| 28741 | agccaccacg cccggctaat ttttttgtat ttttagtaga gacagggttt cactgtgtta |
| 28801 | gccaggatgg tctcgatctt ctgacctcgt gatccaccca cctcggcctc ccaaagtgct |
| 28861 | aggatcacag gcacgagcca ccgcgcccgg caatgctagg gtgatcctaa ggacagtgcc |
| 28921 | ctgctgacca tctgtgtgtc tgtctgttct tttattcatc caacgactcc ccccacctct |
| 28981 | aacactgcgt agccggaagg aatcagaatg aacaaaactg tggctgttcg caccctggat |
| 29041 | ccagaacgcc tgggccgtgg tgagtcggct gcagggggag gggctgaggg gctggcaggg |
| 29101 | taaggggggt aaatgacctg ggtttagtga ggtaggatag ggcgggaggg agctagagcc |
| 29161 | atcggtatct ctcactcacc ctgcagaagg agtgcagaaa gaggacatcc cacctgcaga |
| 29221 | cctcagtgac caagtcccgg acaccgagtc tgagaccaga attctcctgc aaggtgagac |
| 29281 | acccttgacc ccgaccccat gggtcccagg agggcatgga tggagccaaa ttccatctca |
| 29341 | ttctggaggt gtttaacccg cacctttctc ttccccttca gctagaacag cccatctgtg |
| 29401 | atctgttttc cctcttttac attttttttt tttttttttt ttgagacaga gtctggctct |
| 29461 | gtcacccagg ctggagtgca gtggcgcgac ctcagctcgc tgcaagctcc gcctcccggg |
| 29521 | ttcacgccat tctcctgcct cagcctcccg agtagctggg actacagcca cccgccacca |
| 29581 | cgcccggcta atttttttgt atttttagta gagacagggt ttcaccgtgt tagccaggat |
| 29641 | ggtctcgatc tcctgacctc gtgatccacc cgcctcagcc tcccaaagtg ctgggattac |
| 29701 | aggcatgagc cattatgccc ggcctaaaaa tttttttaac catacagata ttatttgcta |
| 29761 | tgatcggttt tatagaagcc tccagatagc atttagttca gcaaagagct ttcgctgata |
| 29821 | catcagttta ttttaatttt tctagacctt ctgtgcttct tagatgggaa accagcttaa |
| 29881 | atgagactca atagcctgta atcccagcac tttgggaggc cgaggcaggc agaccacctg |
| 29941 | aggtaggagt ttgagaccag cctggccaac atggtgaaac cctgtctcta ctaaaaatac |
| 30001 | aaaagttagc tgggcgtggt ggcacatgcc tgtaatccca gccactcggg aggctgaagc |
| 30061 | aggataatcg attgaacgtg ggaggcgtag gttgcagtaa gccgagatca ggccactgca |
| 30121 | ctccagcctg ggcggcagag caagactttg tctcaaacaa aaacaaacaa acaaacaaac |
| 30181 | aaaaagacaa gcaacatagt acaagagcag aaattctgga ggtcatttct tgccccagga |
| 30241 | gggaagactg gagaaagaaa gggacttgca acctgtaagc tataaggctt tggggcaaga |
| 30301 | gccttggttt tttcaccttt ggtaggggta gaataatagt atctacctcc aagggttggt |
| 30361 | gtgatgattt tttttttttt tttgaggcgg agtctcactc tgtcgccagg ctagagtgca |
| 30421 | gtggcgtgat ctcggctcac tgcaacccca gcctcccggg ttcaagtgat tcttgtgcct |
| 30481 | cagcctccca agtagctggg actacaggcg cccgccacca tgcccactaa tttttgtatt |
| 30541 | tttagtagag acggtgtttc accatattgg tcaggctggt cttgaactcc tgacctcagg |
| 30601 | tgatccaccc accccagcct cccaaagtgc tgagattaca ggcttcagcc acggcgccca |
| 30661 | gcctcgttga ctattaagtg agacactcta tggtattctc ttagaacagt ctggaaagta |
| 30721 | acattaagcg tgatataagt attcctgaat attgttactg gaattatttt actgctggtg |
| 30781 | aaatgagacc caaggaccag ggtgcccctg tgaagcacct cccactccta acagtgcaga |
| 30841 | cccccgaaca gccactcagc catgcagcct cccctccccg cagtcacatc ctccccagtc |
| 30901 | ctcgcctgtc cctaacccct tggccctggc tggttgggag gctggaaccc ttttcacgcc |
| 30961 | accccaaggt gggtcaccca cctggcttga gcaacgtcct cttcccacct gctgcaggga |
| 31021 | ccccagtggc ccagatgaca gaggatgccg tcgacgcgga acggctgaag cacctcattg |
| 31081 | tgaccccctc gggctgcggg gaacagaaca tgatcggcat gacgcccacg gtcatcgctg |
| 31141 | tgcattacct ggatgaaacg gagcagtggg agaagttcgg cctagagaag cggcaggggg |
| 31201 | ccttggagct catcaagaag ggtgggctcc ctgcccctct tggagacccc agggacccct |
| 31261 | ttccgagcgc atccctcccc taagatccca cctcatctca agaccacgcc ctcccctgag |
| 31321 | gctccacctt ctctcctagc cactcccctc atttgaggcc ccacctcttc tcaaggctac |
| 31381 | gccctctgag gccctgactc ctcccaggcc aggcttttca tgagaccccg cctctcctca |
| 31441 | aggccatgcc catcccctga gggcccccca cctcttctca aggccacgcc ctctgaggcc |
| 31501 | ctgactcctc ccaggccagg ctcttcatga gaccccgcct ctcctcaagg ccatgcccat |
| 31561 | cccctgaggg ccccccacct cttctcaagg ccacgccctc tgaggccctg actcctccca |
| 31621 | ggccaggctc ttcatgagac cccgcctctc ctcaaggcca tgcccatccc ctgagggcct |
| 31681 | cccacctctt ctcaaggcca cgccctctga ggccctgact cctcccaggc cagaatctcg |
| 31741 | agaccctgcc tcttttcaag gccacgccca tcccctgggt ccccacatct tctcaaggcc |
| 31801 | acacccttct gtgaggcgcc acctcctgtc ccagccactc tcatctgagg ccccacgtcc |
| 31861 | tctccaggcc atgcctcttc cctgagactc caccccctct ctgagagccc tcccctccct |
| 31921 | gaaagccccc caccctcaat atccttctcc tctctgaatc ccttgtcctc ttgagaactt |
| 31981 | ttccacctcc tcgttctgat cccccaccct ctttgagtcc ttcccttttt aaggtcccct |
| 32041 | cctcccagaa cccctccgcc accctgagcc cctgtcccct ctctgcaccc cgcccctgcc |
| 32101 | ctttctggcg tgccccctct gctcagcccc ggctcttttg ggggttcctc tctcttctct |
| 32161 | gcagggtaca cccagcagct ggccttcaga caacccagct ctgcctttgc ggccttcgtg |
| 32221 | aaacgggcac ccagcacctg gtgagtccca acagccagct caggccatgc atactcccca |
| 32281 | ccctcaaccc ccagcagggc ccggaccctg gccaggggtg gtcccttagg ccagccttgc |
| 32341 | ccaaacagcc ctggacctgc agagtccagg caagcgctgg ctgagtggcc ggcggtcatt |
| 32401 | aagcatcctt aagcacggac cgcatacaac agctgggtcc tggggcctgg gaaggcaaac |
| 32461 | caggcaaact gggccaggcc ctggtccctc ccccacgctc attggctggt tgacatggca |
| 32521 | gtctctggat ctcagagccg attggctcat gctctgtgcc cactccaggc tgaccgccta |
| 32581 | cgtggtcaag gtcttctctc tggctgtcaa cctcatcgcc atcgactccc aagtcctctg |
| 32641 | cggggctgtt aaatggctga tcctggagaa gcagaagccc gacggggtct tccaggagga |
| 32701 | tgcgcccgtg atacaccaag aaatgattgt aagaggctgg gatttagggc aaaatggaag |
| 32761 | agaggggctc ctgagtctcg caggatgaac acgagagaga gccccacctc catgtgccca |
| 32821 | ctgcccaatt ccctttgcaa agattgggct ggggggtggg ggcaggcaga tatatgagcc |
| 32881 | agaggcgtca ctccagcatt gcaaaaacca gagacctgcg aagcccagcg caaaatgaag |
| 32941 | agacacggcc cctcgctcag aaattattaa gaatttcatt aaaccaagtg caggggtcct |
| 33001 | gcctgggaat ccctttctca cattcaatcc atcaacacct gcattctccc atgatgttat |
| 33061 | aagaatcacc tccttctctc catccttatg gccagcccct ggtccaagca acactctccc |
| 33121 | cgcccctcct tatttggaga ccttgtagaa accacctcct ggtcatcatc ctggtggcct |
| 33181 | cccacttttg ttggctctca gacactcacc acatagcagt tggggtgatt ttttcaaatc |
| 33241 | cagctggatc agttcttaga aagtcccgtg gctccccctg tggcacttaa acacaaaact |
| 33301 | ccttcgagca ctggttctcg aagtgtgatc ctcagaccag cggcagcaac agcacccatg |
| 33361 | acttactaaa aatgtgcatt ctgtggctgg gctcgacggc ccatgcctgt aatcccagcg |
| 33421 | ctttgggagg ccgaggcagg aggatggctt gagcccagga ggtcgaggct gcagtgagcc |
| 33481 | atgatcatga cactgcactc caggctgata acagagtgag accctgtctc aaaaacaaaa |
| 33541 | catattctga gaccggaccc cagactcact gaatcagaaa ttctaggggc aggacccagg |
| 33601 | aatctgaggg gtgtgagtgt gtgtgtgtgt gtgtgtgtgt gtgtgtgtgt gtgtgtgtgt |
| 33661 | gtttgagatg gagttttgct cttgtcaccc aggctggagt gcaatggccc gatcttggct |
| 33721 | cactgcaacc tccacctccc aggttcaagc aattctccta cctcaacctc ctcagtagct |
| 33781 | gggattacag gtgcccgctc caccatgccc agctgatttt tgtattttta gtagagacgg |
| 33841 | ggtttcacca tgttggccag gctggtcttg aactcctgac ctcaggtgat ccgcccacct |
| 33901 | tggcctccca aagtgctggg attacaggca tgagccaccg cgcccggcct aggaatctga |
| 33961 | gtttttaaaa gtgcccgcat tcctccaggt gatgctaatg tgtgcttgag atggagaatc |
| 34021 | actgcctcag tctcaccttt caggcttcca gacttccagc ctttcttttc tttccaggct |
| 34081 | ccatccattg ataggagcct tgctctattg ttctacaggg cctttgcaca tgctgtttct |
| 34141 | gccacctagt atgctaatcc ctgccgtctg tgagagttga ctccctcagg gacacttttt |
| 34201 | ctgacctccc caactgggtc acactcccac agttcattat cgctgcgatg tcctctttcc |
| 34261 | cttgcacaga actcatccac ttataagtat atatctcttg gctgggcgca gtggctcatg |
| 34321 | cctgtaatcc cagcactgtg ggaggccgag gcaggtggat cacctgaggt caggagttcg |
| 34381 | ggaccagcct gaccaacagg ggaaacccca tctctactaa atacaaaaaa attagcttgg |
| 34441 | tgtggtggtg catgcttgta atcccagcta cttcggaggc tgaggcagga gaattgcttg |
| 34501 | aatccaggag gcggaggttg cagggagtcg agattgcgcc attgcactcc agcctgggca |
| 34561 | acaagagcaa aactgtccca aaaaaaaaaa aaaaaagtgt atatctcttg aggagctgga |
| 34621 | tggaccatgt ccatcttccc tactagacaa aagctctgtg agggctagag cctgtgtctg |
| 34681 | gttttacaat ggatcagacc gttgtaccca ttgtacattg cacattgtac attgacattt |
| 34741 | gcagaaggaa caaattgttg catgaattaa tactaagaag tttgaccttc ctagggtagc |
| 34801 | ggggtaacac ctagaagaga ctcagccctg cccagacccc ctgattctga atctgcaagg |
| 34861 | ggggatgact gccatgtgtg gacacaccgg tgaccccatc cttgctttct gctctctatc |
| 34921 | tcagggtgga ttacggaaca acaacgagaa agacatggcc ctcacggcct ttgttctcat |
| 34981 | ctcgctgcag gaggctaaag atatttgcga ggagcaggtc aacgtaagtg ccctccatct |
| 35041 | tcccacccta ccctacctta cccgatgcag agcacagcca ccttggagag tgagaggttg |
| 35101 | ccttcaggga atttgcagct ctcccagtgc aataacagac atcactgcag tcatgttaat |
| 35161 | agctaacatc ttttgagcac ttaactcatc taatacagac ccgccctcta atagtttcac |
| 35221 | atgttaagtc tcataatcct tttagcagcc tgaaaggtaa gtcactctta ttatccccag |
| 35281 | tttgcagatg agaaaactga ggcacaaaga gatcaaaggt ggggattctt tctgtctgcc |
| 35341 | ttacaatttt cagagggttt tcagcccatt tccaaaagtg ctttctacat cagtgctaca |
| 35401 | tgatcagtac agttgcgtac ttgctacttc cttaaagaaa acttgggata cagagctaag |
| 35461 | actatttcct tagtccagag gatctttcag gtgattttca aagggatccg tgactccaaa |
| 35521 | caggaaacgg tgaacactgt tggctcatca ctgtctcttt ttcctctggt tttgattctg |
| 35581 | aagcagggaa gcttggaaag atgggccgct gagagtctgg aatgcctttg tctgctttat |
| 35641 | tgtggttgtt tgtttgtttg tttatttttt gtgatggagt ctcactctgt cgcccaggct |
| 35701 | gcaatgcagt ggcatgatct cagctcactg caccctttgc ctcccaggtt caagggactt |
| 35761 | tactgtttca gcctccagag catctgggat tacaggcacc cgccaccata cccggctaat |
| 35821 | ttttgtcttt ttagtagaca tgaggtttca ccatattggc caggctggtc tcgaactcct |
| 35881 | gacctcaggt gatctgcctg gcgtggcctc ccaaagtgct gggattacag gcatgagcca |
| 35941 | ctgcacccag cctaattgtt gtatttttag tagagatggg gtttcaccat gttggccagg |
| 36001 | ctggtttcga actcctgacc tcaagtgatc cacccacctt agcctcccaa agtgctcgga |
| 36061 | ttacaggcgt gagtcactgc acctagctga tcgtggggtt ttgagtgggt tgtttaacgt |
| 36121 | ttagctttcc aagtgggaag cccaggattc caccctcagc tagtggcttc tcccccctta |
| 36181 | ggaaaagaga tggaggggag gggccagtga agagaaaaac aaacacaggg ctgttgcctc |
| 36241 | taacacccaa gagggaccaa ggcagagaga gagagagaga gagagagaga gggagggagg |
| 36301 | gagggaggga gggagggagg gaggtaggta gagagagaga gagagagaga gaggagaggt |
| 36361 | ggggtcagac aaatctgact tcaaatcctg actcatgggc acttccaccc ttgagcctca |
| 36421 | ctcaggatgt gcatctgtaa attggggata ataaataacg atctctgtat ttttaggcct |
| 36481 | ctgagttgtc ccagatataa cacacatgtg acccagatta tacaaaaatt gatggggaat |
| 36541 | ttatgtgcag gcaccaaggc atcaaataga gatgaaggtg gcctcaggga ctctgccagg |
| 36601 | atgctttgct cctctctccc gtgatcttca ttccgttctt ggccaataat tcagttcagg |
| 36661 | cagaatatgg ctgccttcct tagagaaaat atcagatcaa ggttagggcc gccatattcc |
| 36721 | caggaaagga ctctgattgg ctcagcctgg gtcagatgac tatatctgga ccaatcagct |
| 36781 | aaggacagga agtaggtctc agggggcaga catggctgtt tccactgtgg ccacgtgaat |
| 36841 | ggaagggaga agaagttctt acaaaaggag tggatgtcag agaggcaaat gggcaggaat |
| 36901 | aaaagagatt tgtttctgct acaacatagc aacattgtag cagagtatag cacaggctgt |
| 36961 | gaaaccagac tcctggggtc aagagtgtgc tgtaatccca actactcaag atgctgaggc |
| 37021 | aggagaatca cttgaaccag ggaggtggag gttgcagtga gccgagattg cgccactgca |
| 37081 | ctccagcctg ggcaacacag caagactcct tttcaaaaaa aaaaaaagtg tgctataact |
| 37141 | agcttgctgg agcccagtgt taaatttcca ggaatttttc aagctggtca ttaaatacaa |
| 37201 | ttattattaa aaactaaata ttaggccagg cacagtgagc ctgtaatccc ggcactttgg |
| 37261 | gaagccaagg ccggcagatc acctgaggtc aggagttcaa aaccaccctg gccaacatgg |
| 37321 | caaaaccccg tctctactaa aaatacaaaa attagccggg catggtggag gggggcgcct |
| 37381 | gtaatcccag ctacgcagga ggctaaggca caagaatcgc ttgaacccgg gaggcggagg |
| 37441 | ttgcagtgag ccgagattgc gccatgcact ccagcctggg ccagagcgag actccgtctc |
| 37501 | aaaaaaaagg ccaggcgcgg tggctcacgc ctgtaatccc agcactttgg gaggccgagg |
| 37561 | tgggcggatc acgaggtcag gagatcgaga ccacggtgaa accccgtctc tactaaaaat |
| 37621 | acaaaaaatt agccgggcgc ggtggcggac gcctgtagtc ccagctactg ggaaggctga |
| 37681 | ggcaggagaa tcacttgaac ccgagaggcg gagcttgcag tgagccgaga tcgcgccact |
| 37741 | gcactccagc gtgggcgaca gagcaagact ccgtctcaaa aaaaaaaaaa agcaacaaca |
| 37801 | aaaaacccaa ccaaccaacc aaacaaacaa agttataaaa gttacagtta aataaattat |
| 37861 | attaaacaca aaggttagaa acactcaaac tcatcgcttc ctaaacgcct tactcccata |
| 37921 | atctatactc ttggggttac ttatgtctgt tggatctgta tagtgaaaat actatataat |
| 37981 | actgtggtac tgcaaagctc ttcccaactc tacattcaac gacaccatat tggtaggttg |
| 38041 | aaatcagtga tggaagtatt tacatcatgg aaatgagaaa acagtacaaa tcatgtcttc |
| 38101 | ccccatcccc agaaggctgt gtttggatcc taactctgcc acttatttcc taggtggtct |
| 38161 | ttgcaaaatt actgcatctc tcagggctca gtatgctcat caggttttat gagattaaat |
| 38221 | gtgtgggtat ctgaatgaca caaagtaagt gtgagctatg atgatgaaga agataaagat |
| 38281 | gatgatgacg atgatgatga tgactggatg aggtgttcac agtggtatac tgaatctggc |
| 38341 | gcatactagt ttatgagtaa caatttggag aatgtctccc caggactttg ttcagtgatg |
| 38401 | tcgcattgac accgtgaaat tggcccctgg tgggagtatt tacaccacag aaattgtaaa |
| 38461 | tcattataaa ccaaggatcc ctcaaccctc ccactggaga gctggctgtt aaacttttac |
| 38521 | cagcacacca cggggtacgt ggatttctcc agatacataa tagatatgca gcaacaaggc |
| 38581 | agctcatggt ggctaaaata tctgggaaat tctcaaaaat ggacaaatct aagacaggtg |
| 38641 | tgtcccaagg acagaaatcc ctgatgctca ggaagtgctg ctcgaatgat ccttactaac |
| 38701 | gtgacagcaa tgcccacatg accggagaat ctgatcctct ttctcataga gcctgccagg |
| 38761 | cagcatcact aaagcaggag acttccttga agccaactac atgaacctac agagatccta |
| 38821 | cactgtggcc attgctggct atgctctggc ccagatgggc aggctgaagg ggcctcttct |
| 38881 | taacaaattt ctgaccacag ccaaaggtga gggttggcct ggaggggtga agggagatgc |
| 38941 | atggctgaag ttcagggcgg gagatactga gctgggatgc atggctttta gctgagctgg |
| 39001 | gacagatgac cctaagccaa gctgagatgg atagtcctaa ggtatcaagc tgggatgcat |
| 39061 | aaccctgagc tgagctggga tgcacggctc taagttttcg caggtcctca ttgtaaacca |
| 39121 | cacgagaaag tttgttgcgt catttattca acaaatgcgt attaagcatt catttcaaag |
| 39181 | ggagaagtga gagttgatga aacaagagag gtaaggcagg agccaagtaa ttgagagcct |
| 39241 | cgaatgtcag ccaggacacc caaacaccag gaagtctagc atgcatctct ttctgagctt |
| 39301 | tctctgagcc atccccaggc tggacagagc agtgagcact ggggatgggg tatcttcttt |
| 39361 | gcagataaga accgctggga ggaccctggt aagcagctct acaacgtgga ggccacatcc |
| 39421 | tatgccctct tggccctact gcagctaaaa gactttgact ttgtgcctcc cgtcgtgcgt |
| 39481 | tggctcaatg aacagagata ctacggtggt ggctatggct ctacccaggc aagtgggccc |
| 39541 | acagccccta ggcacatgca tccctgtctc ctgcggcttc ccactggcct cctagagaag |
| 39601 | acactgaggc ccagcgaggc agttcttcat tcccacgagc cagtgtgatt gcagtggagt |
| 39661 | tgagaatcag tttttattac ttgcaaaccc atctataggt tctagaatac aatctgggta |
| 39721 | ctccaagctg tgtgttgagc cttcttcttg ccccaggtgt ctagatcatg ttctcagggc |
| 39781 | ccaggttcag gtctaagcct ctctctccac ctggtgggct ctagaccagg ttcccagttc |
| 39841 | tatctcacaa tcttaccctg tcttgctggt gggttctaga ccatgttccc agttctacca |
| 39901 | ggctcccaat gtcacattgc ctcactggcg ggctctatag tatgttccca gttaccctgg |
| 39961 | ggcattacgc aaaccctctt ctaggccatg gtttcagtaa cttcaggctt cagcaacttc |
| 40021 | aggctccagt tggcctcctt tctttctggt ggtctgtcac tcacgttctc agtgttacag |
| 40081 | tgtcactctt gggttgtaga ttatatgctc agtatcctct ggctacggtt tcattctgtt |
| 40141 | cttcatgagt gggttctaga catattctca gtgtctccaa gccctggtct aagactctct |
| 40201 | cctcttgatg ggtctagact gcatcctcag ggtcgctaga cattcagtct tacatttgga |
| 40261 | ctttctgatg gattctagac atgttctcag catctccaag tcctggtgta agtttctgtc |
| 40321 | tctcggagag ttctgaacat gtcctcagag tccagtgacc tccagttatc acccctgcac |
| 40381 | tctctagtag gttctaggcc acattttgat gtcccagctc tgatttgaac ctctttatcc |
| 40441 | cccactggat tctagccact ttcccaggct cccagatcac catctttctc tcttgtgggt |
| 40501 | tctaggccac cttcatggtg ttccaagcct tggctcaata ccaaaaggac gcccctgacc |
| 40561 | accaggaact gaaccttgat gtgtccctcc aactgcccag ccgcagctcc aagatcaccc |
| 40621 | accgtatcca ctgggaatct gccagcctcc tgcgatcaga agaggtacag tcacccagcc |
| 40681 | aagccctcct cactctggct gtctccccct acactagcca gggtttactg ggaagcaaga |
| 40741 | gggagggcca ggtgaccatc acaggcagca gaaggcttaa ttcccaacat gctctcttct |
| 40801 | ctcttttcac tctgcagacc aaggaaaatg agggtttcac agtcacagct gaaggaaaag |
| 40861 | gccaaggcac cttgtcggta aggaacagaa acccacacct gcctggccca tgcccctctg |
| 40921 | ccccagaggg accatctcct cttgtcccca gcagtcctag tcctgtgggc tgacattgtg |
| 40981 | tctcctctcc catcttacca ggtggtgaca atgtaccatg ctaaggccaa agatcaactc |
| 41041 | acctgtaata aattcgacct caaggtcacc ataaaaccag caccggaaac aggtaaaagg |
| 41101 | aatcaaggcc ttatctgtca ccttcctcct acccctcttc taatgtcttc cccgctcctg |
| 41161 | aatcaacaca caggtatacc ctctcccatc tttctctctt ctgtgtttct agaaaagagg |
| 41221 | cctcaggatg ccaagaacac tatgatcctt gagatctgta ccaggtaaga agctaggtca |
| 41281 | ccggggttca tcttggccat ccctctatct ctagcaagaa ttcttgcaaa taatatccat |
| 41341 | gatattcagt actttccaag tacactgtgt atctgatact gttctaagta tccaccatga |
| 41401 | ggtagacaac acagacagtc cttgctttgc atgttaatgt gagaccacag caatgaccac |
| 41461 | gtaagctgag actgtcaaag catcttagta atcaatggag gaaagtacac aatcattcca |
| 41521 | tgacctttaa agttttcttt ttttcttttt agagagatag ggtcttgctc tgtcagccag |
| 41581 | gctggagtgc agtggcacaa tcatagctca ctgtaacctc aaactccctg gctcaagcga |
| 41641 | tcctcctgcc tcagccactc aagtagctgg gactacaggc gtgtgccatg acacctggct |
| 41701 | gatttttatt ttttattctt tctagaggca gggcctcact gtgttgccca ggctggtctc |
| 41761 | gaactcctag ccttgagcat tcctctgcct tgggctgcca aagttttggg atcacaagca |
| 41821 | tgagccacta tgcccagcct aaatgtttct attacaacat ttaaaattat catactgcca |
| 41881 | gttataaaga tacagggaaa tggccgggtg tggcggctcg cgcctgtaat cccagcactt |
| 41941 | tgggaggctg aggcgggcag atcacgaggt caggagatcg agaccatcct ggctaacacg |
| 42001 | gtgaaacacc gtctctacta aaaatacaaa aaaattagcc gggcatggtg gcgggtgcct |
| 42061 | gtagtcccag ctacttggga ggctgaggca gaagaatggc gtgaacccag gaggcggagc |
| 42121 | ttgcagtgag ctgagatcac gccactgcac tccagcctgg gcgaaagagc aagactctgt |
| 42181 | ctcaaaaaaa aaaaaaaaaa aaaaatagaa taaaacaaaa taaagataca gggaaatgaa |
| 42241 | attcatagta agatgagtat ttgactacac cgtaatttaa aacattagaa cattgagatg |
| 42301 | caaggtgtat ttgttgtttt ttttttcctt tgtatgacac ttacggagag tactttagtt |
| 42361 | caaaaaaatg cttgccttct tctctttgta taatttacaa catggagtaa acatcttttc |
| 42421 | tatgccttag taccttgtct tgctcctttc taagtttgga tcagcttcca atattttatc |
| 42481 | ctttgagctt tccatgacac aaaattcctc caagagttcc tttaaagtga ctttgtattc |
| 42541 | tataatgtcc cttcctctgg gacatcttca tcctttttgt ccccatgacc ttccttattt |
| 42601 | atgctaatac atttgccttc cctgagttcc tctacactac ctatctctca aatggcagca |
| 42661 | gggtcaacat caccatagtc tgctattctt tgataactcc atttatgctg tctttgaagt |
| 42721 | tcacttctgg cattatcact tttcatttct ttgctgcatt tttatctttg ttggccagtt |
| 42781 | ccctcttttc gtgatacatt gttgtaaaat ctcatgggag ttagccacct ggagacaggg |
| 42841 | aggcaacaga actacacact ttgctgtctg tgcataaatt gaagagcaga agctcagtga |
| 42901 | ccaatcactg atggactttg aaaggagtga cagtaattgg ccctcaatta tgatgcttat |
| 42961 | cttttattta tgtcgtgatt tctagactga agagttagca acaaagttta taccatatgc |
| 43021 | aactactcgt gatcaatata ccaaggtact gaaaaagaac catgtcactg ggctactagt |
| 43081 | gttatttaac tgaatcatgc agagtgaggg ctgcctgtat tcttgccttg ttttctagaa |
| 43141 | ctgaagcatg gagggtcaaa taatgcatcc aatgttattt agagctggaa tttgaatcca |
| 43201 | tgcagttggg tgcagagtct gagctcttaa tcaccttgac cattacatta ccttgctttt |
| 43261 | tatttccttt ggggaaatgt ttcctaaaaa atgtaacgcc cctctgtgct gctatgtggg |
| 43321 | aatcagaagt ctcagtgcct gatcagacct ccttgtccag gaacagaccc ttggggctga |
| 43381 | cccctccttg ggacccaatg cccttctttc tgcactatcc aggtaccggg gagaccagga |
| 43441 | tgccactatg tctatattgg acatatccat gatgactggc tttgctccag acacagatga |
| 43501 | cctgaagcag gtatgaaggg ctcaggagct gggataagtg gaaaggagcc tgggttctgg |
| 43561 | aagaggctgc agggagagag gggtccagga gggatttttc acaggctcca cctttcccca |
| 43621 | gctggccaat ggtgttgaca gatacatctc caagtatgag ctggacaaag ccttctccga |
| 43681 | taggaacacc ctcatcatct acctggacaa ggtaaggctg catcatcctc ccctgggagg |
| 43741 | cttccagggg caccctgacc tctatctggc tggtctttct tttcctttca gcttttgtct |
| 43801 | ctgggtcaga ctaaccctgg gccagaggag acagggtctg tgctgctgag ttgtagggga |
| 43861 | aggagcttgt aaaataaggg ggtcaaccca gcatcttcta taaacatctc atcttctgac |
| 43921 | catttgcctc ctccaacttg ttatcagagt cttaaacaac cattgaaaaa aagccctttt |
| 43981 | ggtttttttg gttttttttt taagtgcttt gtagagagca aggtcttgcc tcgttcccta |
| 44041 | acccaatcct gggctttgtt tctttctttg atctatttct ctcttctgtt gttttctttc |
| 44101 | tttcaggaga cagggtcttg ctctgtcacc cagactggag tacagtgtct tgatactagc |
| 44161 | tcactgcaaa gtcaaattcc tgggctcaag ggatcctcct gcctcagcca cctgaggagc |
| 44221 | tggaactgca ggcctgcgac actgcaccca gctaattttt ttttcataaa tattatgctt |
| 44281 | ttgtacccag cttttttttt tttttttttt taactgcagc cttgacctcc caggcttaca |
| 44341 | tgatcctccc acctctgctt cctgagtagc tgtgattaca ggtgcatgcc accatgccca |
| 44401 | gtgaattaaa aaaaaaaaaa gtttgtagat atggggtccc actgtactgc ctaggctggt |
| 44461 | cttaaactcc tgagctcaag tgattctccc acctcagcct cctaaagtgc tgagattaca |
| 44521 | ggcataagcc cctggtgcct ggccccagct gaatttttgt tcttgtttct tcataaatat |
| 44581 | tctgtgtaag tacccagctg attgttttat tttttgtaga gatgggggtc ttgatatgtt |
| 44641 | gctcaagttg gtctcaaact actggcctca agcgatcttc ctgcctcagc ctcccaaagg |
| 44701 | gctgggattc caagcatgag ccaccacacc tgccacctct tctgttattt tctctccatc |
| 44761 | tggcattctc tgactctttc atctctacca tgatttgggc tttctcctct cccttctctt |
| 44821 | atttcttccc attctcctat ccccatatcc tccctgctaa ctcctgatac ccacagggcc |
| 44881 | cctcaatccc attttagtca gcttaagtaa caatagctac taaaacaaaa cccctaagaa |
| 44941 | tatggggtct taacacaaca gacttgtatt tctcactcat gtaaagtcca gttggcatgg |
| 45001 | ggggtaagga agggtccctc tgctccatgt agtctctcag ggatccaagc accttccatc |
| 45061 | ctgtggctct gcaatcctta ggatcttctg tagttctctg caggattcat tcattctaga |
| 45121 | tggaaataag attgtgcatg ggttgttttt atgggcatag atagcaatct gttcagccac |
| 45181 | ctggccacac ctaattgaaa gaggagctga gaaaggtagt ctcactgtga gtctaggaag |
| 45241 | aaaagtaaat ggatttgctg aattgctcat tcatctttgc cacttcctcc ttgatccttc |
| 45301 | agtttctcca ccactgcctc agctcccaag acaatgctgg actccctccc acatcacccc |
| 45361 | actgaccaag ctcctccttc cccctcaggt ctcacactct gaggatgact gtctagcttt |
| 45421 | caaagttcac caatacttta atgtagagct tatccagcct ggagcagtca aggtctacgc |
| 45481 | ctattacaac ctgggtgagc agccaaccta gggcctgggg tctgatggtt ccaggggcct |
| 45541 | gagagtccca ggtatatatg aattgtgggg atctgagaat gaaggtctaa ggagtccagg |
| 45601 | gatttgagca ttcgtagtat gaaggtccca cgggtctgag ggtcccaagg atctatgagt |
| 45661 | tgaggttctg aggttctgag gggatctgag aatgatggtc taagcaggcc agggatttca |
| 45721 | ggattagtaa tctgaaggtc ccagggtctg agagtcccaa ggatctatga gttggttcta |
| 45781 | gggatctgag acttgggggt ctgatgggtt caggggtctc agggtcttag gaatatgtga |
| 45841 | gttgcagggg gttctgaaaa taagggtcta aggattctag atatatgagg gttggaggcc |
| 45901 | tgcgtgtccc aggaatctat gaatttgggg tctgagggtc ccaggcttct gtgagttgag |
| 45961 | agtctaagag actcaagggt ctgagaatcc caaagatcag aaagtagagg gggtcttggg |
| 46021 | gtctgaggga tctgaggggt tgaagaccta gcatctccag gtctgaagac tgagaactgg |
| 46081 | ggatctgggc ctcccaggca tggtctttgg agggaggccc ttatcctctc atcttcacat |
| 46141 | cacatctgcc cgcagaggaa agctgtaccc ggttctacca tccggaaaag gaggatggaa |
| 46201 | agctgaacaa gctctgccgt gatgaactgt gccgctgtgc tgagggtgag ttccctggag |
| 46261 | ccgggaacag gtgggtctga gcaagccaca cttacccagg tcatctatcc catggtcagg |
| 46321 | gacccccaga cccataccca ggggatacca aggggggtag gctcccaggg ctggccacac |
| 46381 | ccatgggcag taggccccag ataaggagtg ggacttagac cctgtctcca ccccaccctg |
| 46441 | cagagaattg cttcatacaa aagtcggatg acaaggtcac cctggaagaa cggctggaca |
| 46501 | aggcctgtga gccaggagtg gactatggtg agtgggtgat gggtgggggt cacgcatgtt |
| 46561 | tagctgtgtg tgtccaattg tgtggtgggt ggtaggtgtg gttgtcatgg tgtggcttca |
| 46621 | ggctgtgggt gtgggtgact gtggtgtgtg tgagagcatg tattgtgagg ggccatgatt |
| 46681 | gtgtggggaa ccatgactgt gagtggccta ggtatgctca tgtgagaaaa ggtagatgtg |
| 46741 | gttgtatgca tcattgcgtg ggtggctgtg aggttgtagt tgtgtgtggc tgtggttgtg |
| 46801 | tgaggctgtg tggttgtaga tggcagtgag tgtgaggtcc tgaagttacg tatatgactg |
| 46861 | tagttttccg tggctatggt tgtgtgcatg gccatgaggc tacagtattt tgtgcatatg |
| 46921 | agtcactctc attgcatagt atgaatagta tgttactaga cattgtgggt ggctgtgacc |
| 46981 | tctgtgcatg cctatgagca cgactgtgtg tggatggtga catgggaccc tctatggttg |
| 47041 | tgtgtgtaat gaggggtggg ccatagtgtg actggctgtg attctgcaac tttctgcttg |
| 47101 | ggagagagag ccacatgccc gggtgcactt gcaaaccagg gtgcccctca tggtcaacct |
| 47161 | agcccaccac ccaaactgtc tgcctctccc ccacagtgta caagacccga ctggtcaagg |
| 47221 | ttcagctgtc caatgacttt gacgagtaca tcatggccat tgagcagacc atcaagtcag |
| 47281 | gtcaggctca gcacgctgcc tcccgtggct cttccctggc ttcctcccca cgactcagct |
| 47341 | tcttccctct cccctccact ccaggctcgg atgaggtgca ggttggacag cagcgcacgt |
| 47401 | tcatcagccc catcaagtgc agagaagccc tgaagctgga ggagaagaaa cactacctca |
| 47461 | tgtggggtct ctcctccgat ttctggggag agaagcccaa gtgagtgctt tccctgcgcg |
| 47521 | tgcgcgcgac cgcccgactg ccccgcccat gccacgccca caccattgtc acgcccctgc |
| 47581 | gccacgccca caccacgccc cttcctgacc tgccattctt ccctccagcc tcagctacat |
| 47641 | catcgggaag gacacttggg tggagcactg gcccgaggag gacgaatgcc aagacgaaga |
| 47701 | gaaccagaaa caatgccagg acctcggcgc cttcaccgag agcatggttg tctttgggtg |
| 47761 | ccccaactga ccacaccccc attcccccac tccagataaa gcttcagtta tatctcacgt |
| 47821 | gtctggagtt ctttgccaag agggagaggc tgaaatcccc agccgcctca cctgcagctc |
| 47881 | agctccatcc tacttgaaac ctcacctgtt cccaccgcat tttctcctgg cgttcgcctg |
| 47941 | ctagtgtgct gacttcttta gccaaggagc atggacctgc ctcacctgca cgtggcatgc |
| 48001 | acctgcgcct cacctccatt tcacctgcac actcaccggc agctcacagc cccttcacct |
| 48061 | cttcacttac cggcatcctc acctgttaat cttaccaatt tttttttatt ttattattat |
| 48121 | tactatttta agttccgggg tacatgtgca ggatgtgcag gtttgttaca taggtcaagt |
| 48181 | gtgccatggt ggtttcctgc acctatcaac ccatcaccta ggttttttgt ttgtgtgttt |
| 48241 | tgaggcagag tcttgttctg tcgcccaggc tggagtgcag tggcacaatc tcggctcact |
| 48301 | gcaacctcca cctcccgggt tcaagtgatt ctcctgcctt agcctcctga gtaggtggga |
| 48361 | ttacaggcgc ccgccacctt gcctgggtaa tttttgtatt tttggtagag acggggtttc |
| 48421 | accatgttgg ccaggctggt cttgaactcc tgatctcaag cgatccgccc gccttggcct |
| 48481 | cccaaagtgc tgggattaca ggcgtgagcc atcacaccca gccccctatt acctagttat |
| 48541 | tacgtccagg atgcattagg tcttttccct aatgttctcc ctgctcccaa tgttaccaat |
| 48601 | attttcatct gaatctttac ctgctcactc ctctgcaccc tcagctgaat ccatgtatgg |
| 48661 | gtttttgttg ttgttgtttt gtttttgtgg gtttttctgt tttttttttt tttttttttt |
| 48721 | ttttgagatg gagtttcact cttgtcgccc aggctggagt gcaatggcgc gatctcggct |
| 48781 | cactgtgacc cctcctcctg ggttcaagcg attctcctgc ctcagcctcc cgagtagctg |
| 48841 | tggttacagg cacacggcca ccacacctgg ctaatttttg tatttttatt agagacgggg |
| 48901 | tttcaccatg tcggccagac cggtctcgaa ctcctgacct caggtgatct gcccgcctcg |
| 48961 | gcctcccaaa gtgctgggat tgcaggcgtg agcctccgtg ccccgccagg gttttttgtt |
| 49021 | tttgtttttt agcatcctca cctggcccca acacctacat ctctatctta agcttacctg |
| 49081 | tatctttacc ttaacagcat tgttacctat attctcacct ttttccacct acatcctctc |
| 49141 | cggtgagtgt attttctctg catcttcatc tgggtcctca cctgcatctt tacctgcatg |
| 49201 | cttttctagg tattttcttg ggttcttgcc cacattctca cctacattct cacctgcaga |
| 49261 | tttacctatc ttcttactgt aactgcccaa tgggttcacc ttgcccgctg cctagacaga |
| 49321 | accgatttat cagacggggg atgcagtgga gaaagagtaa ttcgtgcaga acaagctgtg |
| 49381 | caggagacca gagttttatt attattcaaa tcagtctcct cgagcatttg gggatcagcg |
| 49441 | gttttaaaga tagtttggtg ggccagacgc agtggctcat gcctgtaatc ccaacacttt |
| 49501 | gggaggccga ggcaggtgga tcacctgagg tcagcagttc gagaccagcc tggccaacat |
| 49561 | gatgaaaccc cgtctctact aaaaatacaa aaattagcca ggcgtggtga tgcacacctg |
| 49621 | tagtcccagc tacttgagag gctgaggcag gagaatcgct tgaacccggg aggtggaggt |
| 49681 | tgcagtgagc cgagattgcg ccactgcact ccagcctggg tgacagagcg agacttcatc |
| 49741 | tcaaaataat aataataata atagtttggc aggtagaggt ttgggaagtg aggagtgttg |
| 49801 | attggtgagg ttgaagt |

The human C3 gene has 41 exons, as shown in Table 1, below.

TABLE 1

| Exon # | Position in C3 genomic sequence of SEQ ID NO: 1 |
|---|---|
| 1 | 5001-5136 |
| 2 | 6249-6441 |
| 3 | 7240-7405 |
| 4 | 7488-7558 |
| 5 | 11206-11300 |
| 6 | 11404-11486 |
| 7 | 11570-11660 |
| 8 | 12143-12245 |
| 9 | 12337-12463 |
| 10 | 13029-13144 |
| 11 | 13246-13395 |
| 12 | 14456-14665 |
| 13 | 14807-15013 |
| 14 | 15810-15968 |
| 15 | 17723-17852 |
| 16 | 18115-18186 |
| 17 | 18379-18576 |
| 18 | 23073-23181 |
| 19 | 23440-23525 |
| 20 | 27858-28000 |
| 21 | 28096-28308 |
| 22 | 28993-29059 |
| 23 | 29187-29273 |
| 24 | 31018-31221 |
| 25 | 32165-32240 |
| 26 | 32569-32728 |
| 27 | 34925-35023 |
| 28 | 38750-38906 |
| 29 | 39365-39528 |
| 30 | 40506-40664 |
| 31 | 40818-40877 |
| 32 | 41002-41092 |
| 33 | 41213-41264 |
| 34 | 43423-43510 |
| 35 | 43622-43711 |
| 36 | 45389-45494 |
| 37 | 46156-46245 |
| 38 | 46444-46527 |
| 39 | 47197-47280 |
| 40 | 47365-47500 |
| 41 | 47629-47817 |

The amino acid sequence of human C3 is shown below:

(SEQ ID NO: 2)
MGPTSGPSLLLLLLTHLPLALGSPMYSIITPNILRLESEETMVLEAHDAQ
GDVPVTVTVHDFPGKKLVLSSEKTVLTPATNHMGNVTFTIPANREFKSEK
GRNKFVTVQATFGTQVVEKVVLVSLQSGYLFIQTDKTIYTPGSTVLYRIF
TVNHKLLPVGRTVMVNIENPEGIPVKQDSLSSQNQLGVLPLSWDIPELVN
MGQWKIRAYYENSPQQVFSTEFEVKEYVLPSFEVIVEPTEKFYYIYNEKG
LEVTITARFLYGKKVEGTAFVIFGIQDGEQRISLPESLKRIPIEDGSGEV
VLSRKVLLDGVQNPRAEDLVGKSLYVSATVILHSGSDMVQAERSGIPIVT
SPYQIHFTKTPKYFKPGMPFDLMVFVTNPDGSPAYRVPVAVQGEDTVQSL
TQGDGVAKLSINTHPSQKPLSITVRTKKQELSEAEQATRTMQALPYSTVG
NSNNYLHLSVLRTELRPGETLNVNFLLRMDRAHEAKIRYYTYLIMNKGRL
LKAGRQVREPGQDLVVLPLSITTDFIPSFRLVAYYTLIGASGQREVVADS
VWVDVKDSCVGSLVVKSGQSEDRQPVPGQQMTLKIEGDHGARVVLVAVDK
GVFVLNKKNKLTQSKIWDVVEKADIGCTPGSGKDYAGVFSDAGLIFTSSS
GQQTAQRAELQCPQPAARRRRSVQLTEKRMDKVGKYPKELRKCCEDGMRE
NPMRFSCQRRTRFISLGEACKKVELDCCNYITELRRQHARASHLGLARSN
LDEDIIAEENIVSRSEFPESWLWNVEDLKEPPKNGISTKLMNIFLKDSIT
TWEILAVSMSDKKGICVADPFEVTVMQDFFIDLRLPYSVVRNEQVEIRAV
LYNYRQNQELKVRVELLHNPAFCSLATTKRRHQQTVTIPPKSSLSVPYVI
VPLKTGLQEVEVKAAVYHHFISDGVRKSLKVVPEGIRMNKTVAVRILDPE
RLGREGVQKEDIPPADLSDQVPDIESETRILLQGTPVAQMTEDAVDAERL
KHLIVTPSGCGEQNMIGMTPTVIAVHYLDETEQWEKFGLEKRQGALELIK
KGYTQQLAFRQPSSAFAAFVKRAPSTWLTAYVVKVFSLAVNLIAIDSQVL
CGAVKWLILEKQKPDGVFQEDAPVIHQEMIGGLRNNNEKDMALTAFVLIS
LQEAKDICEEQVNSLPGSITKAGDFLEANYMNLQRSYTVAIAGYALAQMG
RLKGPLLNKFLTTAKDKNRWEDPGKQLYNVEATSYALLALLQLKDFDFVP
PVVRWLNEQRYYGGGYGSTQATFMVFQALAQYQKDAPDHQELNLDVSLQL
PSRSSKITHRIHWESASLLRSEETKENEGFTVTAEGKGQGTLSVVTMYHA
KAKDQLTCNKFDLKVTIKPAPETEKRPQDAKNTMILEICTRYRGDQDATM
SILDISMMTGFAPDTDDLKQLANGVDRYISKYELDKAFSDRNTLIIYLDK
VSHSEDDCLAFKVHQYFNVELIQPGAVKVYAYYNLEESCTRFYHPEKEDG
KLNKLCRDELCRCAEENCFIQKSDDKVTLEERLDKACEPGVDYVYKTRLV
KVQLSNDFDEYIMAIEQTIKSGSDEVQVGQQRTFISPIKCREALKLEEKK
HYLMWGLSSDFWGEKPNLSYIIGKDTWVEHWPEEDECQDEENQKQCQDLG
AFTESMVVFGCPN

In some embodiments, a target nucleic acid is a polynucleotide encoding a complement protein described herein, e.g., a C3-encoding polynucleotide. In some embodiments, a target nucleic acid is or comprises an exon (or a portion thereof) of a human C3 genomic sequence (e.g., of SEQ ID NO:1, e.g., an exon listed in Table 1). In some embodiments, a target nucleic acid is or comprises an intron (or a portion thereof) of a human C3 genomic sequence (e.g., of SEQ ID NO:1).

In some embodiments, a genomic edit comprises a deletion, substitution, and/or insertion of one or more nucleotides within an exon (or a portion thereof) of a human C3 genomic sequence (e.g., of SEQ ID NO:1, e.g., an exon listed in Table 1); and/or within an intron (or a portion thereof) of a human C3 genomic sequence (e.g., of SEQ ID NO:1).

In some embodiments, a genomic edit comprises a single base edit. In some embodiments, a single base edit reduces expression and/or function of a complement protein (e.g., C3), e.g., relative to wildtype complement protein (e.g., C3). In some embodiments, a single base edit introduces a premature stop codon in the C3 coding sequence that leads to a truncated and/or non-functional C3 protein, e.g., relative to wildtype C3 protein. In certain embodiments, the premature stop codon is TAG (Amber), TGA (Opal), or TAA (Ochre).

In some embodiments, a premature stop codon is generated from a CAG to TAG change on the coding strand via deamination of the C (using a base editor described herein and a gRNA that targets the appropriate genomic locus). In some embodiments, a premature stop codon is generated from a CGA to TGA change on the coding strand via deamination of the C (using a base editor described herein and a gRNA that targets the appropriate genomic locus). In some embodiments, a premature stop codon is generated from a CAA to TAA change on the coding strand via deamination of the C (using a base editor described herein and a gRNA that targets the appropriate genomic locus). Any “CAG”, “CGA”, and/or “CAA” codon within a target gene (e.g., a gene encoding a complement protein, e.g., C3) can be edited to a “TAG”, “TGA”, or “TAA”, respectively. Exemplary codons within the human C3 gene that can be edited to corresponding stop codons are listed in Table 2:

TABLE 2
Exemplary single-base edits to human C3 gene
(SEQ ID NO: 1) to introduce a stop codon

| | Edited base | Original | | | |
|---|---|---|---|---|---|
| Exon | position | codon in | | Corresponding | |
| (see | from exon | SEQ ID | Edited | AA of SEQ | AA |
| Table 1) | start | NO: 1 | codon | ID NO: 2 | change |
| 2 | 74 | CAA | TAG | Gln50 | Q → Stop |
| 3 | 58 | CAG | TAG | Gln109 | Q → Stop |
| 3 | 76 | CAA | TAG | Gln115 | Q → Stop |
| 3 | 209 | CAG | TAG | Gln126 | Q → Stop |
| 3 | 230 | CAG | TAG | Gln133 | Q → Stop |
| 5 | 25 | CAG | TAG | Gln177 | Q → Stop |
| 5 | 43 | CAG | TAG | Gln183 | Q → Stop |
| 5 | 49 | CAG | TAG | Gln185 | Q → Stop |
| 6 | 8 | CAG | TAG | Gln203 | Q → Stop |
| 6 | 20 | CGA | TGA | Arg207 | R → Stop |
| 6 | 44 | CAG | TAG | Gln215 | Q → Stop |
| 6 | 47 | CAG | TAG | Gln216 | Q → Stop |
| 8 | 53 | CAG | TAG | Gln276 | Q → Stop |
| 8 | 65 | CAG | TAG | Gln280 | Q → Stop |
| 9 | 58 | CAG | TAG | Gln312 | Q → Stop |
| 9 | 67 | CGA | TGA | Arg315 | R → Stop |
| 10 | 15 | CAG | TAG | Gln340 | Q → Stop |
| 10 | 57 | CAG | TAG | Gln354 | Q → Stop |
| 11 | 37 | CGA | TGA | Arg386 | R → Stop |
| 11 | 55 | CAG | TAG | Gln391 | Q → Stop |
| 11 | 73 | CAG | TAG | Gln398 | Q → Stop |
| 11 | 85 | CAG | TAG | Gln402 | Q → Stop |
| 11 | 130 | CAG | TAG | Gln417 | Q → Stop |
| 12 | 16 | CAG | TAG | Gln429 | Q → Stop |
| 12 | 37 | CAG | TAG | Gln436 | Q → Stop |
| 12 | 55 | CAG | TAG | Gln442 | Q → Stop |
| 12 | 172 | CGA | TGA | Arg478 | R → Stop |
| 13 | 43 | CGA | TGA | Arg508 | R → Stop |
| 13 | 55 | CAG | TAG | Gln512 | Q → Stop |
| 13 | 148 | CAG | TAG | Gln543 | Q → Stop |
| 14 | 19 | CAG | TAG | Gln569 | Q → Stop |
| 14 | 34 | CAG | TAG | Gln574 | Q → Stop |
| 14 | 49 | CAG | TAG | Gln579 | Q → Stop |
| 14 | 52 | CAG | TAG | Gln580 | Q → Stop |
| 14 | 151 | CAG | TAG | Gln613 | Q → Stop |
| 15 | 109 | CAG | TAG | Gln652 | Q → Stop |
| 15 | 112 | CAG | TAG | Gln653 | Q → Stop |
| 16 | 6 | CAG | TAG | Gln661 | Q → Stop |
| 16 | 15 | CAG | TAG | Gln664 | Q → Stop |
| 16 | 30 | CGA | TGA | Arg669 | R → Stop |
| 16 | 45 | CAG | TAG | Gln674 | Q → Stop |
| 16 | 60 | CGA | TGA | Arg679 | R → Stop |
| 17 | 162 | CAG | TAG | Gln747 | Q → Stop |
| 18 | 45 | CGA | TGA | Arg764 | R → Stop |
| 20 | 81 | CGA | TGA | Arg841 | R → Stop |
| 20 | 90 | CAG | TAG | Gln844 | Q → Stop |
| 20 | 102 | CGA | TGA | Arg848 | R → Stop |
| 20 | 126 | CAG | TAG | Gln856 | Q → Stop |
| 20 | 132 | CAG | TAG | Gln858 | Q → Stop |
| 21 | 64 | CAG | TAG | Gln883 | Q → Stop |
| 21 | 67 | CAG | TAG | Gln884 | Q → Stop |
| 21 | 139 | CAG | TAG | Gln908 | Q → Stop |
| 23 | 9 | CAG | TAG | Gln958 | Q → Stop |
| 23 | 45 | CAG | TAG | Gln970 | Q → Stop |
| 23 | 84 | CAG | TAG | Gln983 | Q → Stop |
| 24 | 15 | CAG | TAG | Gln989 | Q → Stop |
| 24 | 87 | CAG | TAG | Gln1013 | Q → Stop |
| 24 | 147 | CAG | TAG | Gln1033 | Q → Stop |
| 24 | 177 | CAG | TAG | Gln1043 | Q → Stop |
| 25 | 9 | CAG | TAG | Gln1055 | Q → Stop |
| 25 | 12 | CAG | TAG | Gln1056 | Q → Stop |
| 25 | 27 | CAA | TAA | Gln1061 | Q → Stop |
| 26 | 62 | CAA | TAA | Gln1098 | Q → Stop |
| 26 | 104 | CAG | TAG | Gln1122 | Q → Stop |
| 26 | 125 | CAG | TAG | Gln1129 | Q → Stop |
| 26 | 148 | CAA | TAA | Gln1137 | Q → Stop |
| 27 | 64 | CAG | TAG | Gln1152 | Q → Stop |
| 27 | 91 | CAG | TAG | Gln1161 | Q → Stop |
| 28 | 61 | CAG | TAG | Gln1184 | Q → Stop |
| 28 | 103 | CAG | TAG | Gln1198 | Q → Stop |
| 29 | 30 | CAG | TAG | Gln1226 | Q → Stop |
| 29 | 78 | CAG | TAG | Gln1242 | Q → Stop |
| 29 | 129 | CAG | TAG | Gln1259 | Q → Stop |
| 29 | 162 | CAG | TAG | Gln1270 | Q → Stop |
| 30 | 19 | CAA | TAA | Gln1277 | Q → Stop |
| 30 | 31 | CAA | TAA | Gln1280 | Q → Stop |
| 30 | 37 | CAA | TAA | Gln1282 | Q → Stop |
| 30 | 58 | CAG | TAG | Gln908 | Q → Stop |
| 30 | 85 | CAA | TAA | Gln1299 | Q → Stop |
| 30 | 148 | CGA | TGA | Arg1320 | R → Stop |
| 31 | 46 | CAA | TAA | Gln1339 | Q → Stop |
| 32 | 34 | CAA | TAA | Gln1355 | Q → Stop |
| 33 | 12 | CAG | TAG | Gln1378 | Q → Stop |
| 34 | 14 | CAG | TAG | Gln1396 | Q → Stop |
| 34 | 86 | CAG | TAG | Gln1420 | Q → Stop |
| 36 | 43 | CAA | TAA | Gln1465 | Q → Stop |
| 36 | 67 | CAG | TAG | Gln1473 | Q → Stop |
| 38 | 15 | CAA | TAA | Gln1521 | Q → Stop |
| 39 | 12 | CGA | TGA | Arg1548 | R → Stop |
| 39 | 27 | CAG | TAG | Gln1553 | Q → Stop |
| 39 | 69 | CAG | TAG | Gln1567 | Q → Stop |
| 40 | 15 | CAG | TAG | Gln1577 | Q → Stop |
| 40 | 24 | CAG | TAG | Gln1580 | Q → Stop |
| 40 | 27 | CAG | TAG | Gln1581 | Q → Stop |
| 41 | 62 | CAA | TAA | Gln1638 | Q → Stop |
| 41 | 77 | CAG | TAG | Gln1643 | Q → Stop |
| 41 | 83 | CAA | TAA | Gln1645 | Q → Stop |
| 41 | 89 | CAG | TAG | Gln1647 | Q → Stop |

In some embodiments, a genomic edit comprises an edit of a human C3 gene that leads to expression of a mutant C3 protein that has reduced and/or no ability to be cleaved by C3 convertase. In some embodiments, such mutant C3 protein is a competitive inhibitor of a C3 convertase (e.g., mutant C3 protein binds C3 convertase, but is not cleaved by C3 convertase). Such an edit can be made by targeting nucleic acids encoding a region within and/or proximate to the putative cleavage site of C3. In some embodiments, a genomic edit comprises a deletion, substitution, and/or insertion of one or more nucleotides of a codon encoding one or more of amino acids 662 to 681 of SEQ ID NO:2 (e.g., one or more of amino acids 665 to 671 of SEQ ID NO:2). In some embodiments, a genomic edit deletes all or a portion of a codon encoding one or more of amino acids 662 to 681 of SEQ ID NO:2 (e.g., one or more of amino acids 665 to 671 of SEQ ID NO:2). In some embodiments, a genomic edit comprises a single base edit of a codon encoding one or more of amino acids 662 to 681 of SEQ ID NO:2 (e.g., one or more of amino acids 665 to 671 of SEQ ID NO:2), such that the edited codon encodes an amino acid that is different from the original amino acid. In some embodiments, such single base edit is produced using a base editor described herein and a gRNA that targets the appropriate genomic locus. Exemplary single-base edits to remove and/or abrogate a cleavage site are listed in Table 3.

TABLE 3
Exemplary single-base edits to the C3 gene to remove cleavage site

| | Edited base | Original | | | |
|---|---|---|---|---|---|
| Exon | position | codon in | | Corresponding | |
| (See | from exon | SEQ ID | Edited | AA of SEQ | AA |
| Table 1) | start | NO: 1 | codon | ID NO: 2 | change |
| 16 | 18 | CCA | TCA | Pro665 | P → S |
| 16 | 19 | CCA | CTA | Pro665 | P → L |
| 16 | 21 | GCC | ACC | Ala666 | A → T |
| 16 | 22 | GCC | GTC | Ala666 | A → V |
| 16 | 24 | GCC | ACC | Ala667 | A → T |
| 16 | 25 | GCC | GTC | Ala667 | A → V |
| 16 | 27 | CGC | TGC | Arg668 | R → C |
| 16 | 28 | CGC | CAC | Arg668 | R → H |
| 16 | 30 | CGA | TGA | Arg669 | R → Stop |
| 16 | 31 | CGA | CAA | Arg669 | R → Q |
| 16 | 33 | CGC | TGC | Arg670 | R → C |
| 16 | 34 | CGC | CAC | Arg670 | R → H |
| 16 | 36 | CGT | TGT | Arg671 | R → C |
| 16 | 37 | CGT | CAT | Arg671 | R → H |

In some embodiments, a genomic edit comprises an edit of a human C3 gene that leads to expression of C3 protein that has mutation within a thioester domain (see, e.g., Isaac et al., JBC 267:10062-10069 (1992). In some embodiments, such mutation leads to reduced function of the thioester domain, relative to wild type C3. Such an edit can be made by targeting nucleic acids encoding a region within a thioester domain. In some embodiments, a genomic edit comprises a deletion, substitution, and/or insertion of one or more nucleotides of one or more of exons 24-30 of SEQ ID NO:1 (see Table 1). In some embodiments, a genomic edit comprises a deletion, substitution, and/or insertion of one or more nucleotides of exon 24 of SEQ ID NO:1 (see Table 1). In some embodiments, a genomic edit comprises a deletion, substitution, and/or insertion of all or a portion of a codon encoding one or more of amino acids 1005 to 1021 of SEQ ID NO:2. In some embodiments, a genomic edit comprises a single base edit of a codon encoding one or more of amino acids 1005 to 1021 of SEQ ID NO:2, such that the edited codon encodes an amino acid that is different from the original amino acid. In some embodiments, such single base edit is produced using a base editor described herein and a gRNA that targets the appropriate genomic locus. Exemplary single-base edits to codons encoding thioester domain amino acids are listed in Table 4.

TABLE 4
Exemplary single-base edits within
C3 gene encoding thioester domain

| | Edited base | Original | | | |
|---|---|---|---|---|---|
| Exon | position | codon in | | Corresponding | |
| (See | from exon | SEQ ID | Edited | AA of SEQ | AA |
| Table 1) | start | NO: 1 | codon | ID NO: 2 | change |
| 24 | 69 | CCC | TCC | Pro1007 | P → S |
| 24 | 70 | CCC | CTC | Pro1007 | P → L |
| 24 | 78 | TGC | CGC | Cys1010 | C → R |
| 24 | 79 | TGC | TAC | Cys1010 | C → Y |
| 24 | 84 | GAA | AAA | Glu1012 | E → K |
| 24 | 85 | GAA | GGA | Glu1012 | E → G |
| 24 | 87 | CAG | TAG | Gln1013 | Q → Stop |
| 24 | 88 | CAG | CGG | Gln1013 | Q → R |
| 24 | 93 | ATG | GTG | Met1015 | M → V |
| 24 | 94 | ATG | ACG | Met1015 | M → T |
| 24 | 95 | ATG | ATA | Met1015 | M → I |
| 24 | 108 | CCC | TCC | Pro1020 | P → S |
| 24 | 109 | CCC | CTC | Pro1020 | P → L |

Two major polymorphic allotypes of C3 are known: C3S (with frequencies of 0.79 and 0.99 in white and Asian populations, respectively) and C3F (see, e.g., Rodriguez et al., JBC 290:2334-2350 (2015)). C3F is associated with diseases, including IgA nephropathy, systemic vasculitis, partial lipodystrophy, membranoproliferative glomerulonephritis type II, and age-related macular degeneration. C3S includes an Arg at position 102, as depicted in SEQ ID NO:2, whereas C3F includes a Gly (instead of an Arg) at position 102 of SEQ ID NO:2. Presence of Arg at position 102 allows formation of an activity-regulating salt bridge (see Rodriguez et al., JBC 290:2334-2350 (2015)).

In some embodiments, a genomic edit comprises an edit of a human C3F-expressing gene that leads to expression of human C3S protein. Such an edit can be made by targeting a codon encoding a Gly at position 102 of SEQ ID NO:2, for example, as shown in Table 5.

TABLE 5
Exemplary edits to the C3 codon encoding Gly at position 102

| | Edited base | Original | | | |
|---|---|---|---|---|---|
| Exon | position | codon in | | Corresponding | Amino |
| (See | from exon | SEQ ID | Edited | AA of SEQ | acid |
| Table 1) | start | NO: 1 | codon | ID NO: 2 | change |
| 3 | 37 | GGC | CGC | Gly102 | G → R |

### Complement-Mediated Disorders and Diseases

In some embodiments, a gene therapy described herein (e.g., a genome editing system described herein) is administered to a subject suffering from or at risk of complement-mediated damage to an organ, tissue, or cells. In some embodiments, a gene therapy described herein is administered in combination with one or more additional complement inhibitors to a subject suffering from or at risk of complement-mediated damage to an organ, tissue, or cells. In some embodiments, a gene therapy described herein is contacted with an organ, tissue, or cells ex vivo. The organ, tissue, or cells can be introduced into a subject and can be protected from damage that would otherwise be caused by the recipient's complement system.

Certain uses of interest include: (1) protecting red blood cells (RBCs) from complement-mediated damage in individuals with disorders such as paroxysmal nocturnal hemoglobinuria or atypical hemolytic uremic syndrome or other disorders characterized by complement-mediated RBC lysis; (2) protecting transplanted organs, tissues, and cells from complement-mediated damage; (3) reducing ischemia/reperfusion (I/R) injury (e.g., in individuals suffering from trauma, vascular obstruction, myocardial infarction, or other situations in which I/R injury may occur); and (4) protecting various body structures (e.g., the retina) or membranes (e.g., synovial membrane) that may be exposed to complement components from complement mediated damage in any of a variety of different complement-mediated disorders. The beneficial effects of inhibiting complement activation at the surface of cells or other body structures are not limited to those resulting directly from protection of the cells or structures themselves against direct complement-mediated damage (e.g., preventing cell lysis). For example, inhibiting complement activation may reduce the generation of anaphylotoxins and resulting influx/activation of neutrophils and other pro-inflammatory events and/or reduce potentially damaging release of intracellular contents, thereby potentially having beneficial effects on remote organ systems or throughout the body.

A. Blood Cell Protection

In some embodiments, a gene therapy described herein, alone or in combination with one or more additional complement inhibitors, is used to protect blood cells against complement-mediated damage. The blood cells may be any cellular component of the blood, e.g., red blood cells (RBCs), white blood cells (WBCs), and/or platelets. A variety of disorders are associated with complement-mediated damage to blood cells. Such disorders can result, for example, from deficiencies or defects in one or more of an individual's cellular or soluble CRPs, e.g., due to (a) mutation(s) in the gene(s) encoding such proteins; (b) mutation(s) in genes required for production or proper function of one or more CRPs, and/or (c) presence of autoantibodies to one or more CRPs. Complement-mediated RBC lysis can result from the presence of autoantibodies against RBC antigens which may arise due to a diverse set of causes (often being idiopathic). Individuals having such mutation(s) in genes encoding CRPs and/or having antibodies against CRPs or against their own RBCs are at increased risk of disorders involving complement-mediated RBC damage. Individuals who have had one or more episodes characteristic of a disorder are at increased risk of a recurrence.

Paroxysmal nocturnal hemoglobinuria (PNH) is a relatively rare disorder comprising an acquired hemolytic anemia characterized by complement-mediated intravascular hemolysis, hemoglobinuria, bone marrow failure, and thrombophilia (propensity to develop blood clots). It affects an estimated 16 individuals per million worldwide, occurs in both sexes, and can arise at any age, frequently striking young adults (Bessler, M. & Hiken, J., Hematology Am Soc Hematol Educ Program, 104-110 (2008); Hillmen, P. Hematology Am Soc Hematol Educ Program, 116-123 (2008)). PNH is a chronic and debilitating disease punctuated by acute hemolytic episodes and results in significant morbidities and reduced life expectancy. In addition to anemia, many patients experience abdominal pain, dysphagia, erectile dysfunction, and pulmonary hypertension, and are at increased risk of renal failure and thromboembolic events.

PNH was first described as a distinct entity in the 1800s, but it was only in the 1950s, with discovery of the alternative pathway of complement activation, that the cause of hemolysis in PNH was firmly established (Parker C J. Paroxysmal nocturnal hemoglobinuria: an historical overview. Hematology Am Soc Hematol Educ Program. 93-103 (2008)). CD55 and CD59 are normally attached to the cell membrane via glycosyl phosphatidylinositol (GPI) anchors (glycolipid structures that anchor certain proteins to the plasma membrane). PNH arises as a consequence of nonmalignant clonal expansion of hematopoietic stem cell(s) that have acquired a somatic mutation in the PIGA gene, which encodes a protein involved in synthesis of GPI anchors (Takeda J, et al. Deficiency of the GPI anchor caused by a somatic mutation of the PIG-A gene in paroxysmal nocturnal hemoglobinuria. Cell. 73:703-711 (1993)). Progeny of such stem cells are deficient in GPI-anchored proteins, including CD55 and CD59. This defect renders these cells susceptible to complement-mediated RBC lysis. Flow cytometric analysis using antibodies to GPI-anchored proteins is often used for diagnosis. It detects deficiency of GPI-anchored proteins at the cell surface and allows determination of the degree of deficiency and the proportion of affected cells (Brodsky R A. Advances in the diagnosis and therapy of paroxysmal nocturnal hemoglobinuria. Blood Rev. 22(2):65-74 (2008). PNH type III RBCs are completely deficient in GPI-linked proteins and are highly sensitive to complement whereas PNH type II RBCs have a partial deficiency and are less sensitive. FLAER is a fluorescently labeled inactive variant of proaerolysin (a bacterial toxin that binds GPI anchors) and is increasingly used together with flow cytometry for diagnosis of PNH. Lack of binding of FLAER to granulocytes is sufficient for diagnosis of PNH. In some embodiments, a gene therapy described herein, alone or in combination with one or more additional complement inhibitors, protects PNH RBCs, from deposition of C3b. In some embodiments a gene therapy described herein, alone or in combination with one or more additional complement inhibitors, inhibits intravascular and extravascular hemolysis in a subject suffering from PNH.

In some embodiments, a gene therapy described herein, alone or in combination with one or more additional complement inhibitors, is administered to a subject suffering from atypical hemolytic syndrome (aHUS). aHUS is a chronic disorder characterized by microangiopathic hemolytic anemia, thrombocytopenia, and acute renal failure and is caused by inappropriate complement activation, often due to mutations in genes encoding complement regulatory proteins (Warwicker, P., et al.. Kidney Int 53, 836-844 (1998); Kavanagh, D. & Goodship, T. Pediatr Nephrol 25, 2431-2442 (2010). Mutations in the complement factor H (CFH) gene are the most common genetic abnormality in patients with aHUS, and 60-70% of these patients die or reach end stage renal failure within one year after disease onset (Kavanagh & Goodship, supra.) Mutations in factor I, factor B, C3, factor H-related proteins 1-5, and thrombomodulin have also been described. Other causes of aHUS include autoantibodies against complement regulatory proteins such as CFH. In some embodiments, a gene therapy described herein, alone or in combination with one or more additional complement inhibitors, is administered to a subject that has been identified as having a mutation in factor I, factor B, C3, factor H-related proteins 1-5, or thrombomodulin or has been identified as having antibodies against a complement regulatory protein, e.g., CFH.

Complement-mediated hemolysis occurs in a diverse group of other conditions including autoimmune hemolytic anemias that involve antibodies that bind to RBCs and lead to complement-mediated hemolysis. For example, such hemolysis can occur in primary chronic cold agglutinin disease and certain reactions to drugs and other foreign substances (Berentsen, S., et al., Hematology 12, 361-370 (2007); Rosse, W. F., Hillmen, P. & Schreiber, A. D. Hematology Am Soc Hematol Educ Program, 48-62 (2004)). In some embodiments, a gene therapy described herein, alone or in combination with one or more additional complement inhibitors, is administered to a subject suffering from or at risk of chronic cold agglutinin disease. In another embodiment, a gene therapy described herein, alone or in combination with one or more additional complement inhibitors, is used to treat a subject suffering from or at risk of the HELLP syndrome, which is defined by the existence of hemolysis, elevated liver enzymes, and low platelet count and is associated with mutations in complement regulatory protein(s) in at least some subjects (Fakhouri, F., et al., 112: 4542-4545 (2008)).

In some embodiments, a gene therapy described herein, alone or in combination with one or more additional complement inhibitors, is administered to a subject suffering from or at risk of warm autoimmune hemolytic anemia.

In other embodiments, a gene therapy described herein, alone or in combination with one or more additional complement inhibitors, is used to protect RBCs or other cellular components of blood to be transfused into a subject. Certain examples of such uses are discussed further in below.

B. Transplantation

Transplantation is a therapeutic approach of increasing importance, providing a means to replace organs and tissues that have been damaged through trauma, disease, or other conditions. Kidneys, liver, lungs, pancreas, and heart are among the organs that can be successfully transplanted. Tissues that are frequently transplanted include bones, cartilage, tendons, cornea, skin, heart valves, and blood vessels. Pancreatic islet or islet cell transplantation is a promising approach for treatment of diabetes, e.g., type I diabetes. For purposes of the invention, an organ, tissue, or cell (or population of cells) that is be transplanted, is being transplanted, or has been transplanted may be referred to as a “graft”. For purposes hereof, a blood transfusion is considered a “graft”.

Transplantation subjects the graft to a variety of damaging events and stimuli that can contribute to graft dysfunction and, potentially, failure. For example, ischemia-reperfusion (I/R) injury is a common and significant cause of morbidity and mortality in the case of many grafts (particularly solid organs) and can be a major determinant of likelihood of graft survival. Transplant rejection is one of the major risks associated with transplants between genetically different individuals and can lead to graft failure and a need to remove the graft from the recipient.

A graft can be contacted with a gene therapy described herein, alone or in combination with one or more additional complement inhibitors, that inhibits C3 expression prior to, during, and/or after being transplanted, in various embodiments of the disclosure. In another embodiment, a gene therapy described herein, alone or in combination with one or more additional complement inhibitors, is administered to a donor prior to removal of the graft. In some embodiments, a gene therapy described herein, alone or in combination with one or more additional complement inhibitors, is administered to a recipient during and/or after the introduction of the graft. In some embodiments, a gene therapy described herein, alone or in combination with one or more additional complement inhibitors, is delivered locally to the transplanted graft. In some embodiments, a gene therapy described herein, alone or in combination with one or more additional complement inhibitors, is administered to a recipient prior to the introduction of the graft. In some embodiments the subject receives one or more additional doses of the gene therapy, and/or one or more additional complement inhibitors after receiving the graft.

In some embodiments, a graft is or comprises a solid organ such as a kidney, liver, lung, pancreas, or heart. In some embodiments, a graft is or comprises bone, cartilage, fascia, tendon, ligament, cornea, sclera, pericardium, skin, heart valve, blood vessel, amniotic membrane, or dura mater. In some embodiments, a graft comprises multiple organs such as a heart-lung or pancreas-kidney graft. In some embodiments, a graft comprises less than a complete organ or tissue. For example, a graft may contain a portion of an organ or tissue, e.g., a liver lobe, section of blood vessel, skin flap, or heart valve. In some embodiments, a graft comprises a preparation comprising isolated cells or tissue fragments that have been isolated from their tissue of origin but retain at least some tissue architecture, e.g., pancreatic islets. In some embodiments, a preparation comprises isolated cells that are not attached to each other via connective tissue, e.g., hematopoietic stem cells or progenitor cells derived from peripheral and/or cord blood, or whole blood or any cell-containing blood product such as red blood cells (RBCs) or platelets. In some embodiments a graft is obtained from a deceased donor (e.g., a “donation after brain death” (DBD) donor or “donation after cardiac death” donor). In some embodiments, depending on the particular type of graft, a graft is obtained from a living donor. For example, kidneys, liver sections, blood cells, are among the types of grafts that can often be obtained from a living donor without undue risk to the donor and consistent with sound medical practice.

In some embodiments, a graft is a xenograft (i.e., the donor and recipient are of different species). In some embodiments a graft is an autograft (i.e., a graft from one part of the body to another part of the body in the same individual). In some embodiments, a graft is an isograft (i.e., the donor and recipient are genetically identical). In most embodiments, the graft is an allograft (i.e., the donor and recipient are genetically non-identical members of the same species). In the case of an allograft, the donor and recipient may or may not be genetically related (e.g., family members). Typically, the donor and recipient have compatible blood groups (at least ABO compatibility and optionally Rh, Kell and/or other blood cell antigen compatibility). The recipient's blood may have been screened for alloantibodies to the graft and/or the recipient and donor since the presence of such antibodies can lead to hyperacute rejection (i.e., rejection beginning almost immediately, e.g., within several minutes after the graft comes into contact with the recipient's blood). A complement-dependent cytotoxicity (CDC) assay can be used to screen a subject's serum for anti-HLA antibodies. The serum is incubated with a panel of lymphocytes of known HLA phenotype. If the serum contains antibodies against HLA molecules on the target cells, cell death due to complement-mediated lysis occurs. Using a selected panel of target cells allows one to assign specificity to the detected antibody. Other techniques useful for determining the presence or absence anti-HLA antibodies and, optionally, determining their HLA specificity, include ELISA assays, flow cytometry assays, microbead array technology (e.g., Luminex technology). The methodology for performing these assays is well known, and a variety of kits for performing them are commercially available.

In some embodiments a gene therapy described herein, alone or in combination with one or more additional complement inhibitors, inhibits complement-mediated rejection. For example, in some embodiments, a gene therapy described herein, alone or in combination with one or more additional complement inhibitors, inhibits hyperacute rejection. Hyperacute rejection is caused at least in part by antibody-mediated activation of the recipient's complement system via the classical pathway and resulting MAC deposition on the graft. It typically results from the presence in the recipient of pre-existing antibodies that react with the graft. While it is desirable to attempt to avoid hyperacute rejection by appropriate matching prior to transplantation, it may not always possible to do so due, e.g., to time and/or resource constraints. Furthermore, some recipients (e.g., multiply transfused individuals, individuals who have previously received transplants, women who have had multiple pregnancies) may already have so many pre-formed antibodies, potentially including antibodies to antigens that are not typically tested for, that it can be difficult or perhaps almost impossible to obtain with confidence a compatible graft in a timely manner. Such individuals are at increased risk of hyperacute rejection.

In some embodiments, a gene therapy described herein, alone or in combination with one or more additional complement inhibitors, inhibits acute rejection or graft failure. As used herein, “acute rejection” refers to rejection occurring between at least 24 hours, typically at least several days to a week, after a transplant, up to 6 months after the transplant. Acute antibody-mediated rejection (AMR) often involves an acute rise in donor-specific alloantibody (DSA) in the first few weeks after transplantation. Without wishing to be bound by any theory, it is possible that pre-existing plasma cells and/or the conversion of memory B cells to new plasma cells play a role in the increased DSA production. Such antibodies can result in complement-mediated damage to the graft. Without wishing to be bound by any theory, inhibiting complement activation at the graft may reduce leukocyte (e.g., neutrophil) infiltration, another contributor to acute graft failure.

In some embodiments, a gene therapy described herein, alone or in combination with one or more additional complement inhibitors, inhibits complement-mediated I/R injury to a graft. As discussed further below, I/R injury can occur upon reperfusion of tissue whose blood supply has been temporarily disrupted, as occurs in transplanted organs. Reducing I/R injury would reduce the likelihood of acute graft dysfunction or reduce its severity, and reduce the likelihood of acute graft failure.

In some embodiments, a gene therapy described herein, alone or in combination with one or more additional complement inhibitors, inhibits chronic rejection and/or chronic graft failure. As used herein, “chronic rejection or graft failure” refers to rejection or failure occurring at least 6 months post-transplant, e.g., between 6 months and 1, 2, 3, 4, 5 years, or more post-transplant, often after months to years of good graft function. It is caused by a chronic inflammatory and immune response against the graft. For purposes hereof, chronic rejection can include chronic allograft vasculopathy, a term used to refer to fibrosis of the internal blood vessels of the transplanted tissue. As immunosuppressive regimens have reduced the incidence of acute rejection, chronic rejection is becoming more prominent as a cause of graft dysfunction and failure. There is increasing evidence that B-cell production of alloantibody is an important element in the genesis of chronic rejection and graft failure (Kwun J. and Knechtle S J, Transplantation, 88(8):955-61 (2009). Earlier damage to the graft may be a contributing factor leading to chronic processes such as fibrosis that can ultimately lead to chronic rejection.

In some embodiments, a gene therapy described herein, alone or in combination with one or more additional complement inhibitors, is administered to a graft recipient to inhibit graft rejection and/or graft failure.

C. Ischemia/Reperfusion Injury

Ischemia-reperfusion (I/R) injury is an important cause of tissue damage following trauma and in other conditions associated with temporary disruption of blood flow such as myocardial infarction, stroke, severe infection, vascular disease, aneurysm repair, cardiopulmonary bypass, and transplantation.

In the setting of trauma, systemic hypoxemia, hypotension, and local interruption of the blood supply resulting from contusions, compartment syndrome, and vascular injuries cause ischemia that damages metabolically active tissues. Restoration of the blood supply triggers an intense systemic inflammatory reaction that is often more harmful than the ischemia itself. Once the ischemic region is reperfused, factors that are produced and released locally enter the circulatory system and reach remote locations, sometimes causing significant damage to organs not affected by the original ischemic insult, such as the lungs and intestine, leading to single and multiple organ dysfunction. Complement activation occurs soon after reperfusion and is a key mediator of post-ischemic damage, both directly and through its chemoattractive and stimulatory effects on neutrophils. All three major complement pathways are activated and, acting cooperatively or independently, are involved in I/R related adverse events affecting numerous organ systems. In some embodiments of the disclosure, a gene therapy described herein, alone or in combination with one or more additional complement inhibitors, is administered to a subject who has recently (e.g., within the preceding 2, 4, 8, 12, 24, or 48 hours) experienced trauma, e.g., trauma that puts the subject at risk of I/R injury, e.g., due to systemic hypoxemia, hypotension, and/or local interruption of the blood supply. In some embodiments, the subject suffers from spinal cord injury, traumatic brain injury, burn, and/or hemorrhagic shock.

In some embodiments, a gene therapy described herein, alone or in combination with one or more additional complement inhibitors, is administered to a subject prior to, during, or after a surgical procedure. Examples of such procedures include cardiopulmonary bypass, angioplasty, heart valve repair/replacement, aneurysm repair, or other vascular surgeries. A gene therapy described herein, alone or in combination with one or more additional complement inhibitors, may be administered prior to, after, and/or during an overlapping time period with the surgical procedure.

In some embodiments, a gene therapy described herein, alone or in combination with one or more additional complement inhibitors, is administered to a subject who has suffered an MI, thromboembolic stroke, deep vein thrombosis, or pulmonary embolism. A gene therapy described herein, alone or in combination with one or more additional complement inhibitors, may be administered in combination with a thrombolytic agent such as tissue plasminogen activator (tPA) (e.g., alteplase (Activase), reteplase (Retavase), tenecteplase (TNKase)), anistreplase (Eminase), streptokinase (Kabikinase, Streptase), or urokinase (Abbokinase). A gene therapy described herein, alone or in combination with one or more additional complement inhibitors, may be administered prior to, after, and/or during an overlapping time period with the thrombolytic agent.

In some embodiments, a gene therapy described herein, alone or in combination with one or more additional complement inhibitors, is administered to a subject to treat I/R injury.

D. Other Complement-Mediated Disorders

In some embodiments, a gene therapy described herein, alone or in combination with one or more additional complement inhibitors, is introduced into the eye for treatment of an eye disorder such as age-related macular degeneration (AMD), diabetic retinopathy, glaucoma, or uveitis. For example, a gene therapy described herein, alone or in combination with one or more additional complement inhibitors, may be introduced into the vitreous cavity (e.g., by intravitreal injection) or introduced into the subretinal space (e.g., by subretinal injection), for treatment of a subject suffering from or at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with a gene therapy described herein, alone or in combination with one or more additional complement inhibitors, as compared with control (e.g., patients given a sham administration). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD. In some embodiments a gene therapy described herein, alone or in combination with one or more additional complement inhibitors, is administered, e.g., by intravitreal injection or subretinal injection to treat glaucoma, uveitis (e.g., posterior uveitis), or diabetic retinopathy. In some embodiments a gene therapy described herein, alone or in combination with one or more additional complement inhibitors, is introduced into the anterior chamber, e.g., to treat anterior uveitis.

In some embodiments a gene therapy described herein, alone or in combination with one or more additional complement inhibitors described herein, is used to treat a subject suffering from or at risk of an autoimmune disease, e.g., an autoimmune disease mediated at least in part by antibodies against one or more self antigens.

A gene therapy described herein, alone or in combination with one or more additional complement inhibitors described herein may be introduced into the synovial cavity, e.g., in a subject suffering from arthritis (e.g., rheumatoid arthritis).

In some embodiments, a gene therapy described herein, alone or in combination with one or more additional complement inhibitors described herein, is used to treat a subject suffering from or at risk of an intracerebral hemorrhage.

In some embodiments a gene therapy described herein, alone or in combination with one or more additional complement inhibitors described herein, is used to treat a subject suffering from or at risk of myasthenia gravis.

In some embodiments a gene therapy described herein, alone or in combination with one or more additional complement inhibitors described herein, is used to treat a subject suffering from or at risk of neuromyelitis optica (NMO).

In some embodiments a gene therapy described herein, alone or in combination with one or more additional complement inhibitors described herein, is used to treat a subject suffering from or at risk of a disorder affecting the kidney, e.g., the glomeruli of the kidney. In some embodiments the disorder is membranoproliferative glomerulonephritis (MPGN), e.g., MPGN type I, MPGN type II, or MPGN type III. In some embodiments the disorder is IgA nephropathy (IgAN). In some embodiments the disorder is primary membranous nephropathy. In some embodiments the disorder is C3 glomerulopathy. In some embodiments the disorder is characterized by glomerular deposits containing one or more complement activation products, e.g., C3b, in the kidney. In some embodiments treatment as described herein reduces the level of such deposits. In some embodiments a subject suffering from a complement-mediated kidney disorder suffers from proteinuria (an abnormally high level of protein in the urine) and/or an abnormally low glomerular filtration rate (GFR). In some embodiments treatment as described herein results in decreased proteinuria and/or an increased or stabilized GFR.

In some embodiments, a gene therapy described herein, alone or in combination with one or more additional complement inhibitors described herein, is used to treat a subject suffering from or at risk of a neurodegenerative disease. In some embodiments, a gene therapy described herein, alone or in combination with one or more additional complement inhibitors described herein, is used to treat a subject suffering from neuropathic pain or at risk of developing neuropathic pain. In some embodiments, a gene therapy described herein, alone or in combination with one or more additional complement inhibitors described herein, is used to treat a subject suffering from or at risk of rhinosinusitis or nasal polyposis. In some embodiments, a gene therapy described herein, alone or in combination with one or more additional complement inhibitors described herein, is used to treat a subject suffering from or at risk of cancer. In some embodiments, a gene therapy described herein, alone or in combination with one or more additional complement inhibitors described herein, is used to treat a subject suffering from or at risk of sepsis. In some embodiments, a gene therapy described herein, alone or in combination with one or more additional complement inhibitors described herein, is used to treat a subject suffering from or at risk of adult respiratory distress syndrome.

In some embodiments, a gene therapy described herein, alone or in combination with one or more additional complement inhibitors described herein, is used to treat a subject suffering from or at risk of anaphylaxis or infusion reaction. For example, in some embodiments, a subject may be treated prior to, during, or after receiving a drug or a vehicle that may cause anaphylaxis or infusion reaction. In some embodiments, a subject at risk of or suffering from anaphylaxis from a food (e.g., peanut, shellfish, or other food allergens), insect sting (e.g., bee, wasp), is treated with a gene therapy described herein, alone or in combination with one or more additional complement inhibitors described herein.

A gene therapy described herein, alone or in combination with one or more additional complement inhibitors described herein, may be administered locally or systemically, in various embodiments of the disclosure.

In some embodiments, a gene therapy described herein, alone or in combination with one or more additional complement inhibitors described herein, is used to treat a respiratory disease, e.g., asthma or chronic obstructive pulmonary disease (COPD) or idiopathic pulmonary fibrosis. A gene therapy described herein, alone or in combination with one or more additional complement inhibitors described herein, may, for example, be administered to the respiratory tract by inhalation, e.g., as a dry powder or via nebulization, or may be administered by injection, e.g., intravenously, intramuscularly, or subcutaneously, in various embodiments. In some embodiments, a gene therapy described herein, alone or in combination with one or more additional complement inhibitors described herein, is used to treat severe asthma, e.g., asthma that is not sufficiently controlled by bronchodilators and/or inhaled corticosteroids.

In some aspects, methods of treating a complement-mediated disorder, e.g., a chronic complement-mediated disorder, are provided, the methods comprising administering a gene therapy described herein, alone or in combination with one or more additional complement inhibitors described herein, to a subject in need of treatment for the disorder. In some aspects, methods of treating a Th17-associated disorder are provided, the methods comprising administering a gene therapy described herein, alone or in combination with one or more additional complement inhibitors described herein, to a subject in need of treatment for the disorder.

In some aspects, a “chronic disorder” is a disorder that persists for at least 3 months and/or is accepted in the art as being a chronic disorder. In many embodiments, a chronic disorder persists for at least 6 months, e.g., at least 1 year, or more, e.g., indefinitely. One of ordinary skill in the art will appreciate that at least some manifestations of various chronic disorders may be intermittent and/or may wax and wane in severity over time. A chronic disorder may be progressive, e.g., having a tendency to become more severe or affect larger areas over time. A number of chronic complement-mediated disorders are discussed herein. A chronic complement-mediated disorder may be any chronic disorder in which complement activation (e.g., excessive or inappropriate complement activation) is involved, e.g., as a contributing and/or at least partially causative factor. For convenience, disorders are sometimes grouped by reference to an organ or system that is often particularly affected in subjects suffering from the disorder. It will be appreciated that a number of disorders can affect multiple organs or systems, and such classification(s) are in no way limiting. Furthermore, a number of manifestations (e.g., symptoms) may occur in subjects suffering from any of a number of different disorders. Non-limiting information regarding disorders of interest herein may be found, e.g., in standard textbooks of internal medicine such as Cecil Textbook of Medicine (e.g., 23rd edition), Harrison's Principles of Internal Medicine (e.g., 17th edition), and/or standard textbooks focusing on particular areas of medicine, particular body systems or organs, and/or particular disorders.

In some embodiments, a chronic complement-mediated disorder is a Th2-associated disorder. As used herein, a Th2-associated disorder is a disorder characterized by an excessive number and/or excessive or inappropriate activity of CD4+ helper T cells of the Th2 subtype (“Th2 cells”) in the body or a portion thereof, e.g., in at least one tissue, organ, or structure. For example, there may be a predominance of Th2 cells relative to CD4+ helper T cells of the Th1 subtype (“Th1 cells”) e.g., in at least one tissue, organ, or structure affected by a disorder. As known in the art, Th2 cells typically secrete characteristic cytokines such as interleukin-4 (IL-4), interleukin-5 (IL-5), and interleukin-13 (IL-13), while Th1 cells typically secrete interferon-γ (IFN-γ) and tumor necrosis factor $ (TNF $). In some embodiments, a Th2-associated disorder is characterized by excessive production and/or amount of IL-4, IL-5, and/or IL-13, e.g., relative to IFN-γ and/or TNF $ e.g., in at least some at least one tissue, organ, or structure.

In some embodiments, a chronic complement-mediated disorder is a Th17-associated disorder. In some aspects, as described in further detail in PCT/US2012/043845, filed Jun. 22, 2012, entitled “Methods of Treating Chronic Disorders with Complement Inhibitors”, complement activation and Th17 cells participate in a cycle that involves dendritic cells and antibodies and that contributes to maintenance of a pathologic immunologic microenvironment underlying a range of disorders. Without wishing to be bound by any theory, the pathologic immunologic microenvironment, once established, is self-sustaining and contributes to cell and tissue injury.

As used herein, a Th17-associated disorder is a disorder characterized by an excessive number and/or excessive or inappropriate activity of CD4+ helper T cells of the Th17 subtype (“Th17 cells”) in the body or a portion thereof, e.g., in at least one tissue, organ, or structure. For example, there may be a predominance of Th17 cells relative to Th1 and/or Th2 cells, e.g., in at least one tissue, organ, or structure affected by a disorder. In some embodiments a predominance of Th17 cells is a relative predominance, e.g., the ratio of Th17 cells to Th1 cells and/or the ratio of Th17 cells to Th2 cells, is increased relative to normal values. In some embodiments the ratio of Th17 cells to T regulatory cells (CD4+CD25+ regulatory T cells, also termed “Treg cells”), is increased relative to normal values. Formation of Th17 cells and/or activation of Th 17 cells is promoted by various cytokines, e.g., interleukin 6 (IL-6), interleukin 21 (IL-21), interleukin 23 (IL-23), and/or interleukin 1β (IL-1β). Formation of Th17 cells encompasses differentiation of precursor T cells, e.g., naïve CD4+ T cells, towards a Th17 phenotype and their maturation into functional Th17 cells. In some embodiments, formation of Th17 cells encompasses any aspect of development, proliferation (expansion), survival, and/or maturation of Th17 cells. In some embodiments, a Th17-associated disorder is characterized by excessive production and/or amount of IL-6, IL-21, IL-23, and/or IL-1β. Th17 cells typically secrete characteristic cytokines such as interleukin-17A (IL-17A), interleukin-17F (IL-17F), interleukin-21 (IL-21), and interleukin-22 (IL-22). In some embodiments, a Th17-associated disorder is characterized by excessive production and/or amount of a Th17 effector cytokine, e.g., IL-17A, IL-17F, IL-21, and/or IL-22. In some embodiments excessive production or amount of a cytokine is detectable in the blood. In some embodiments excessive production or amount of a cytokine is detectable locally, e.g., in at least one tissue, organ or structure. In some embodiments a Th17-associated disorder is associated with a decreased number of Tregs and/or decreased amount of a Treg-associated cytokine. In some embodiments a Th17 disorder is any chronic inflammatory disease, which term encompasses a range of ailments characterized by self-perpetuating immune insults to a variety of tissues and that seem to be dissociated from the initial insult that caused the ailment (which may be unknown). In some embodiments a Th17-associated disorder is any autoimmune disease. Many if not most “chronic inflammatory diseases” may in fact be auto-immune diseases. Examples of Th17-associated disorders include inflammatory skin diseases such as psoriasis and atopic dermatitis; systemic scleroderma and sclerosis; inflammatory bowel disease (IBD) (such as Crohn's disease and ulcerative colitis); Behcet's Disease; dermatomyositis; polymyositis; multiple sclerosis (MS); dermatitis; meningitis; encephalitis; uveitis; osteoarthritis; lupus nephritis; rheumatoid arthritis (RA), Sjogren's syndrome, multiple sclerosis, vasculitis; central nervous system (CNS) inflammatory disorders, chronic hepatitis; chronic pancreatitis, glomerulonephritis; sarcoidosis; thyroiditis, pathologic immune responses to tissue/organ transplantation (e.g., transplant rejection); COPD, asthma, bronchiolitis, hypersensitivity pneumonitis, idiopathic pulmonary fibrosis (IPF), periodontitis, and gingivitis. In some embodiments a Th17 disease is a classically known auto-immmune disease such as Type I diabetes or psoriasis. In some embodiments a Th17-associated disorder is age-related macular degeneration.

In some embodiments, a chronic complement-mediated disorder is an IgE-associated disorder. As used herein, an “IgE-associated disorder” is a disorder characterized by excessive and/or inappropriate production and/or amount of IgE, excessive or inappropriate activity of IgE producing cells (e.g., IgE producing B cells or plasma cells), and/or excessive and/or inappropriate activity of IgE responsive cells such as eosinophils or mast cells. In some embodiments, an IgE-associated disorder is characterized by elevated levels of total IgE and/or in some embodiments, allergen-specific IgE, in the plasma of a subject and/or locally.

In some embodiments, a chronic complement-mediated disorder is characterized by the presence of autoantibodies and/or immune complexes in the body, which may activate complement via, e.g., the classical pathway. Autoantibodies may, for example, bind to self antigens, e.g., on cells or tissues in the body. In some embodiments, autoantibodies bind to antigens in blood vessels, skin, nerves, muscle, connective tissue, heart, kidney, thyroid, etc. In some embodiments, a subject has neuromyelitis optica and produces an autoantibody (e.g., an IgG autoantibody) to aquaporin 4. In some embodiments, a subject has pemphigoid and produces an autoantibody (e.g., an IgG or IgE autoantibody) to a structural component of the hemidesmosome (e.g., transmembrane collagen XVII (BP180 or BPAG2) and/or plakin family protein BP230 (BPAG1). In some embodiments, a chronic complement-mediated disorder is not characterized by autoantibodies and/or immune complexes.

In some embodiments, a chronic complement-mediated disorder is a respiratory disorder. In some embodiments, a chronic respiratory disorder is asthma or chronic obstructive pulmonary disease (COPD). In some embodiments, a chronic respiratory disorder is pulmonary fibrosis (e.g., idiopathic pulmonary fibrosis), radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, or bronchiolitis obliterans. In some embodiments, the disclosure provides a method of treating a subject in need of treatment for a chronic respiratory disorder, e.g., asthma, COPD, pulmonary fibrosis, radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, or bronchiolitis obliterans, the method comprising administering a gene therapy described herein, alone or in combination with one or more additional complement inhibitors described herein, to a subject in need of treatment for the disorder.

In some embodiments, a chronic complement-mediated disorder is allergic rhinitis, rhinosinusitis, or nasal polyposis. In some embodiments, the disclosure provides a method of treating a subject in need of treatment for allergic rhinitis, rhinosinusitis, or nasal polyposis, the method comprising administering a gene therapy described herein, alone or in combination with one or more additional complement inhibitors described herein, to a subject in need of treatment for the disorder.

In some embodiments, a chronic complement-mediated disorder is a disorder that affects the musculoskeletal system. Examples of such disorders include inflammatory joint conditions (e.g., arthritis such as rheumatoid arthritis or psoriatic arthritis, juvenile chronic arthritis, spondyloarthropathies Reiter's syndrome, gout). In some embodiments, a musculoskeletal system disorder results in symptoms such as pain, stiffness and/or limitation of motion of the affected body part(s). Inflammatory myopathies include dermatomyositis, polymyositis, and various others are disorders of chronic muscle inflammation of unknown etiology that result in muscle weakness. In some embodiments, a chronic complement-mediated disorder is myasthenia gravis. In some embodiments, the disclosure provides a method of treating any of the foregoing disorders affecting the musculoskeletal system, the method comprising administering a gene therapy described herein, alone or in combination with one or more additional complement inhibitors described herein, to a subject in need of treatment for the disorder.

In some embodiments, a chronic complement-mediated disorder is a disorder that affects the integumentary system. Examples of such disorders include, e.g., atopic dermatitis, psoriasis, pemphigoid, pemphigus, systemic lupus erythematosus, dermatomyositis, scleroderma, sclerodermatomyositis, Sjagren syndrome, and chronic urticaria. In some aspects, the disclosure provides a method of treating any of the foregoing disorders affecting the integumentary system, the method comprising administering a gene therapy described herein, alone or in combination with one or more additional complement inhibitors described herein, to a subject in need of treatment for the disorder.

In some embodiments, a chronic complement-mediated disorder affects the nervous system, e.g., the central nervous system (CNS) and/or peripheral nervous system (PNS). Examples of such disorders include, e.g., multiple sclerosis, other chronic demyelinating diseases (e.g., neuromyelits optica), amyotrophic lateral sclerosis, chronic pain, stroke, allergic neuritis, Huntington's disease, Alzheimer's disease, and Parkinson's disease. In some embodiments, the disclosure provides a method of treating any of the foregoing disorders affecting the nervous system, the method comprising administering a gene therapy described herein, alone or in combination with one or more additional complement inhibitors described herein, to a subject in need of treatment for the disorder.

In some embodiments, a chronic complement-mediated disorder affects the circulatory system. For example, in some embodiments the disorder is a vasculitis or other disorder associated with vessel inflammation, e.g., blood vessel and/or lymph vessel inflammation. In some embodiments, a vasculitis is polyarteritis nodosa, Wegener's granulomatosis, giant cell arteritis, Churg-Strauss syndrome, microscopic polyangiitis, Henoch-Schonlein purpura, Takayasu's arteritis, Kawasaki disease, or Behcet's disease. In some embodiments, a subject, e.g., a subject in need of treatment for vasculitis, is positive for antineutrophil cytoplasmic antibody (ANCA).

In some embodiments, a chronic complement-mediated disorder affects the gastrointestinal system. For example, the disorder may be inflammatory bowel disease, e.g., Crohn's disease or ulcerative colitis. In some embodiments, the disclosure provides a method of treating a chronic complement-mediated disorder that affects the gastrointestinal system, the method comprising administering a gene therapy described herein, alone or in combination with one or more additional complement inhibitors described herein, to a subject in need of treatment for the disorder.

In some embodiments, a chronic complement-mediated disorder is a thyroiditis (e.g., Hashimoto's thyroiditis, Graves' disease, post-partum thyroiditis), myocarditis, hepatitis (e.g., hepatitis C), pancreatitis, glomerulonephritis (e.g., membranoproliferative glomerulonephritis or membranous glomerulonephritis), or panniculitis.

In some embodiments, the disclosure provides methods of treating a subject suffering from chronic pain, the methods comprising administering a gene therapy described herein, alone or in combination with one or more additional complement inhibitors described herein, to a subject in need thereof. In some embodiments, a subject suffers from neuropathic pain. Neuropathic pain has been defined as pain initiated or caused by a primary lesion or dysfunction in the nervous system, in particular, pain arising as a direct consequence of a lesion or disease affecting the somatosensory system. For example, neuropathic pain may arise from lesions that involve the somatosensory pathways with damage to small fibres in peripheral nerves and/or to the spino-thalamocortical system in the CNS. In some embodiments, neuropathic pain arises from autoimmune disease (e.g., multiple sclerosis), metabolic disease (e.g., diabetes), infection (e.g., viral disease such as shingles or HIV), vascular disease (e.g., stroke), trauma (e.g., injury, surgery), or cancer. For example, neuropathic pain can be pain that persists after healing of an injury or after cessation of a stimulus of peripheral nerve endings or pain that arises due to damage to nerves. Exemplary conditions of or associated with neuropathic pain include painful diabetic neuropathy, post-herpetic neuralgia (e.g., pain persisting or recurring at the site of acute herpes zoster 3 or more months after the acute episode), trigeminal neuralgia, cancer related neuropathic pain, chemotherapy-associated neuropathic pain, HIV-related neuropathic pain (e.g., from HIV neuropathy), central/post-stroke neuropathic pain, neuropathy associated with back pain, e.g., low back pain (e.g., from radiculopathy such as spinal root compression, e.g., lumbar root compression, which compression may arise due to disc herniation), spinal stenosis, peripheral nerve injury pain, phantom limb pain, polyneuropathy, spinal cord injury related pain, myelopathy, and multiple sclerosis. In certain embodiments of the disclosure, a gene therapy described herein, alone or in combination with one or more additional complement inhibitors described herein, is administered according to a dosing schedule to treat neuropathic pain in a subject with one or more of the afore-mentioned conditions.

In some embodiments, a chronic complement-mediated disorder is a chronic eye disorder. In some embodiments, the chronic eye disorder is characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. Macular degeneration related conditions include, e.g., age-related macular degeneration (AMD). In some embodiments, a subject is in need of treatment for wet AMD. In some embodiments, a subject is in need of treatment for dry AMD. In some embodiments, a subject is in need of treatment for geographic atrophy (GA). In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. In some embodiments, the chronic eye disorder is an eye disorder characterized by optic nerve damage (e.g., optic nerve degeneration), such as glaucoma.

As noted above, in some embodiments, the chronic respiratory disease is asthma. Information regarding risk factors, epidemiology, pathogenesis, diagnosis, current management of asthma, etc., may be found, e.g., in “Expert Panel Report 3: Guidelines for the Diagnosis and Management of Asthma”. National Heart Lung and Blood Institute. 2007. http://www.nhlbi.nih.gov/guidelines/asthma/asthgdln.pdf. (“NHLBI Guidelines”; www.nhlbi.nih.gov/guidelines/asthma/asthgdln.htm), Global Initiative for Asthma, Global Strategy for Asthma Management and Prevention 2010 “GINA Report”) and/or standard textbooks of internal medicine such as Cecil Textbook of Medicine (20th edition), Harrison's Principles of Internal Medicine (17th edition), and/or standard textbooks focusing on pulmonary medicine. Asthma is a chronic inflammatory disorder of the airways in which many cells and cellular elements play a role, such as, mast cells, eosinophils, T lymphocytes, macrophages, neutrophils, and epithelial cells Asthmatic individuals experience recurrent episodes associated with symptoms such as wheezing, breathlessness (also termed dyspnea or shortness of breath), chest tightness, and coughing. These episodes are usually associated with widespread but variable airflow obstruction that is often reversible, either spontaneously or with treatment. The inflammation also causes an associated increase in the existing bronchial hyperresponsiveness to a variety of stimuli. Airway hyperresponsiveness (an exaggerated bronchoconstrictor response to stimuli) is a typical feature of asthma. In general, airflow limitation results from bronchoconstriction and airway edema. Reversibility of airflow limitation may be incomplete in some patients with asthma. For example, airway remodeling can lead to fixed airway narrowing. Structural changes can include thickening of the sub-basement membrane, subepithelial fibrosis, airway smooth muscle hypertrophy and hyperplasia, blood vessel proliferation and dilation, and mucous gland hyperplasia, and hypersecretion.

Individuals with asthma may experience exacerbations, which are identified as events characterized by a change from the individual's previous status. Severe asthma exacerbations can be defined as events that require urgent action on the part of the individual and his/her physician to prevent a serious outcome, such as hospitalization or death from asthma. For example, a severe asthma exacerbation may require use of systemic corticosteroids (e.g., oral corticosteroids) in a subject whose asthma is usually well controlled without OCS or may require an increase in a stable maintenance dose. Moderate asthma exacerbations can be defined as events that are troublesome to the subject, and that prompt a need for a change in treatment, but that are not severe. These events are clinically identified by being outside the subject's usual range of day-to-day asthma variation.

Current medications for asthma are typically categorized into two general classes: long-term control medications (“controller medications”) such as inhaled corticosteroids (ICS), oral corticosteroids (OCS), long-acting bronchodilators (LABAs), leukotriene modifiers (e.g., leukotriene receptor antagonists or leukotriene synthesis inhibitors, anti-IgE antibodies (omalizumab (Xolair®)), cromolyn and nedocromil, which are used to achieve and maintain control of persistent asthma and quick-relief medications such as short-acting bronchodilators (SABAs), which are used to treat acute symptoms and exacerbations. For purposes of the present invention, these treatments may be referred to as “conventional therapy”. Treatment of exacerbations may also include increasing the dose and/or intensity of controller medication therapy. For example, a course of OCS can be used to regain asthma control. Current guidelines mandate daily administration of controller medication or, in many cases, administration of multiple doses of controller medication each day for subjects with persistent asthma (with the exception of Xolair, which is administered every 2 or 4 weeks).

A subject is generally considered to have persistent asthma if the subject suffers from symptoms on average more than twice a week and/or typically uses a quick relief medication (e.g., SABA) more than twice a week for symptom control. “Asthma severity” can be classified based on the intensity of treatment required to control the subject's asthma once relevant comorbidities have been treated and inhaler technique and adherence have been optimized (see, e.g., GINA Report; Taylor, DR, Eur Respir J 2008; 32:545-554). The description of treatment intensity can be based on the medications and doses recommended in the stepwise treatment algorithm found in guidelines such as NHLBI Guidelines 2007, GINA Report, and their predecessors and/or in standard medical textbooks. For example, asthma can be classified as intermittent, mild, moderate, or severe as indicated in Table 8, where “treatment” refers to treatment sufficient to achieve subject's best level of asthma control. (It will be understood that the categories of mild, moderate, and severe asthma in general imply persistent rather than intermittent asthma). One of ordinary skill in the art will appreciate that Table 8 is exemplary, and that not all of these medications will be available in all healthcare systems, which may affect the assessment of asthma severity in some environments. It will also be appreciated that other emerging or new approaches may affect the classification of mild/moderate asthma. However, the same principle, of mild asthma being defined by the ability to achieve good control using very low-intensity treatment and severe asthma being defined by the requirement for high-intensity treatment, can still be applied. Asthma severity can also or alternately be classified based on intrinsic intensity of the disease in the absence of treatment (see, e.g., NHBLI Guidelines 2007). Assessment can be made on the basis of current spirometry and the patient's recall of symptoms over the previous 2-4 weeks. Parameters of current impairment and future risk may be assessed and included in a determination of the level of asthma severity. In some embodiments, asthma severity is defined as shown in FIG. 3.4(a), 3.4(b), 3.4(c) of the NHBLI Guidelines, for individuals 0-4, 5-11, or ≥12 years of age, respectively.

TABLE 8
Treatment-based Asthma Classification

| Asthma | |
|---|---|
| Classification | Treatment |
| Intermittent | SABA as needed (typically no more than twice a week) |
| Mild | Low-dose ICS or other low-intensity treatment (e.g., |
| | LTRA, cromolyn, nedocromil, theophylline) |
| Moderate | Low to moderate dose ICS and LABA or other extra |
| | treatment |
| Severe | High-intensity treatment (high-dose ICS and LABA ± |
| | oral corticosteroids and/or other extra treatment) |

“Asthma control” refers to the extent to which the manifestations of asthma have been reduced or removed by treatment (whether pharmacological or non-pharmacological). Asthma control can be assessed based on factors such as symptom frequency, nighttime symptoms, objective measures of lung function such as spirometry parameters (e.g., % FEV₁ of predicted, FEV₁ variability, requirement for use of SABA for symptom control. Parameters of current impairment and future risk may be assessed and included in a determination of the level of asthma control. In some embodiments, asthma control is defined as shown in FIG. 4.3(a), 4.3(b), or 4.3(c) of NHBLI Guidelines, for individuals 0-4, 5-11, or ≥12 years of age, respectively.

In general, one of ordinary skill in the art can select an appropriate means of determining asthma severity level and/or degree of control, and any classification scheme considered reasonable by those of ordinary skill in the art can be used.

In some embodiments of the disclosure, a subject suffering from persistent asthma is treated with a gene therapy described herein, alone or in combination with one or more additional complement inhibitors described herein, using a dosing regimen. In some embodiments, the subject suffers from mild or moderate asthma. In some embodiments, the subject suffers from severe asthma. In some embodiments, a subject has asthma that is not well controlled using conventional therapy. In some embodiments, a subject has asthma that, when treated using conventional therapy, requires use of ICS in order to be well controlled. In some embodiments, a subject has asthma that fails to be well controlled despite use of ICS. In some embodiments, a subject has asthma that, if treated using conventional therapy, would require use of OCS in order to be well controlled. In some embodiments, a subject has asthma that fails to be well controlled despite use of high intensity conventional therapy that includes OCS. In some embodiments, a gene therapy described herein, alone or in combination with one or more additional complement inhibitors described herein, is administered as a controller medication or allow the subject to avoid using or reduce their dose of a conventional controller medication.

In some embodiments, the subject suffers from allergic asthma, which is the case for most asthmatic individuals. In some embodiments, an asthmatic subject is considered to have allergic asthma if a non-allergic trigger for the asthma (e.g., cold, exercise) is not known and/or is not identified in a standard diagnostic evaluation. In some embodiments, an asthmatic subject is considered to have allergic asthma if the subject (i) reproducibly develops asthma symptoms (or worsening of asthma symptoms) following exposure to an allergen or allergen(s) to which the subject is sensitive; (ii) exhibits IgE specific for an allergen or allergen(s) to which the subject is sensitive; (iii) exhibits a positive skin-prick test to an allergen or allergen(s) to which the subject is sensitive; and/or (iv) exhibits other symptom(s) of characteristic(s) consistent with atopy such as allergic rhinitis, eczema, or elevated total serum IgE. It will be appreciated that a specific allergic trigger may not be identified but may be suspected or inferred if the subject experiences worsening symptoms in particular environments, for example.

Allergen challenge by inhalation is a technique that is widely used in evaluating allergic airway disease. Inhalation of allergen leads to cross-linking of allergen-specific IgE bound to IgE receptors on, e.g., mast cells and basophils. Activation of secretory pathways ensues, resulting in release of mediators of bronchoconstriction and vascular permeability. Individuals with allergic asthma may develop various manifestations following allergen challenge, e.g., early asthmatic response (EAR), late asthmatic response (LAR), airway hyperreactivity (AHR), and airway eosinophilia, each of which can be detected and quantified as known in the art. For example, airway eosiphophilia may be detected as an increase in eosinophils in sputum and/or BAL fluid. The EAR, sometimes referred to as the immediate asthmatic response (JAR), is a response to allergen challenge by inhalation that becomes detectable shortly after the inhalation, typically within 10 minutes (min) of the inhalation, e.g., as a decrease in FEV₁. The EAR typically reaches a maximum within 30 min and resolves within 2-3 hours (h) post-challenge. For example, a subject may be considered to exhibit a “positive” EAR if his/her FEV₁ decreases by at least 15%, e.g., at least 20%, within this time window relative to baseline FEV₁ (where “baseline” in this context refers to conditions before the challenge, e.g., conditions equivalent to the subject's usual condition when not experiencing an asthma exacerbation and not exposed to allergic stimuli to which the subject is sensitive). The late asthmatic response (LAR) typically starts between 3 h and 8 h post-challenge and is characterized by cellular inflammation of the airway, increased bronchiovascular permeability, and mucus secretion. It is typically detected as a decrease in FEV₁, which may be greater in magnitude than that associated with the EAR and potentially more clinically important. For example, a subject may be considered to exhibit a “positive” LAR if his/her FEV₁ decreases by at least 15%, e.g., at least 20%, relative to baseline FEV₁ within the relevant time period as compared with baseline FEV₁. A delayed airway response (DAR) may occur beginning between about 26 and 32 h, reaching a maximum between about 32 and 48 h and resolving within about 56 h after the challenge (Pelikan, Z. Ann Allergy Asthma Immunol. 2010, 104(5):394-404).

In some embodiments, the chronic respiratory disorder is chronic obstructive pulmonary disease (COPD). COPD encompasses a spectrum of conditions characterized by airflow limitation that is not fully reversible even with therapy and is usually progressive. Symptoms of COPD include dyspnea (breathlessness), decreased exercise tolerance, cough, sputum production, wheezing, and chest tightness. Persons with COPD can experience episodes of acute (e.g., developing over course of less than a week and often over the course of 24 hours or less) worsening of symptoms (termed COPD exacerbations) that can vary in frequency and duration and are associated with significant morbidity. They may be triggered by events such as respiratory infection, exposure to noxious particles, or may have an unknown etiology. Smoking is the most commonly encountered risk factor for COPD, and other inhalational exposures can also contribute to development and progression of the disease. The role of genetic factors in COPD is an area of active research. A small percentage of COPD patients have a hereditary deficiency of alpha-I antitrypsin, a major circulating inhibitor of serine proteases, and this deficiency can lead to a rapidly progressive form of the disease.

Characteristic pathophysiologic features of COPD include narrowing of and structural changes in the small airways and destruction of lung parenchyma (in particular around alveoli), most commonly due to chronic inflammation. The chronic airflow limitation observed in COPD typically involves a mixture of these factors, and their relative importance in contributing to airflow limitation and symptoms varies from person to person. The term “emphysema” refers to enlargement of the air spaces (alveoli) distal to the terminal bronchioles, with destruction of their walls. It should be noted that the term “emphysema” is often used clinically to refer to the medical condition associated with such pathological changes. Some individuals with COPD have chronic bronchitis, which is defined in clinical terms as a cough with sputum production on most days for 3 months of a year, for 2 consecutive years. Further information regarding risk factors, epidemiology, pathogenesis, diagnosis, and current management of COPD may be found, e.g., in “Global Strategy for the Diagnosis, Management, and Prevention of Chronic Obstructive Pulmonary Disease” (updated 2009) available on the Global Initiative on Chronic Obstructive Pulmonary Disease, Inc. (GOLD) website (www.goldcopd.org), also referred to herein as the “GOLD Report”, the American Thoracic Society/European Respiratory Society Guidelines (2004) available on the ATS website at www.thoracic.org/clinical/copd-guidelines/resources/copddoc.pdf, referred to herein as “ATC/ERS COPD Guidelines” and standard textbooks of internal medicine such as Cecil Textbook of Medicine (20^{th} edition), Harrison's Principles of Internal Medicine (17′ edition), and/or standard textbooks focusing on pulmonary medicine.

In some embodiments methods disclosed herein inhibit (interfere with, disrupt) the DC-Th17-B-Ab-C-DC cycle discussed above. For example, administration of a gene therapy described herein, alone or in combination with one or more additional complement inhibitors described herein, may break the cycle by which complement stimulates DC cells to promote the Th17 phenotype. As a result, the number and/or activity of Th17 cells diminishes, which in turn reduces the amount of Th17-mediated stimulation of B cells and polyclonal antibody production. In some embodiments, these effects result in “resetting” the immunological microenvironment to a more normal, less pathological state. As described in Example 1 of PCT/US2012/043845 (WO/2012/178083) and US Publ. No. 20140371133 evidence supporting the capacity of complement inhibition to have a prolonged inhibitory effect on Th17-associated cytokine production has been obtained in an animal model of asthma.

In some embodiments, inhibiting the DC-Th17-B-Ab-C-DC cycle has a disease-modifying effect. Without wishing to be bound by any theory, rather than merely treating symptoms of a disorder, inhibiting the DC-Th17-B-Ab-C-DC cycle may interfere with fundamental pathologic mechanisms that may contribute to ongoing tissue damage even when symptoms are well controlled and/or that may contribute to exacerbations of the disease. In some embodiments, inhibiting the DC-Th17-B-Ab-C-DC cycle causes a chronic disorder to go into remission. In some embodiments, remission refers to a state of absence or substantial absence of disease activity in a subject with a chronic disorder, with the possibility of return of disease. In some embodiments remission may be sustained for a prolonged period of time (e.g., at least 6 months, e.g., 6-12 months, 12-24 months, or more) in the absence of continued therapy or with a reduced dose or increased dosing interval. In some aspects, inhibition of complement may change the immunological micro-environment of a tissue that is rich in Th17 cells and modify it into a micro-environment that is rich in regulatory T cells (Tregs). Doing so could allow the immune system to “reset” itself and go into a state of remission. In some embodiments, for example, remission may be sustained until occurrence of a triggering event. A triggering event may be, for example, an infection (which may result in production of polyclonal antibodies that react both with an infectious agent and a self protein), exposure to particular environmental conditions (e.g., high levels of air pollutants such as ozone or particulate matter or components of smoke such as cigarette smoke, allergens), etc. Genetic factors may play a role. For example, individuals having particular alleles of genes encoding complement components may have a higher baseline level of complement activity, a more reactive complement system and/or a lower baseline level of endogenous complement regulatory protein activity. In some embodiments an individual has a genotype associated with increased risk of AMD. For example, the subject may have a polymorphism in a gene encoding a complement protein or complement regulatory protein, e.g., CFH, C3, factor B, wherein the polymorphism is associated with an increased risk of AMD.

In some embodiments an immunologic microenvironment may become progressively more polarized towards a pathological state over time, e.g., in a subject who has not yet developed symptoms of a chronic disorder or in a subject who has developed the disorder and has been treated as described herein. Such a transition may occur stochastically (e.g., due at least in part to apparently random fluctuations in antibody levels and/or affinity) and/or as a result of accumulated “sub-threshold” trigger events that are not of sufficient intensity to trigger a symptomatic outbreak of a disorder.

In some embodiments it is contemplated that a relatively short course of a gene therapy described herein, alone or in combination with one or more additional complement inhibitors described herein, e.g., between 1 week and 6 weeks, e.g., about 2-4 week, may provide a long-lasting benefit. In some embodiments, a remission is achieved for a prolonged period of time, e.g., 1-3 months, 3-6 months, 6-12 months, 12-24 months, or more. In some embodiments, a gene therapy described herein is administered to a subject only once or twice and achieves a benefit lasting at least 1 month, 2 months, 3 months, 6 months, 9 months, 12 months, or longer. In some embodiments a subject may be monitored and/or treated prophylactically before recurrence of symptoms. For example, a subject may be treated prior to or upon exposure to a triggering event. In some embodiments a subject may be monitored, e.g., for an increase in a biomarker, e.g., a biomarker comprising an indicator of Th17 cells or Th17 cell activity, or complement activation, and may be treated upon increase in the level of such biomarker. See, e.g., PCT/US2012/043845 for further discussion.

### Combination Therapy

In some aspects, methods of the present disclosure involve administering a gene therapy described herein, alone or in combination with one or more additional complement inhibitors. In some embodiments, a gene therapy is administered to a subject already receiving therapy with another complement inhibitor; in some embodiments, another complement inhibitor is administered to a subject receiving a gene therapy. In some embodiments, both a gene therapy and another complement inhibitor are administered to the subject.

In some embodiments administration of a gene therapy may allow for administering a reduced dosing regimen of (e.g., involving a smaller amount in an individual dose, reduced frequency of dosing, reduced number of doses, and/or reduced overall exposure to) a second complement inhibitor, as compared to administration of a second complement inhibitor as single therapy. Without wishing to be bound by any theory, in some embodiments a reduced dosing regimen of a second complement inhibitor may avoid one or more undesired adverse effects that could otherwise result.

In some aspects, administration of a gene therapy in combination with a second complement inhibitor can reduce the amount of C3 in the subject's blood sufficiently such that a reduced dosing regimen of a gene therapy and/or the second complement inhibitor is required to achieve a desired degree of complement inhibition.

In some embodiments such a reduced dose can be administered in a smaller volume, or using a lower concentration, or using a longer dosing interval, or any combination of the foregoing, as compared to administration of a gene therapy or a second complement inhibitor as single therapy.

Any complement inhibitor, e.g., a complement inhibitor known in the art, can be administered in combination with a gene therapy described herein. In some embodiments, a complement inhibitor is compstatin or a compstatin analog.

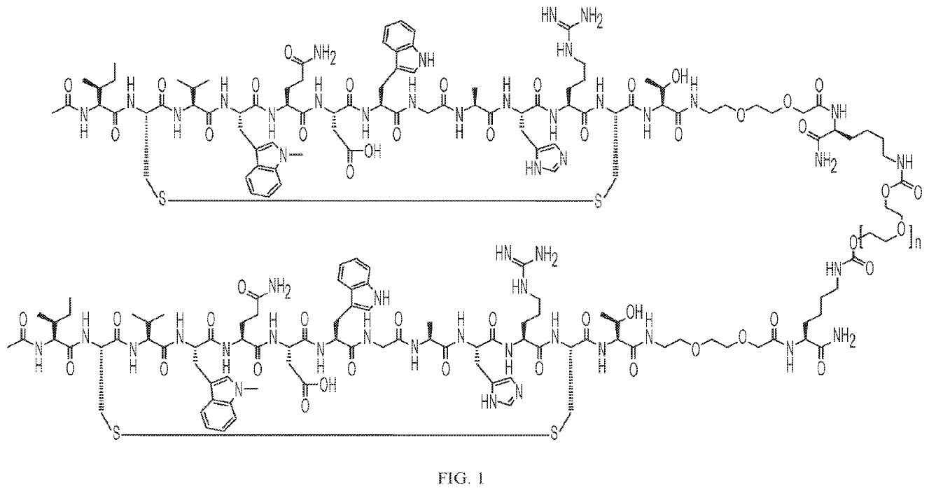

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus. The term “compstatin” is used herein consistently with such usage. Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345). Additional compstatin analogs are described in, e.g., WO 2012/155107, WO 2014/078731, and WO 2019/166411. In certain embodiments, a compstatin analog is pegcetacoplan (“APL-2”), having the structure of the compound of FIG. 1 with n of about 800 to about 1100 and a PEG having an average molecular weight of about 40 kD. Pegcetacoplan is also referred to as Poly(oxy-1,2-ethanediyl), α-hydro-(ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2-->12)-(disulfide); or 0,0′-bis[(S²,S¹²-cyclo{N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6.15}-carbonyl]polyethylene glycol (n=800-1100).

In some embodiments, a complement inhibitor is an antibody, e.g., an anti-C3 and/or anti-C5 antibody, or a fragment thereof. In some embodiments, an antibody fragment may be used to inhibit C3 or C5 activation. The fragmented anti-C3 or anti-C5 antibody may be Fab′, Fab′(2), Fv, or single chain Fv. In some embodiments, the anti-C3 or anti-C5 antibody is monoclonal. In some embodiments, the anti-C3 or anti-C5 antibody is polyclonal. In some embodiments, the anti-C3 or anti-C5 antibody is de-immunized. In some embodiments the anti-C3 or anti-C5 antibody is a fully human monoclonal antibody. In some embodiments, the anti-C5 antibody is eculizumab. In some embodiments, a complement inhibitor is an antibody, e.g., an anti-C3 and/or anti-C5 antibody, or a fragment thereof.

In some embodiments, a complement inhibitor is a polypeptide inhibitor and/or a nucleic acid aptamer (see, e.g., U.S. Publ. No. 20030191084). Exemplary polypeptide inhibitors include an enzyme that degrades C3 or C3b (see, e.g., U.S. Pat. No. 6,676,943). Additional polypeptide inhibitors include mini-factor H (see, e.g., U.S. Publ. No. 20150110766), Efb protein or complement inhibitor (SCIN) protein from Staphylococcus aureus, or a variant or derivative or mimetic thereof (see, e.g., U.S. Publ. 20140371133).

A variety of other complement inhibitors can also be used in various embodiments of the disclosure. In some embodiments, the complement inhibitor is a naturally occurring mammalian complement regulatory protein or a fragment or derivative thereof. For example, the complement regulatory protein may be CR1, DAF, MCP, CFH, or CFI. In some embodiments, the complement regulatory polypeptide is one that is normally membrane-bound in its naturally occurring state. In some embodiments, a fragment of such polypeptide that lacks some or all of a transmembrane and/or intracellular domain is used. Soluble forms of complement receptor 1 (sCR1), for example, can also be used. For example the compounds known as TP10 or TP20 (Avant Therapeutics) can be used. C1 inhibitor (C1-INH) can also be used. In some embodiments a soluble complement control protein, e.g., CFH, is used.

Inhibitors of C1s can also be used. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 73) or KTISKAKGQPREPQVYT (SEQ ID NO: 74) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method of treating a subject, comprising administering to a cell of a subject:
(i) a base editor comprising a fusion protein comprising an endonuclease (e.g., a Cas endonuclease) and a deaminase; and
(ii) a gRNA (e.g., a single guide RNA (sgRNA)) comprising a targeting domain comprising a nucleotide sequence that is complementary to a portion of a human C3 gene,
wherein after the administering step, the cell and/or the subject exhibits reduced expression and/or activity of C3 protein (e.g., reduced by about 10%, 20%, 30%, 40%, 50%, 60%, 70%, 80%, or 90%), relative to a control.

2. The method of claim 1, wherein the portion of the human C3 gene comprises a nucleotide sequence within an exon of SEQ ID NO:1.

3. The method of claim 1, wherein the portion of the human C3 gene comprises a nucleotide sequence within an intron of SEQ ID NO:1.

4. The method of any one of claims 1-3, wherein the gRNA targets the base editor to one or more base positions recited in Table 2, 3 or 4.

5. The method of any one of claims 1-4, wherein after the administering step, the human C3 gene comprises a base edit, relative to a wildtype human C3 gene, from a C to a T; from a G to an A; from a T to a C; or from an A to a G at one or more base positions recited in Table 2, 3 or 4.

6. The method of any one of claims 1-5, wherein after the administering step, the human C3 gene comprises a genomic edit, relative to a wildtype human C3 gene, of a nonstop codon to a stop codon at one or more base positions recited in Table 2, 3, or 4.

7. The method of any one of claims 1-6, wherein the reduced activity of the C3 protein comprises reduced thioester domain activity.

8. The method of any one of claims 1-7, wherein after the administering step, the cell or the subject expresses a mutant C3 protein, and a level or rate of cleavage of the mutant C3 protein by a C3 convertase is reduced (e.g., reduced by about 10%, 20%, 30%, 40%, 50%, 60%, 70%, 80%, or 90%), relative to level or rate of cleavage of a wildtype C3 protein by the C3 convertase.

9. The method of any one of claims 1-8, wherein the Cas endonuclease is a nuclease inactive Cas endonuclease.

10. The method of any one of claims 1-8, wherein the Cas endonuclease is a nickase.

11. The method of claim 10, wherein the nickase is a Cas9 nickase.

12. The method of any one of claims 1-11, wherein the deaminase is a deaminase from the apolipoprotein B mRNA-editing complex (APOBEC) family deaminase.

13. The method of claim 12, wherein the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase.

14. The method of any one of claims 1-13, comprising administering a nucleotide sequence encoding the base editor.

15. The method of claim 14, comprising administering a viral vector comprising the nucleotide sequence encoding the base editor.

16. The method of any one of claims 1-15, comprising administering a viral vector comprising the gRNA.

17. The method of claim 15 or 16, comprising administering a viral vector comprising the nucleotide sequence encoding the base editor and comprising the gRNA.

18. The method of any one of claims 1-13, comprising administering a ribonucleoprotein (RNP) complex comprising the base editor and the gRNA.

19. The method of any one of claims 1-18, wherein the subject has or suffers from a complement-mediated disorder.

20. A method of editing a human C3 gene in a cell, comprising contacting a cell with, or administering to a subject:
(i) a base editor comprising a fusion protein comprising an endonuclease (e.g., a Cas endonuclease) and a deaminase; and
(ii) a gRNA (e.g., a single guide RNA (sgRNA)) comprising a targeting domain comprising a nucleotide sequence that is complementary to a portion of the human C3 gene,
wherein after the contacting or administering step, the cell comprises a human C3 gene comprising at least one genomic edit.

21. The method of claim 20, wherein after the administering step, the cell and/or the subject exhibits reduced expression and/or activity of C3 protein (e.g., reduced by about 10%, 20%, 30%, 40%, 50%, 60%, 70%, 80%, or 90%), relative to a control.

22. The method of claim 20 or 21, wherein the portion of the human C3 gene comprises a nucleotide sequence within an exon of SEQ ID NO:1.

23. The method of claim 20 or 21, wherein the portion of the human C3 gene comprises a nucleotide sequence within an intron of SEQ ID NO:1.

24. The method of any one of claims 20-23, wherein the gRNA targets the base editor to one or more base positions recited in Table 2, 3 or 4.

25. The method of any one of claims 20-24, wherein after the administering step, the human C3 gene comprises a base edit, relative to a wildtype human C3 gene, from a C to a T; from a G to an A; from a T to a C; or from an A to a G at one or more base positions recited in Table 2, 3 or 4.

26. The method of any one of claims 20-25, wherein after the administering step, the human C3 gene comprises a genomic edit, relative to a wildtype human C3 gene, of a nonstop codon to a stop codon at one or more base positions recited in Table 2, 3, or 4.

27. The method of any one of claims 20-26, wherein the reduced activity of the C3 protein comprises reduced thioester domain activity.

28. The method of any one of claims 20-27, wherein after the administering step, the cell or the subject expresses a mutant C3 protein, and a level or rate of cleavage of the mutant C3 protein by a C3 convertase is reduced (e.g., reduced by about 10%, 20%, 30%, 40%, 50%, 60%, 70%, 80%, or 90%), relative to level or rate of cleavage of a wildtype C3 protein by the C3 convertase.

29. The method of any one of claims 20-28, wherein the Cas endonuclease is a nuclease inactive Cas endonuclease.

30. The method of any one of claims 20-28, wherein the Cas endonuclease is a nickase.

31. The method of claim 30, wherein the nickase is a Cas9 nickase.

32. The method of any one of claims 20-31, wherein the deaminase is a deaminase from the apolipoprotein B mRNA-editing complex (APOBEC) family deaminase.

33. The method of claim 32, wherein the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase.

34. The method of any one of claims 20-33, comprising administering a nucleotide sequence encoding the base editor.

35. The method of claim 34, comprising administering a viral vector comprising the nucleotide sequence encoding the base editor.

36. The method of any one of claims 20-35, comprising administering a viral vector comprising the gRNA.

37. The method of claim 35 or 36, comprising administering a viral vector comprising the nucleotide sequence encoding the base editor and comprising the gRNA.

38. The method of any one of claims 20-33, comprising administering a ribonucleoprotein (RNP) complex comprising the base editor and the gRNA.

39. The method of any one of claims 20-38, wherein the subject has or suffers from a complement-mediated disorder.

40. A composition comprising:
(i) a base editor comprising a fusion protein comprising an endonuclease (e.g., a Cas endonuclease) and a deaminase; and
(ii) a gRNA (e.g., a single guide RNA (sgRNA)) comprising a targeting domain comprising a nucleotide sequence that is complementary to a portion of the human C3 gene.

41. The composition of claim 40, wherein the gRNA targets the base editor to one or more base positions recited in Table 2, 3 or 4.

42. A cell comprising the composition of claim 40 or 41.

43. A progeny cell of the cell of claim 42.

44. A method of reducing complement activation in a subject (e.g., reducing by about 10%, 20%, 30%, 40%, 50%, 60%, 70%, 80%, or 90%), relative to a control, the method comprising administering to the subject the composition of claim 40 or 41.

45. The method of any one of claims 1-39, wherein the cell is a hepatic cell.

46. The cell of claim 42 or 43, wherein the cell is a hepatic cell.
